(19) United States
(12) Patent Application Publication
(10) Pub. No.: US 2009/0023721 A1
(43) Pub. Date: Jan. 22, 2009

(54) Novel Substituted Tetracyclic Tetrahydrofuran, Pyrrolidine and Tetrahydrothiophene Derivatives and Their Use as a Medicament

(76) Inventors: Antonius Adrianus Hendrikus Petrus Megens, Beerse (BE); Andres Avelino Trabanco-Suarez, Olias del Rey (Toledo) (ES); Jose Maria Cid-Nunez, Toledo (ES); Hua Mao, Fayetteville, AR (US); Sushil Chandra Jha, Pune (IN); Francisco Javier Fernandez-Gadea, Toledo (ES); Mohamed Koukni, Anderlecht (BE); Georges Joseph Cornelius Hoornaert, Kessel-Lo (BE); Frans Josef Cornelius Compernolle, Herent (BE); Tomasz Kozlecki, Wroclaw (PL)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/915,202

(22) PCT Filed: May 24, 2006
(86) PCT No.: PCT/EP2006/062612

§ 371 Date: Nov. 21, 2007

(30) Foreign Application Priority Data
May 26, 2005 (EP) 05104511.0

(51) Int. Cl. A61K 31/4184; C07D 235/02; C07D 241/36; C07D 333/50; A61K 31/498; A61K 31/35; A61K 31/5377; A61P 25/00; A61K 31/403; A61K 31/381; C07D 311/02; C07D 209/56; C07D 413/12

(52) U.S. Cl. 514/232.8; 548/301.7; 544/342; 549/42; 549/383; 548/426; 544/142; 514/393; 514/250; 514/443; 514/453; 514/410

(57) ABSTRACT

This invention concerns novel substituted tetracyclic tetrahydrofuran, pyrrolidine and tetrahydrothiophene derivatives with binding affinities towards serotonin receptors, in particular 5-HT_{2A} and 5-HT_{2C} receptors, and towards dopamine receptors, in particular dopamine D2 receptors and with norepinephrine reuptake inhibition properties, pharmaceutical compositions comprising the compounds according to the invention, the use thereof as a medicine, in particular for the prevention and/or treatment of a range of psychiatric and neurological disorders, in particular certain psychotic, cardiovascular and gastrokinetic disorders and processes for their production.

The compounds according to the invention can be represented by general Formula (I)

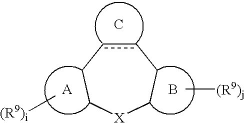

and comprises also the pharmaceutically acceptable acid or base addition salts thereof, the stereochemically isomeric forms thereof, the N-oxide form thereof and prodrugs thereof, wherein all substituents are defined as in Claim 1.

### FIELD OF THE INVENTION

This invention concerns novel substituted tetracyclic tetrahydrofuran, pyrrolidine and tetrahydrothiophene derivatives with binding affinities towards serotonin receptors, in particular 5-HT_{2A} and 5-HT_{2C} receptors, and towards dopamine receptors, in particular dopamine D2 receptors and with norepinephrine reuptake inhibition properties, pharmaceutical compositions comprising the compounds according to the invention, the use thereof as a medicine, in particular for the prevention and/or treatment of a range of psychiatric and neurological disorders, in particular certain psychotic, cardiovascular and gastrokinetic disorders and processes for their production.

### BACKGROUND PRIOR ART

WO 97/38991, published Oct. 23, 1997 (Janssen Pharmaceutica N.V.) discloses substituted tetracyclic tetrahydrofuran derivatives that may be used as therapeutic agents in the treatment or prevention of CNS disorders, cardiovascular disorders or gastrointestinal disorders. In particular, the compounds show affinity for the serotonin 5-HT₂ receptors, particularly for the 5-HT_{2A} and 5-HT_{2C}-receptors.

WO 99/19317, published Apr. 22, 1999 (Janssen Pharmaceutica N.V.) discloses substituted tetracyclic tetrahydrofuran derivatives with a specific halogen substitution pattern on the dibenzoazepine, dibenzooxepine, dibenzothiepine or dibenzosuberane ring. The compounds are useful in the treatment or prevention of CNS disorders, cardiovascular disorders or gastrointestinal disorders and show a faster onset of action over the compounds as disclosed in WO 97/38991.

Both WO 03/048146, published Jun. 12, 2003 (Janssen Pharmaceutica N.V.) and WO 03/048147, published Jun. 12, 2003 (Janssen Pharmaceutica N.V.) disclose processes for the preparation of each of the four diastereomers of trans-, respectively cis-fused 3,3a,8,12b-tetrahydro-2H-dibenzo[3,4:6,7]cyclohepta[1,2-b]furan derivatives in a stereochemically pure form from a single enantiomerically pure precursor. The compounds of WO 03/048146 show affinity for 5-HT₂ receptors, particularly for 5-HT_{2A} and 5-HT_{2C} receptors. The compounds of WO 03/048147 show affinity for the serotonin 5-HT_{2A}, 5-HT_{2C} and 5-HT₇ receptors, the H₁-receptors (pIC₅₀=7.15-7.89), D2 and/or D3 receptors and for the norepinephrine reuptake transporters (pIC₅₀=6.03-7.34). The compounds disclosed in the latter two publications do not contain a cyclic amine side chain.

WO 03/040122, published May 15, 2003 (Janssen Pharmaceutica N.V.) discloses mandelate salts of the compounds according to WO 97/38991 and WO 99/19317. Said salts were surprisingly found to be more stable at enhanced temperature and relative humidity than the compounds disclosed in WO 97/38991 and WO 99/19317.

### DESCRIPTION OF THE INVENTION

It is the object of the present invention to provide novel analogues of the tetracyclic tetrahydrofuran derivatives of WO 97/38991 and WO 99/19317, which differ from such derivatives in that they demonstrate in general more selectivity for the norepinephrine reuptake transporter than the 5-HT_{2A}, 5-HT_{2C} and dopamine D₂ receptors, resulting in compounds which have a more pronounced antidepressant effect in relation to their antipsychotic properties. The compounds of formula (I) below where the basic nitrogen atom at the C-2 position is embedded in a cyclic system demonstrate a potent antagonistic effect against the 5-HT_{2A}, 5-HT_{2C} and dopamine D₂ receptors.

This goal is achieved by the present novel compounds according to Formula (I):

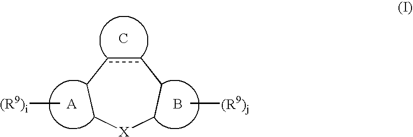

an N-oxide form, a pharmaceutically acceptable addition salt or a stereochemically isomeric form thereof, wherein:
the dotted line represents an optional bond;
i and j are integers, independently from each other, equal to zero, 1, 2, 3 or 4;
A and B are, each independently from each other, aryl or an heteroaryl radical selected from the group of furyl; thienyl; pyrrolyl; oxazolyl; thiazolyl; imidazolyl; isoxazolyl; isothiazolyl; oxadiazolyl; triazolyl; pyridinyl; pyridazinyl; pyrimidinyl; pyrazinyl; indolyl; indolizinyl; isoindolyl; benzofuryl; isobenzofuryl; benzothienyl; indazolyl; benzimidazolyl; benzthiazolyl; quinolizinyl; quinolinyl; isoquinolinyl; phthalazinyl; quinazolinyl; quinoxalinyl; chromenyl; naphthyridinyl and naphthalenyl;
each R⁹ is, independently from each other, selected from the group of hydrogen halo; cyano; hydroxy; carboxyl; nitro; amino; mono- or di(alkyl)amino; alkylcarbonylamino; aminosulfonyl; mono- or di(alkyl)aminosulfonyl; alkyl; alkyloxy; alkylcarbonyl and alkyloxycarbonyl;
X represents CR⁶R⁷, O, S, S(═O), S(═O)₂ or NR⁸; wherein:
   R⁶ and R⁷ each independently are selected from the group of hydrogen, hydroxy, alkyl and alkyloxy; or
   R⁶ and R⁷ taken together may form a radical selected from the group of methylene (═CH₂); mono- or di(cyano)methylene; a bivalent radical of formula —(CH₂)₂—, —(CH₂)₃—, —(CH₂)₄—, —(CH₂)₅—, —O(CH₂)₂O—, —O(CH₂)₃O—; or together with the carbon atom to which they are attached, a carbonyl;
   R⁸ is selected from the group of hydrogen; alkyl; alkylcarbonyl; arylcarbonyl; arylalkyl; arylalkylcarbonyl; alkylsulfonyl; aryl-sulfonyl and arylalkylsulfonyl;
C is a group of formula (c-1), (c-2), (c-3), (c-4) or (c-5)

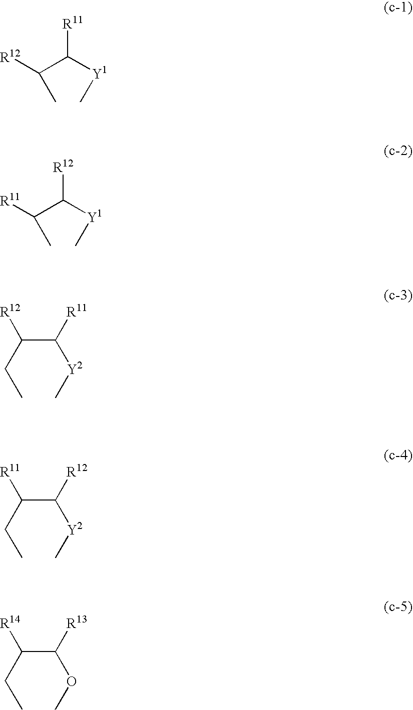

wherein:
Y¹ and Y² each independently are S; O; S(═O); S(═O)₂ or NR¹⁰; wherein R¹⁰ is selected from the group of hydrogen, cyano, alkyl, alkyloxyalkyl, formyl, alkylcarbonyl, alkyloxycarbonyl, alkyloxyalkylcarbonyl, arylcarbonyl, arylalkyl, arylalkylcarbonyl, alkylsulfonyl, arylsulfonyl and arylalkylsulfonyl;
R¹⁰ and R¹¹ may form together a bivalent radical (e-1) to (e-5);

—CH₂—NH—CH₂— (e-1)

—CH₂—NH—CH₂—CH₂— (e-2)

—CH₂CH₂—NH—CH₂— (e-3)

—CH═N—CH₂— (e-4)

—CH₂—N═CH₂— (e-5)
wherein optionally in each radical (e-1) to (e-5) one or more hydrogens are replaced by one or more substituents selected from alkyl, —O-alkyl, —S-alkyl ═O, ═S, ═S(═O) and ═S(═O)₂;
R¹² is hydrogen or alkyl;
R¹³, R¹⁴ each independently are hydrogen, hydroxy or oxo;
R¹¹ is a group of formula (d-1)

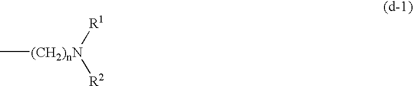

wherein:
n is zero, 1, 2, 3, 4, 5 or 6;
R¹ and R² each independently are hydrogen; alkyl; alkylcarbonyl; alkyloxyalkyl; alkylcarbonyloxyalkyl; alkyloxycarbonylalkyl;

arylalkyl; arylcarbonyl; alkyloxycarbonyl; aryloxycarbonyl;

arylalkylcarbonyl; alkyloxycarbonylalkylcarbonyl; mono- or di(alkyl)aminocarbonyl; mono- or di(aryl)aminocarbonyl; mono- or di(arylalkyl)aminocarbonyl; mono- or di(alkyloxycarbonylalkyl)aminocarbonyl; alkylsulphonyl; arylsulphonyl; arylalkylsulphonyl; mono- or di(alkyl)aminothiocarbonyl; mono- or di(aryl)aminothiocarbonyl; mono- or di(arylalkyl)-aminothiocarbonyl; mono-, di- or tri(alkyl)amidino; mono-, di- or tri(aryl)amidino and mono-, di- or tri(arylalkyl)amidino; or
R¹ and R² taken together with the nitrogen atom to which they are attached may form a radical of formula (a-1), (a-2), (a-3), (a-4), (a-5) or (a-6)

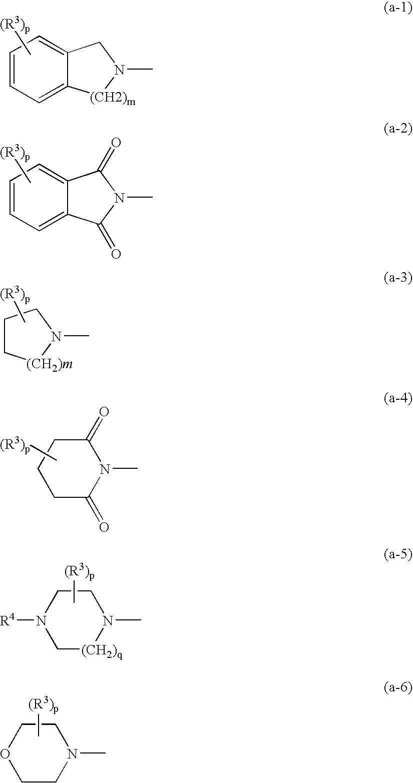

wherein
p is zero, 1, 2, 3 or 4;
q is 1 or 2;
m is zero, 1, 2, or 3
each R³ independently is selected from the group of hydrogen; halo; hydroxy; cyano; alkyl; alkyloxyalkyl; aryloxyalkyl; mono- or di-(alkyl)aminoalkyl; hydroxycarbonylalkyl; alkyloxycarbonylalkyl;

mono- or di(alkyl)aminocarbonylalkyl; mono- or di(aryl)aminocarbonylalkyl; mono- or di(alkyl)aminocarbonyloxyalkyl; alkyloxycarbonyloxyalkyl; arylaminocarbonyloxyalkyl; arylalkylaminocarbonyloxyalkyl; aryl; alkyloxy; aryloxy; alkylcarbonyloxy; arylcarbonyloxy; arylalkylcarbonyloxy; alkylcarbonyl; arylcarbonyl; aryloxycarbonyl; hydroxycarbonyl; alkyloxycarbonyl; alkylcarbonylamino; arylalkylcarbonylamino; arylcarbonylamino; alkyloxycarbonylamino; aminocarbonylamino; mono- or di(arylalkyl)aminocarbonylamino; alkylsulphonylalkylaminocarbonylamino; or two R³-radicals may form together a bivalent radical

—CR⁵R⁵—CR⁵R⁵—O— (b-1)

—O—CR⁵R⁵—CR⁵R⁵— (b-2)

—O—CR⁵R⁵—CR⁵R⁵—O— (b-3)

—O—CR⁵R⁵—CR⁵R⁵—CR⁵R⁵— (b-4)

—CR⁵R⁵—CR⁵R⁵—CR⁵R⁵—O— (b-5)

—O—CR⁵R⁵—CR⁵R⁵—CR⁵R⁵—O— (b-6)

—O—CR⁵R⁵—CR⁵R⁵—CR⁵R⁵—CR⁵R⁵— (b-7)

—CR⁵R⁵—CR⁵R⁵—CR⁵R⁵—CR⁵R⁵—O— (b-8)

—O—CR⁵R⁵—CR⁵R⁵—CR⁵R⁵—O— (b-9)
wherein R⁵ is selected from the group of hydrogen, halo, hydroxy, alkyloxy and alkyl;

R⁴ is selected from the group of hydrogen; alkyl; arylalkyl; alkyloxyalkyl; alkylcarbonyloxyalkyl; alkyloxycarbonylalkyl; arylcarbonylalkyl; alkylsulphonyloxyalkyl; aryloxyaryl; alkyloxycarbonylaryl; alkylcarbonyl; arylalkylcarbonyl; alkyloxycarbonylalkylcarbonyl; arylcarbonyl; alkyloxycarbonyl; aryloxycarbonyl; arylalkyloxycarbonyl; mono- or di(alkyl)aminocarbonyl; mono- or di(aryl)aminocarbonyl; mono- or di(arylalkyl)aminocarbonyl; mono- or di(alkyloxycarbonylalkyl)aminocarbonyl; alkyloxyalkylaminocarbonyl; mono-, di- or tri(alkyl)amidino; mono-, di- or tri(aryl)amidino; mono-, di- or tri(arylalkyl)-amidino; alkylsulphonyl; arylalkylsulphonyl or arylsulphonyl;
aryl is phenyl or naphthyl; each radical optionally substituted with 1, 2 or 3 substituents selected from the group of halo, nitro, cyano, hydroxy, alkyloxy or alkyl;
alkyl represents a straight or branched saturated hydrocarbon radical having from 1 to 10 carbon atoms, a cyclic saturated hydrocarbon radical having from 3 to 8 carbon atoms or a saturated hydrocarbon radical containing a straight or branched moiety having from 1 to 10 carbon atoms and a cyclic moiety having from 3 to 8 carbon atoms, optionally substituted with one or more halo, cyano, oxo, hydroxy, formyl, carboxyl or amino radicals; and
halo represents fluoro, chloro, bromo and iodo.

More in particular, the invention relates to a compound according to Formula (I), the pharmaceutically acceptable acid or base addition salts thereof, the stereochemically isomeric forms thereof, the N-oxide form thereof and a prodrug thereof, wherein A and B are each phenyl, optionally substituted with fluorine. Preferably, A is unsubstituted and B is substituted with fluor at the 11-position.

More in particular, the invention relates to a compound according to Formula (I), the pharmaceutically acceptable acid or base addition salts thereof, the stereochemically isomeric forms thereof, the N-oxide form thereof and a prodrug thereof, wherein X is CH₂ or O.

More in particular, the invention relates to a compound according to Formula (I), the pharmaceutically acceptable acid or base addition salts thereof, the stereochemically isomeric forms thereof, the N-oxide form thereof and a prodrug thereof, wherein C is a group of formula (c-1) or (c-2); wherein:
Y¹ is S; S(═O); S(═O)₂ or NR¹⁰; wherein R¹⁰ is selected from the group of hydrogen, cyano, alkyl, alkyloxyalkyl, formyl, alkylcarbonyl, alkyloxycarbonyl and alkyloxyalkylcarbonyl; or adjacent R¹⁰ and R¹¹ may form together a bivalent radical (e-1), (e-2) or (e-5); wherein optionally in each radical one or more hydrogens are replaced by one or more substituents selected from ═O, ═S, ═S(═O), alkyl and alkylthio; and
R¹² is hydrogen.

More in particular, the invention relates to a compound according to Formula (I), the pharmaceutically acceptable acid or base addition salts thereof, the stereochemically isomeric forms thereof, the N-oxide form thereof and a prodrug thereof, wherein C is a group of formula (c-3) or (c-4); wherein:
Y² is O or NH; and
R¹² is hydrogen.

More in particular, the invention relates to a compound according to Formula (I), the pharmaceutically acceptable acid or base addition salts thereof, the stereochemically isomeric forms thereof, the N-oxide form thereof and a prodrug thereof, wherein C is a group of formula (c-5); wherein:
R¹³ is hydrogen; and
R¹⁴ is hydroxy or oxo.

More in particular, the invention relates to a compound according to Formula (I), the pharmaceutically acceptable acid or base addition salts thereof, the stereochemically isomeric forms thereof, the N-oxide form thereof and a prodrug thereof, wherein (d-1) is defined as wherein:
n is zero or 1;
R¹ and R² each independently are hydrogen; alkyl or alkyloxycarbonylalkyl; or R¹ and R² taken together with the nitrogen atom to which they are attached may form a radical of formula (a-3), (a-5) or (a-6); wherein:
   p is zero or 1;
   q is 1;
   m is 1;
   each R³ independently is selected from the group of hydrogen and hydroxy; and
   R⁴ is alkyl.

More in particular, the invention relates to a compound according to the general Formula (I), the pharmaceutically acceptable acid or base addition salts thereof, the stereochemically isomeric forms thereof, the N-oxide form thereof and a prodrug thereof, wherein:
i and j are integers, independently from each other, equal to zero or 1;
A and B are, each independently from each other, phenyl, optionally substituted with fluor;
each R⁹ is, independently from each other, selected from the group of hydrogen and halo;
X is CH₂ and O;
C is a group of formula (c-1) or (c-2); wherein
   Y¹ is S; S(═O); S(═O)₂ or NR¹⁰; wherein R¹⁰ is selected from the group of hydrogen, cyano, alkyl, alkyloxyalkyl, formyl, alkylcarbonyl, alkyloxycarbonyl and alkyloxyalkylcarbonyl; or adjacent R¹⁰ and R¹¹ may form together a bivalent radical (e-1), (e-2) or (e-5); wherein optionally in each radicalone or more hydrogens are replaced by one or more substituents selected from ═O, ═S, ═S(═O), alkyl and alkylthio; and
   R¹² is hydrogen; or
C is a group of formula (c-3) or (c-4); wherein
   Y² is O or NH; and
   R¹² is hydrogen; or
C is a group of formula (c-5); wherein
   R¹³ is hydrogen; and
   R¹⁴ is hydroxy or oxo;
R¹¹ is a group of formula (d-1); wherein:
   n is zero or 1;
   R¹ and R² each independently are hydrogen; alkyl or alkyloxycarbonylalkyl; or R¹ and R² taken together with the nitrogen atom to which they are attached may form a radical of formula (a-3), (a-5) or (a-6); wherein:
      p is zero or 1;
      q is 1;
      m is 1;
      each R³ independently is selected from the group of hydrogen and hydroxy; and
      R⁴ is alkyl.

Preferably, alkyl is methyl, ethyl or propyl, optionally substituted with one or more halo, cyano, oxo, hydroxy, formyl, carboxyl or amino radicals. Preferably, alkyl is optionally substituted with hydroxy.

Preferably, aryl is phenyl, optionally substituted with 1, 2 or 3 substituents selected from the group of halo, nitro, cyano, hydroxy, alkyloxy or alkyl. Preferably, aryl is unsubstituted.

Preferably, halo is fluoro.

Preferred compounds are also those particular compounds according to the invention wherein the hydrogen atoms on carbon atoms 3a and 12b have a trans configuration and those having the (2α, 3aα, 12bβ) stereochemical configuration.

Preferred compounds are also those compounds according to the invention where the compounds are selected from the group of compounds:

Most preferred compounds are also those compounds according to the invention where the compounds are selected from the group of compounds defined by the compound numbers given in Tables 1 to 4.

### DETAILED DESCRIPTION OF THE INVENTION

In the framework of this application, alkyl is defined as a monovalent straight or branched saturated hydrocarbon radical having from 1 to 6 carbon atoms, for example methyl, ethyl, propyl, butyl, 1-methylpropyl, 1,1-dimethylethyl, pentyl and hexyl; alkyl further defines a monovalent cyclic saturated hydrocarbon radical having from 3 to 6 carbon atoms, for example cyclopropyl, methylcyclopropyl, cyclobutyl, cyclopentyl and cyclohexyl. The definition of alkyl also comprises an alkyl radical that is optionally substituted on one or more carbon atoms with one or more phenyl, halo, cyano, oxo, hydroxy, formyl and amino radicals, for example hydroxyalkyl, in particular hydroxymethyl and hydroxyethyl and polyhaloalkyl, in particular difluoromethyl and trifluoromethyl.

In the framework of this application, halo is generic to fluoro, chloro, bromo and iodo.

In the framework of this application, with “compounds according to the invention” is meant a compound according to the general Formula (I), the pharmaceutically acceptable acid or base addition salts thereof, the stereochemically isomeric forms thereof, the N-oxide form thereof and a prodrug thereof.

The pharmaceutically acceptable salts are defined to comprise the therapeutically active non-toxic acid addition salt forms that the compounds according to Formula (I) are able to form. Said salts can be obtained by treating the base form of the compounds according to Formula (I) with appropriate acids, for example inorganic acids, for example hydrohalic acid, in particular hydrochloric acid, hydrobromic acid, sulfuric acid, nitric acid and phosphoric acid; organic acids, for example acetic acid, hydroxyacetic acid, propanoic acid, lactic acid, pyruvic acid, oxalic acid, malonic acid, succinic acid, maleic acid, mandelic acid, fumaric acid, malic acid, tartaric acid, citric acid, methanesulfonic acid, ethanesulfonic acid, benzenesulfonic acid, p-toluenesulfonic acid, cyclamic acid, salicylic acid, p-aminosalicylic acid and pamoic acid.

The compounds according to Formula (I) containing acidic protons may also be converted into their therapeutically active non-toxic metal or amine addition salts forms by treatment with appropriate organic and inorganic bases. Appropriate base salts forms comprise, for example, the ammonium salts, the alkaline and earth alkaline metal salts, in particular lithium, sodium, potassium, magnesium and calcium salts, salts with organic bases, e.g. the benzathine, N-methyl-D-glucamine, hybramine salts, and salts with amino acids, for example arginine and lysine.

Conversely, said salts forms can be converted into the free forms by treatment with an appropriate base or acid.

The term addition salt as used in the framework of this application also comprises the solvates that the compounds according to Formula (I) as well as the salts thereof, are able to form. Such solvates are, for example, hydrates and alcoholates.

The N-oxide forms of the compounds according to Formula (I) are meant to comprise those compounds of Formula (I) wherein one or several nitrogen atoms are oxidized to the so-called N-oxide, particularly those N-oxides wherein one or more tertiary nitrogens (e.g of the piperazinyl or piperidinyl radical) are N-oxidized. Such N-oxides can easily be obtained by a skilled person without any inventive skills and they are obvious alternatives for the compounds according to Formula (I) since these compounds are metabolites, which are formed by oxidation in the human body upon uptake. As is generally known, oxidation is normally the first step involved in drug metabolism (Textbook of Organic Medicinal and Pharmaceutical Chemistry, 1977, pages 70-75). As is also generally known, the metabolite form of a compound can also be administered to a human instead of the compound per se, with much the same effects.

The compounds according to the invention possess at least 1 oxydizable nitrogen (tertiary amines moiety). It is therefore highly likely that N-oxides are to form in the human metabolism.

The compounds of Formula (I) may be converted to the corresponding N-oxide forms following art-known procedures for converting a trivalent nitrogen into its N-oxide form. Said N-oxidation reaction may generally be carried out by reacting the starting material of Formula (I) with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzene-carboperoxoic acid, peroxoalkanoic acids, e.g. peroxoacetic acid, alkylhydroperoxides, e.g. tert-butyl hydroperoxide. Suitable solvents are, for example, water, lower alkanols, e.g. ethanol and the like, hydrocarbons, e.g. toluene, ketones, e.g. 2-butanone, halogenated hydrocarbons, e.g. dichloromethane, and mixtures of such solvents.

The term “stereochemically isomeric forms” as used hereinbefore defines all the possible isomeric forms that the compounds of Formula (I) may possess. Unless otherwise mentioned or indicated, the chemical designation of compounds denotes the mixture of all possible stereochemically isomeric forms, said mixtures containing all diastereomers and enantiomers of the basic molecular structure. More in particular, stereogenic centers may have the R— or S-configuration; substituents on bivalent cyclic (partially) saturated radicals may have either the cis- or trans-configuration. Compounds encompassing double bonds can have an E or Z-stereochemistry at said double bond. Stereochemically isomeric forms of the compounds of Formula (I) are obviously intended to be embraced within the scope of this invention.

Following CAS nomenclature conventions, when two stereogenic centers of known absolute configuration are present in a molecule, an R or S descriptor is assigned (based on Cahn-Ingold-Prelog sequence rule) to the lowest-numbered chiral center, the reference center. R* and S* each indicate optically pure stereogenic centers with undetermined absolute configuration. If “α” and “β” are used: the position of the highest priority substituent on the asymmetric carbon atom in the ring system having the lowest ring number, is arbitrarily always in the “α” position of the mean plane determined by the ring system. The position of the highest priority substituent on the other asymmetric carbon atom in the ring system (hydrogen atom in compounds according to Formula (I)) relative to the position of the highest priority substituent on the reference atom is denominated “α”, if it is on the same side of the mean plane determined by the ring system, or “β”, if it is on the other side of the mean plane determined by the ring system.

The numbering of the tetracyclic ring-systems present in the compounds of Formula (I-a) and (I-b) when A and B are phenyl, as defined by Chemical Abstracts nomenclature is shown below.

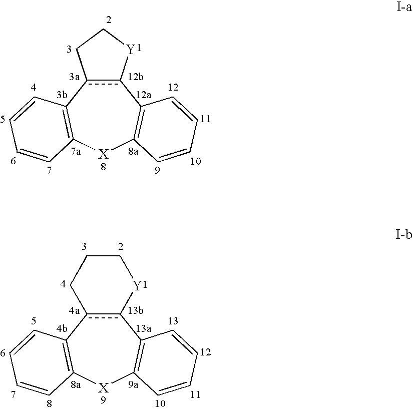

The compounds of Formula (I-a) and (I-b) have at least two asymmetric centers at respectively carbon atom 2 and 3. Said asymmetric center and any other asymmetric center, which may be present (e.g. at atom 8 in (I-a) or 9 in (I-b)), are indicated by the descriptors R and S. When e.g. a monocyanomethylene moiety is present in the compounds of Formula (I-a) at position 8, said moiety may have the E- or Z-configuration.

The invention also comprises derivative compounds (usually called “pro-drugs”) of the pharmacologically active compounds according to the invention, which are degraded in vivo to yield the compounds according to the invention. Pro-drugs are usually (but not always) of lower potency at the target receptor than the compounds to which they are degraded. Pro-drugs are particularly useful when the desired compound has chemical or physical properties that make its administration difficult or inefficient. For example, the desired compound may be only poorly soluble, it may be poorly transported across the mucosal epithelium, or it may have an undesirably short plasma half-life. Further discussion on pro-drugs may be found in Stella, V. J. et al., “Prodrugs”, Drug Delivery Systems, 1985, pp. 112-176, and Drugs, 1985, 29, pp. 455-473.

Prodrugs forms of the pharmacologically-active compounds according to the invention will generally be compounds according to Formula (I), the pharmaceutically acceptable acid or base addition salts thereof, the stereochemically isomeric forms thereof and the N-oxide form thereof, having an acid group which is esterified or amidated. Included in such esterified acid groups are groups of the Formula —COOR^{x}, where R^{x} is a C₁₋₆alkyl, phenyl, benzyl or one of the following groups:

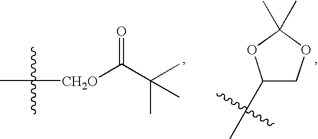

Amidated groups include groups of the Formula —CONR^{y}R^{z}, wherein R^{y} is H, C₁₋₆alkyl, phenyl or benzyl and R^{z} is —OH, H, C₁₋₆alkyl, phenyl or benzyl. Compounds according to the invention having an amino group may be derivatised with a ketone or an aldehyde such as formaldehyde to form a Mannich base. This base will hydrolyze with first order kinetics in aqueous solution.

The compounds of Formula (I) as prepared in the processes described below may be synthesized in the form of racemic mixtures of enantiomers that can be separated from one another following art-known resolution procedures. The racemic compounds of Formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

### Pharmacology

The compounds of the present invention show affinity for 5-HT₂ receptors, particularly for 5-HT_{2A} and 5-HT_{2C} receptors (nomenclature as described by D. Hoyer in “Serotonin (5-HT) in neurologic and psychiatric disorders” edited by M. D. Ferrari and published in 1994 by the Boerhaave Commission of the University of Leiden) and affinity for the D2 receptor as well as norepinephrine reuptake inhibition activity. The serotonin antagonistic properties of the present compounds may be demonstrated by their inhibitory effect in the “5-hydroxytryptophan Test on Rats” which is described in Drug Dev. Res., 13, 237-244 (1988).

In view of their capability to block 5-HT₂ receptors, and in particular to block 5-HT_{2A} and 5-HT_{2C} receptors, as well as the D2 receptor and by also effecting the norepinephrine reuptake inhibition activity, the compounds according to the invention are useful as a medicine, in particular in the prophylactic and therapeutic treatment of conditions mediated through either of these receptors.

The invention therefore relates to a compound according to the general Formula (I), the pharmaceutically acceptable acid or base addition salts thereof, the stereochemically isomeric forms thereof, the N-oxide form thereof and prodrugs thereof, for use as a medicine.

The invention also relates to the use of a compound according to the general Formula (I), the pharmaceutically acceptable acid or base addition salts thereof, the stereochemically isomeric forms thereof, the N-oxide form thereof and prodrugs thereof for the manufacture of a medicament for treating, either prophylactic or therapeutic or both, conditions mediated through the 5-HT₂, and D2 receptor, as well as the through norepinephrine reuptake inhibition.

In view of these pharmacological and physicochemical properties, the compounds of Formula (I) are useful as therapeutic agents in the treatment or the prevention of central nervous system disorders like anxiety, depression and mild depression, bipolar disorders, sleep- and sexual disorders, psychosis, borderline psychosis, schizophrenia, migraine, personality disorders or obsessive-compulsive disorders, social phobias or panic attacks, organic mental disorders, mental disorders in children such as ADHD, aggression, memory disorders and attitude disorders in older people, addiction, obesity, bulimia and similar disorders. In particular, the present compounds may be used as anxiolytics, antidepressants, antipsychotics, anti-schizophrenia agents, anti-migraine agents and as agents having the potential to overrule the addictive properties of drugs of abuse.

The compounds of Formula (I) may also be used as therapeutic agents in the treatment of motoric disorders. It may be advantageous to use the present compounds in combination with classical therapeutic agents for such disorders.

The compounds of Formula (I) may also serve in the treatment or the prevention of damage to the nervous system caused by trauma, stroke, neurodegenerative illnesses and the like; cardiovascular disorders like high blood pressure, thrombosis, stroke, and the like; and gastrointestinal disorders like dysfunction of the motility of the gastrointestinal system and the like.

In view of the above uses of the compounds of Formula (I), it follows that the present invention also provides a method of treating warm-blooded animals suffering from such diseases, said method comprising the systemic administration of a therapeutic amount of a compound of Formula (I) effective in treating the above described disorders, in particular, in treating anxiety, psychosis, depression, migraine and addictive properties of drugs of abuse.

The present invention thus also relates to compounds of Formula (I) as defined hereinabove for use as a medicine, in particular, the compounds of Formula (I) may be used for the manufacture of a medicament for treating anxiety, psychosis, depression, migraine and addictive properties of drugs of abuse.

Those of skill in the treatment of such diseases could determine the effective therapeutic daily amount from the test results presented hereinafter. An effective therapeutic daily amount would be from about 0.01 mg/kg to about 10 mg/kg body weight, more preferably from about 0.05 mg/kg to about 1 mg/kg body weight.

The invention also relates to a pharmaceutical composition comprising a pharmaceutically acceptable carrier and, as active ingredient, a therapeutically effective amount of a compound according to the invention, in particular a compound according to Formula (I), the pharmaceutically acceptable acid or base addition salts thereof, the stereochemically isomeric forms thereof, the N-oxide form thereof and a prodrug thereof.

The compounds according to the invention, in particular the compounds according to Formula (I), the pharmaceutically acceptable acid or base addition salts thereof, the stereochemically isomeric forms thereof, the N-oxide form thereof and the prodrugs thereof, or any subgroup or combination thereof may be Formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, in particular, for administration orally, rectally, percutaneously, by parenteral injection or by inhalation. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

It is especially advantageous to Formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

Since the compounds according to the invention are potent orally administrable compounds, pharmaceutical compositions comprising said compounds for administration orally are especially advantageous.

In order to enhance the solubility and/or the stability of the compounds of Formula (I) in pharmaceutical compositions, it can be advantageous to employ α-, β- or γ-cyclodextrins or their derivatives, in particular hydroxyalkyl substituted cyclodextrins, e.g. 2-hydroxypropyl-β-cyclodextrin. Also co-solvents such as alcohols may improve the solubility and/or the stability of the compounds according to the invention in pharmaceutical compositions.

### Preparation

Suitable preparation methods for the compounds of the invention are described below: The following abbreviations are used thoughout the text:

| | |
|---|---|
| APCI | Atmospheric Pressure Chemical Ionization |
| AcOH | acetic acid |
| Ac₂O | Acetic anhydride |
| AcSH | thioacetic acid |
| Bu | n-butyl |
| Boc | tert-butyloxycarbonyl |
| Cbz- | 4-carboxybenzoyl (e.g. CbzCl) |
| Celite ® | diatomaceous earth from Celite Corporation |
| CI | Chemical Ionization |
| CSA | camphorsulfonic acid |
| DBU | 1,8-diazabicyclo[5,4,0]undec-7-ene |
| DIAD | diisopropyl azodicarboxylate |
| DHP | 2,3-dihydropirane |
| DMAP | N,N-4-dimethylaminopyridine |
| DMF | N,N-dimethylformamide |
| DOWEX ® | ion exchange resin from the company DOW |
| DPPA | diphenyl phosphoryl azide |
| EEDQ | 2-ethoxy-1-ethoxycarbonyl-1,2-dihydroquinoline |
| EI | Electron Ionization |
| Et | ethyl |
| Et₃N | triethylamine |
| EtOH | ethanol |
| Et₂O | diethylether |
| EtOAc | ethyl acetate |
| HFIP | hexafluoroisopropanol |
| i-PrOH | isopropanol |
| IPy₂BF₄ | bis(pyridine)iodonium tetrafluoroborate |
| t-BuOK | potassium salt of 2-methyl-2-propanol |
| mCPBA | m-chloroperoxybenzoic acid |
| Ns | Nosyl (e.g. o-nitrophenylsulfonyl chloride: NsCl) |
| Me | methyl |
| MeOH | methanol |
| Ms | mesyl (e.g. mesyl chloride: MsCl) |
| PCC | pyridinium chlorochromate |
| PPh₃ | triphenylphosphine |
| TFA | trifluoroacetic acid |
| THF | tetrahydrofuran |
| THP | tetrahydropyranyl |
| Tr | trityl (e.g. triphenylmethyl chloride: TrCl) |
| Ts | tosyl (e.g. 4-toluenesulfonyl chloride: TsCl) |

The following reaction methods A to D illustrate the preparation of compounds of formula (I) in which C is a group of formula (c-1) in which Y¹ is NH, R¹¹ is a group of formula (d-1) and R¹² is hydrogen, represented by formulae (I-a) and (I-b) below:

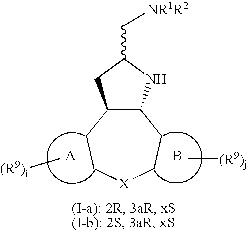

wherein x is 12b if A and B are each six-membered rings, such as phenyl

### Method A: Preparation of Pyrrolidine Derivatives

### Method A1: Synthesis of (2R,3aR,xS)-intermediate compounds

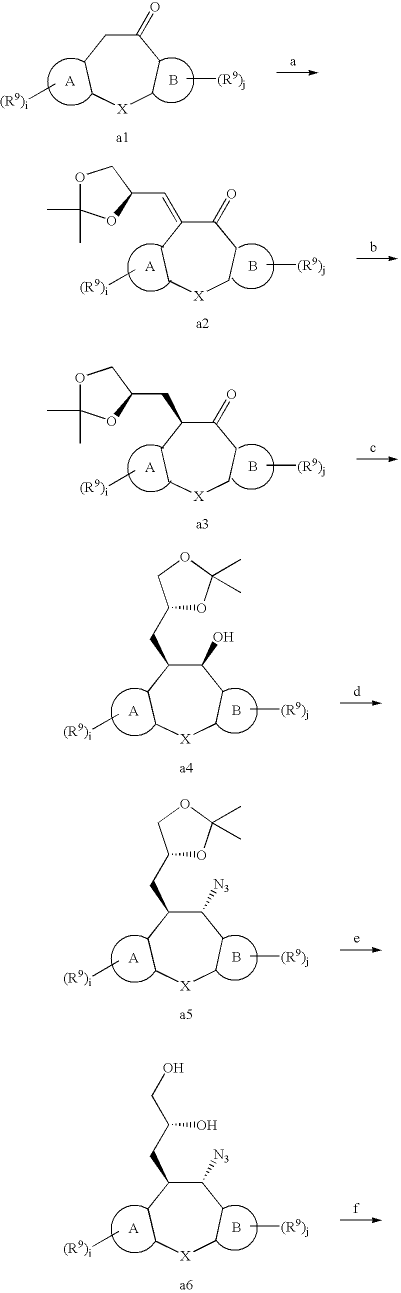

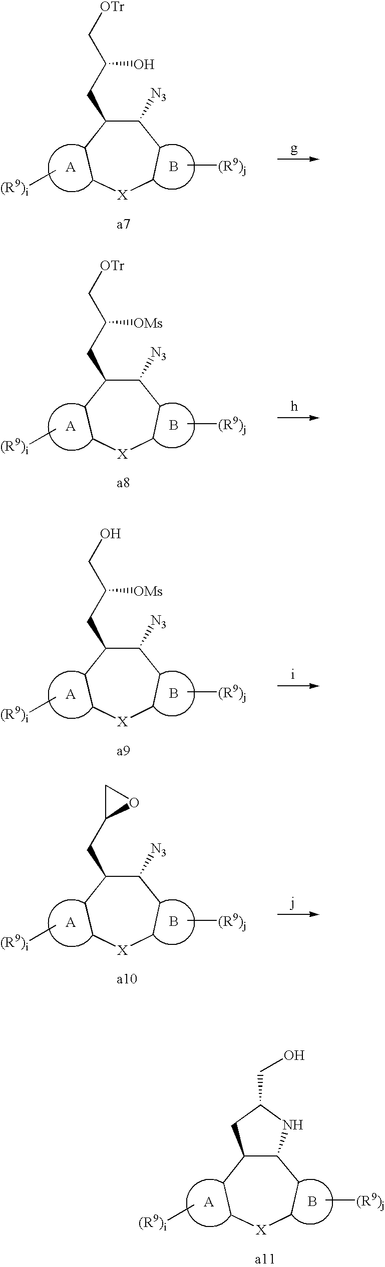

### Method A2: Synthesis of (2S3aR,xS)-Intermediate and Final Compounds

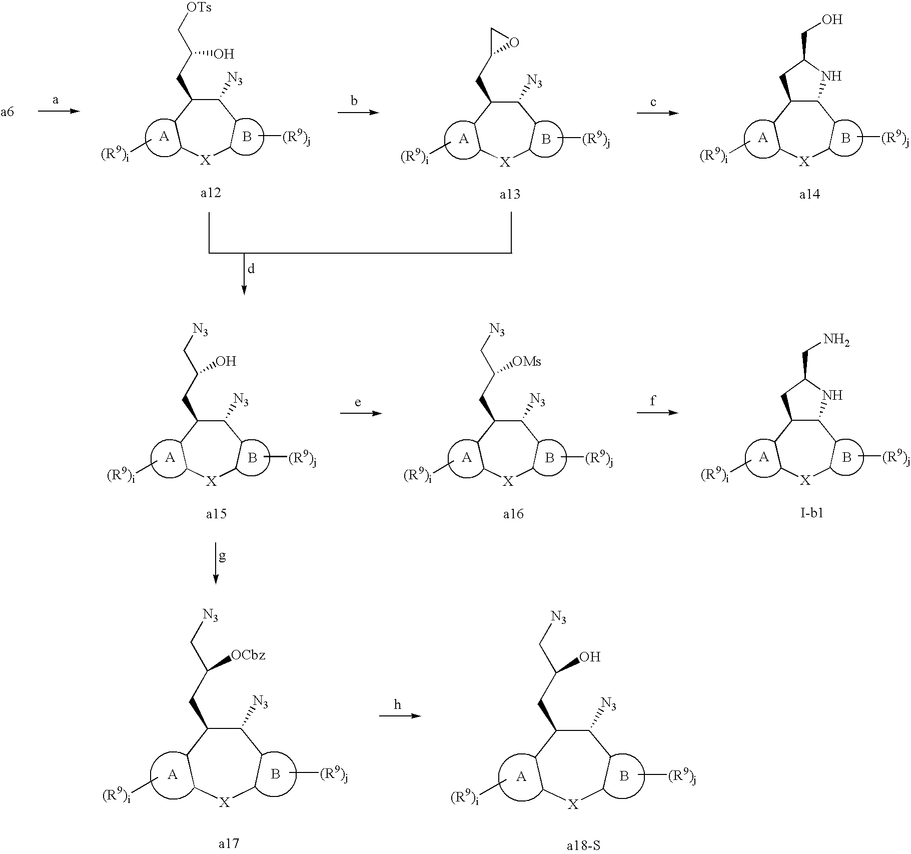

### Method A3: Synthesis of (2R,3aR,xS)- and (2S,3aR,xS)-Final Compounds

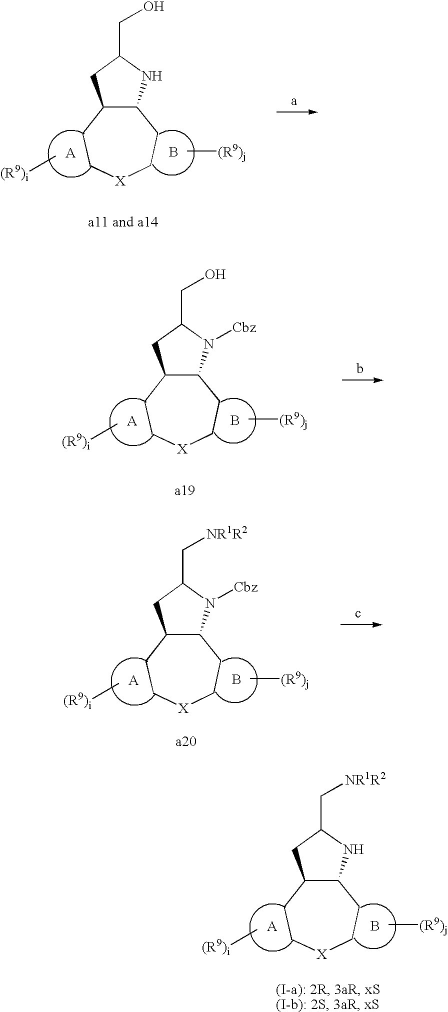

### Method B: Synthesis of (2R,3aR,xS)- and (2S,3aR,xS)-Final Compounds

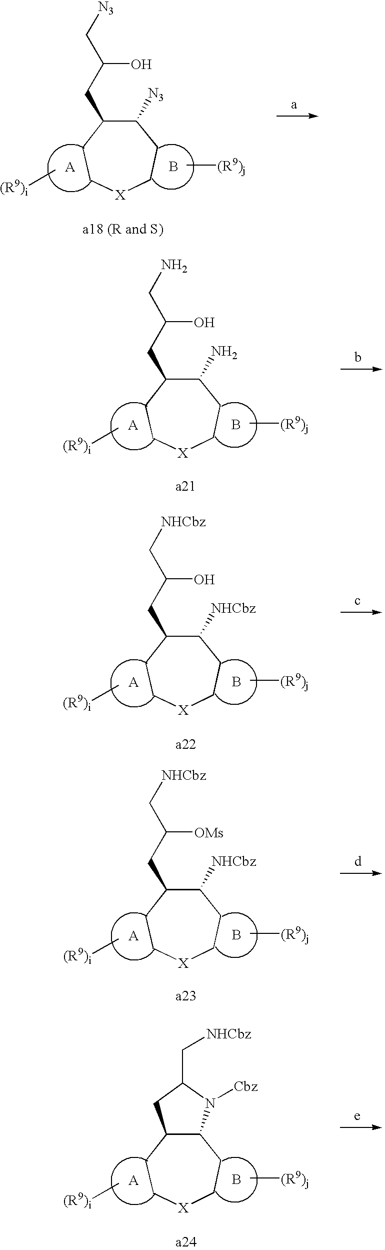

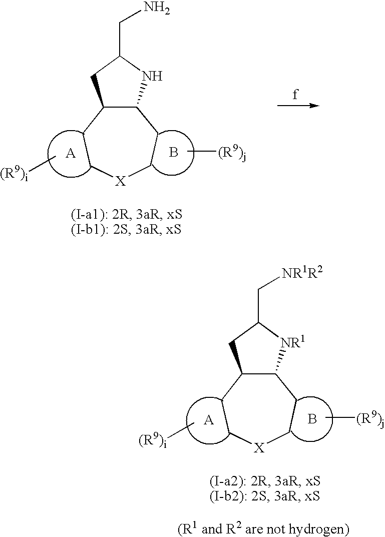

### Method C: Synthesis of (2RS,3aR*,xS*)-Final Compounds

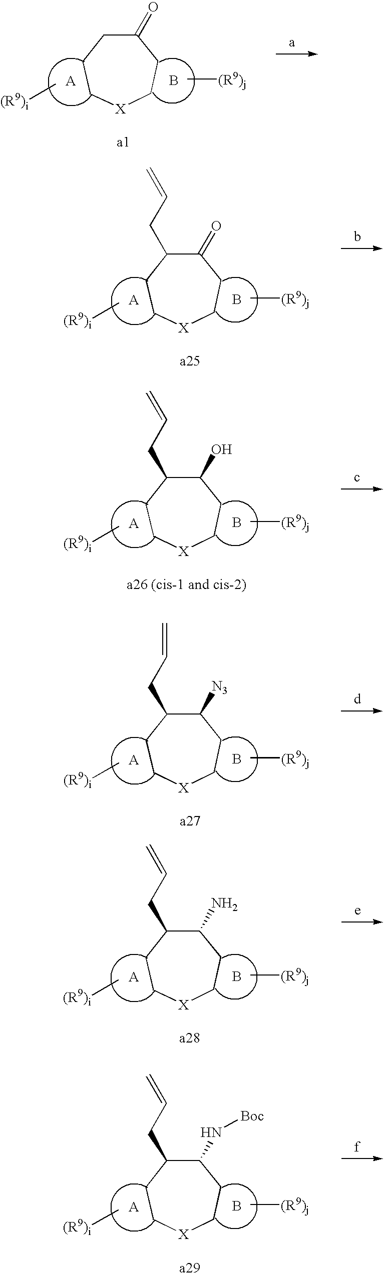

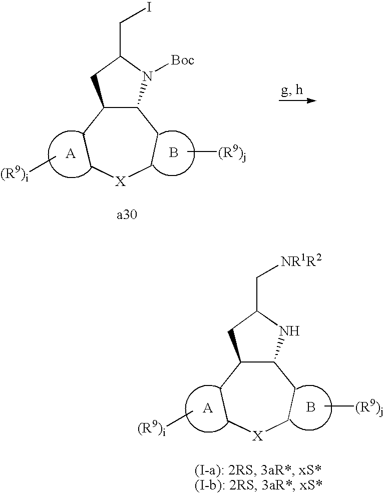

### Method D: Synthesis of (2RS,3aR,xS)-Final Compounds

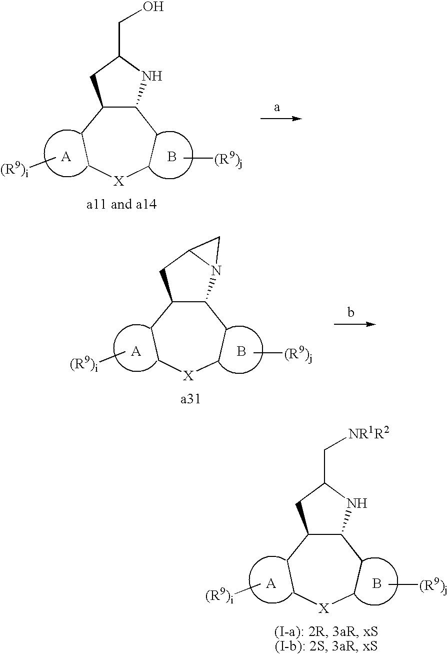

### Method E: Preparation of Pyrroloimidazole Derivatives

The following method illustrates the preparation of compounds of formula (I) in which C is a group of formula (c-1) in which R¹¹ and R¹⁰ form a bivalent radical, represented by formulae (II-a), (I-b) and (II-c) below.

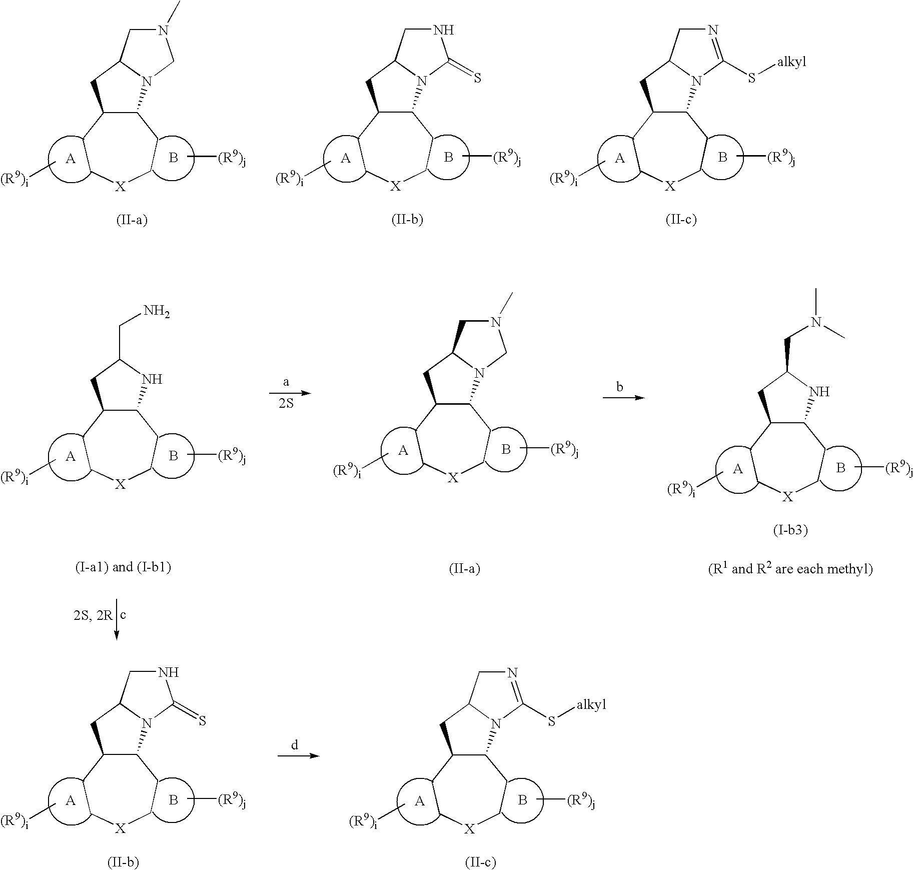

### Method F: Preparation of Pyrrolopiperazine Derivatives

The following methods illustrate the preparation of compounds of formula (I) in which C is a group of formula (c-1) in which R¹¹ and R¹⁰ form a condensed piperazine residue, represented by formula (III) below in which R¹³ is hydrogen or alkyl and the piperazine ring has the S configuration (Method F1) or the R configuration (Method F2).

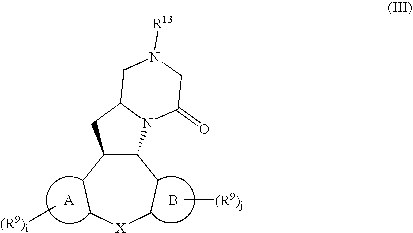

### Method F1

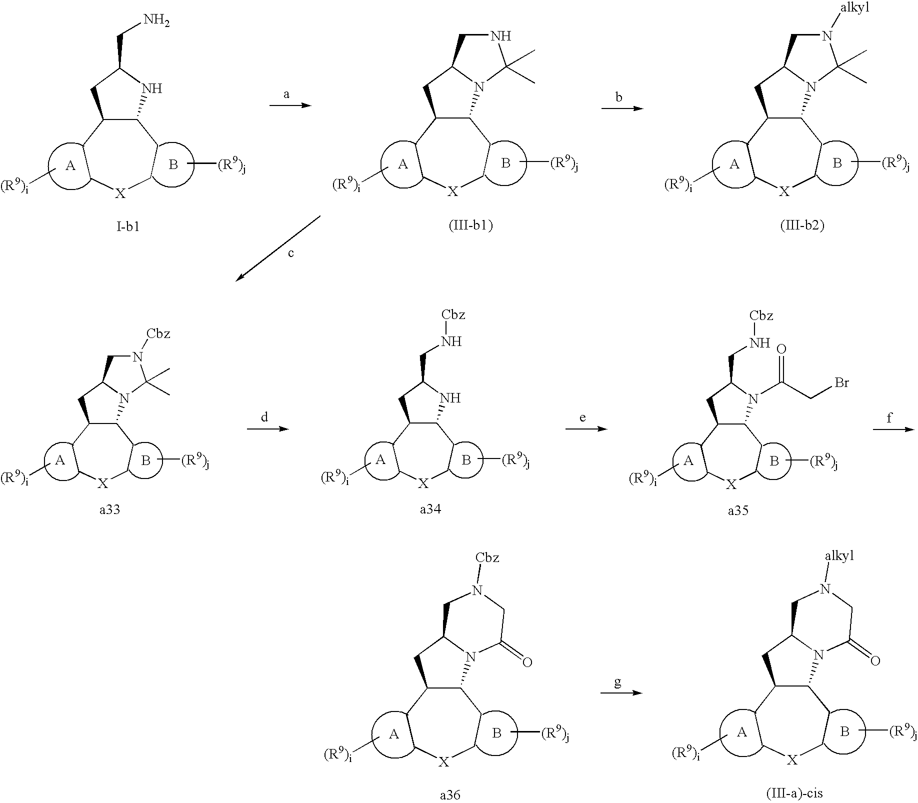

### Method F2

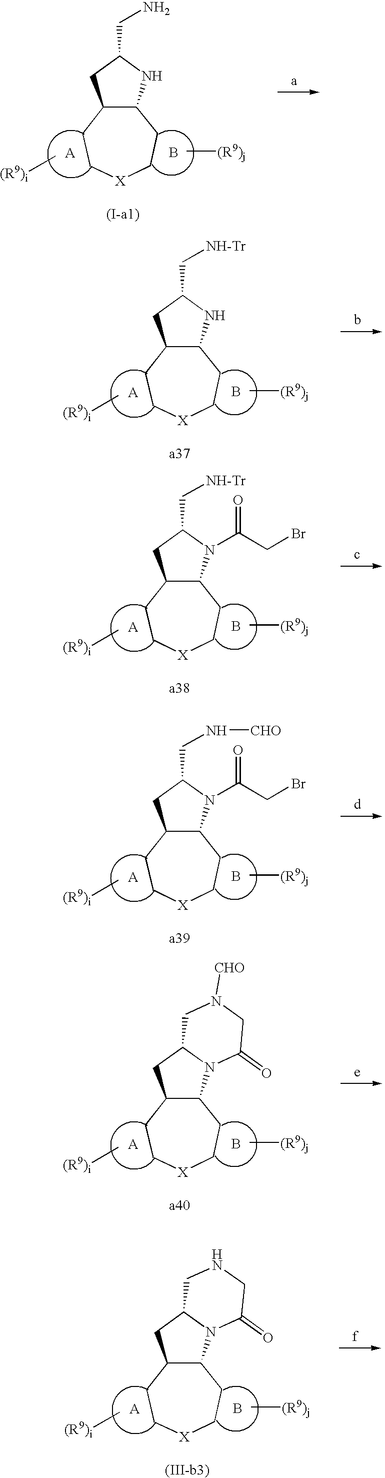

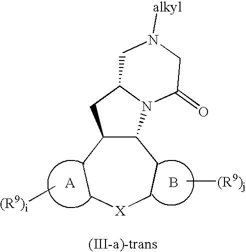

### Method G: Preparation of 8,8-substituted Pyrrolidinederivatives

The following method illustrates the preparation of compounds of formula (I) in which C is a group of formula (c-1) wherein Y¹ is NH, R¹¹ is a group of formula (d-1) and R¹² is hydrogen and X is a CR⁶R⁷ group with R⁶ and R⁷ other than a hydrogen group, represented by formula (IV) below.

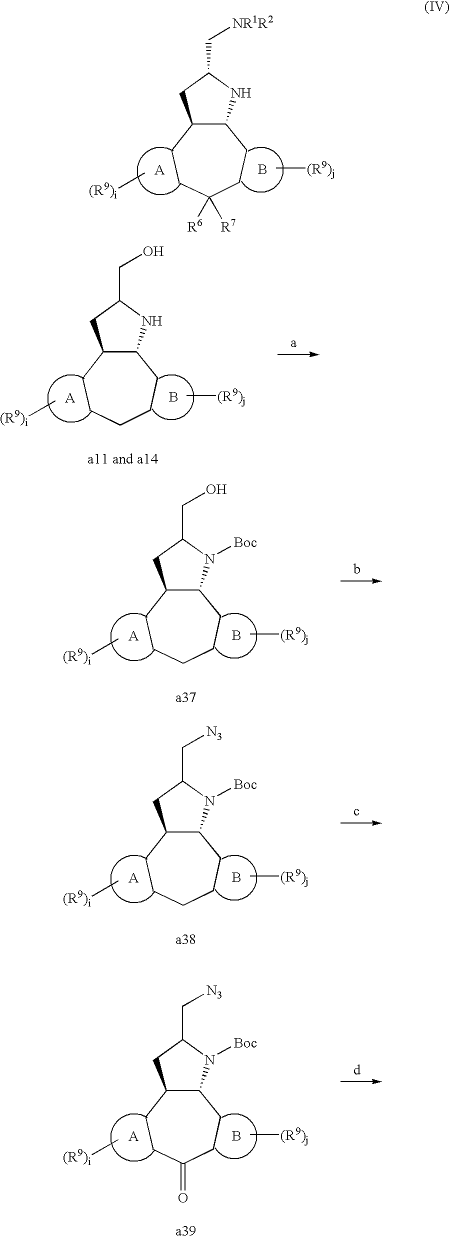

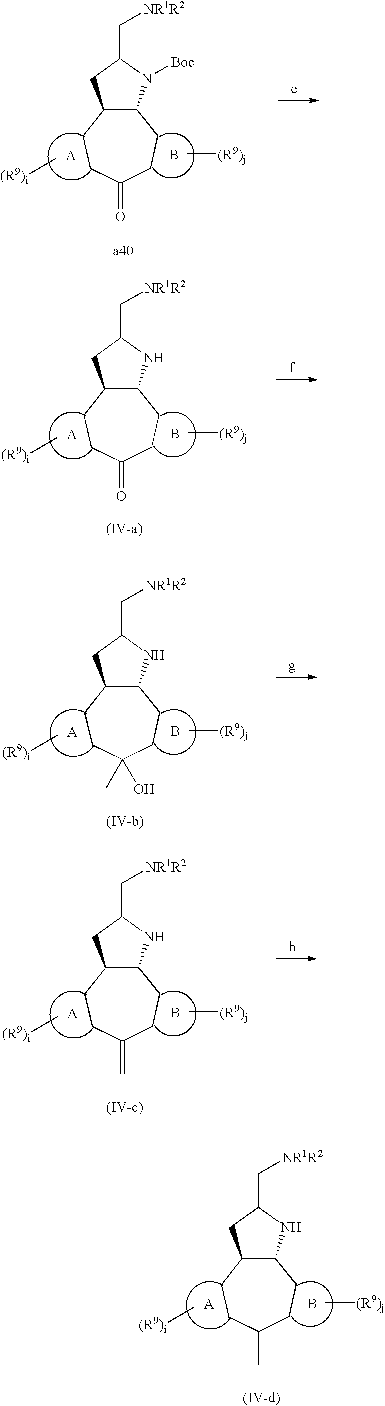

All compounds of formula (IV) have either the 2R- or 2S-configuration, depending on the starting compound a11 or a14.

### Method H: Preparation of 3-substituted Pyrrolidine Derivatives

The following method illustrates the preparation of compounds of formula (I) in which C is a group of formula (c-2), wherein Y¹ is NH and R¹¹ is a group of formula (d-1) and R¹² is hydrogen, represented by formula (V) below.

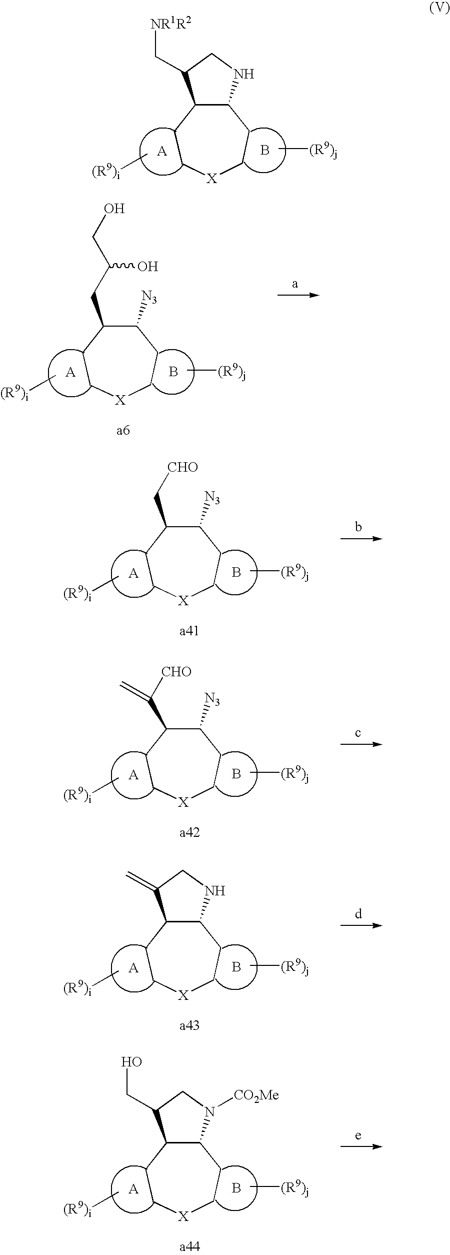

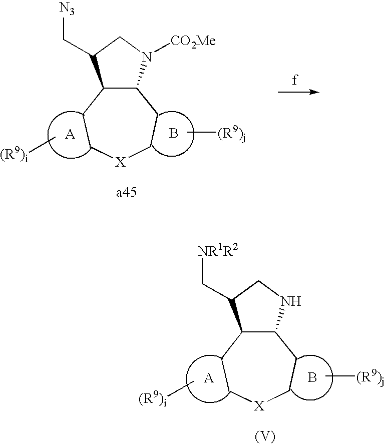

### Method I: Preparation of Tetrahydrofurane-3-substituted Derivatives

The following method illustrates the preparation of compounds of formula (I) in which C is a group of formula (c-3), R¹¹ is a group of formula (d-1) and R¹² is hydrogen, represented by formula (VI) below.

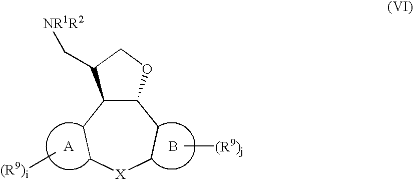

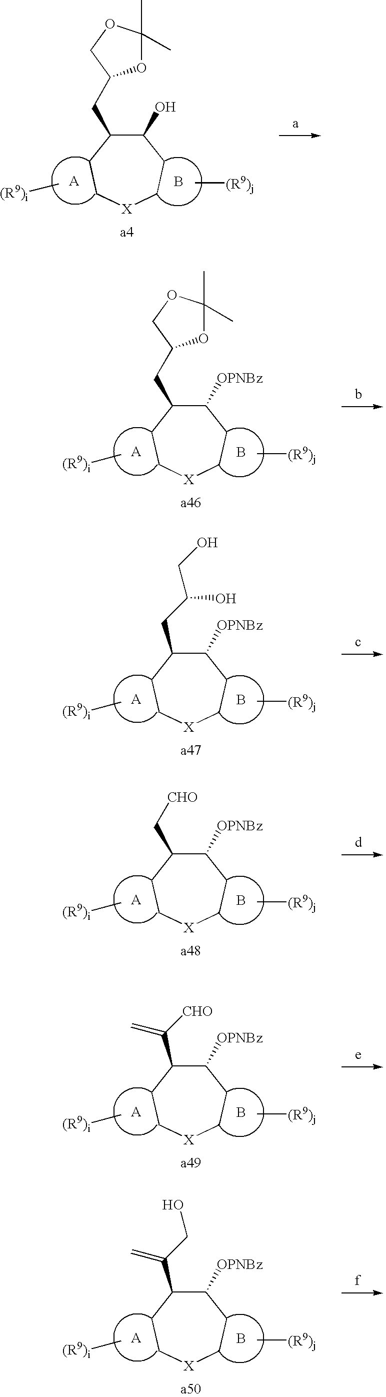

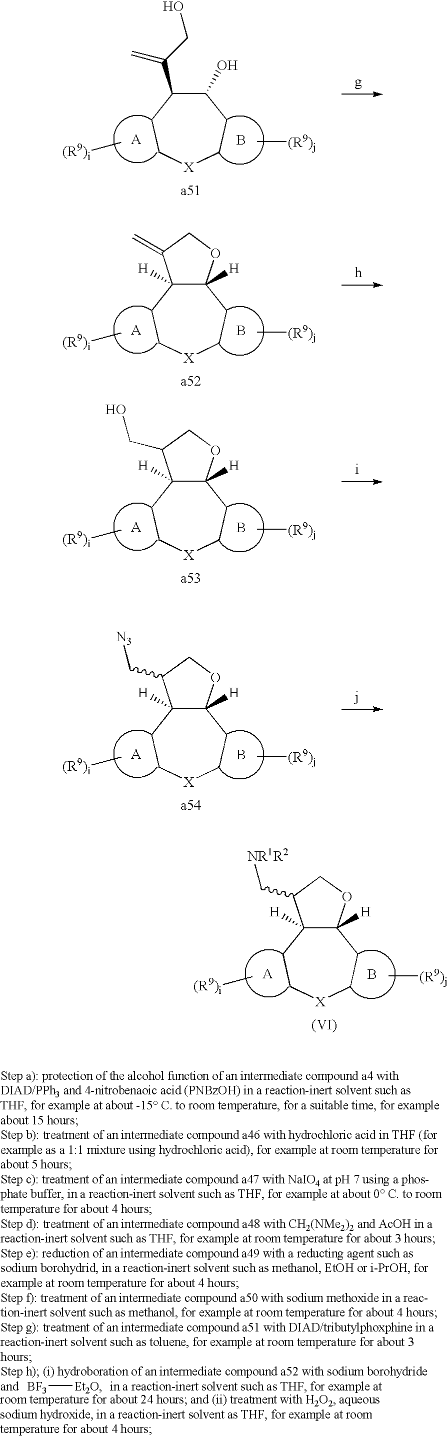

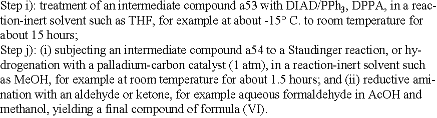

### Method J: Preparation of 3-substituted Tetrahydropyran Derivatives

The following method illustrates the preparation of compounds of formula (I) in which C is a group of formula (c-5), Y² is O, R¹² is hydrogen and R¹⁴ is a group of formula (d-1), represented by formula (VII) below. The compound can be either cis (Method J1) or trans (Method J2) with respect to the oxygen.

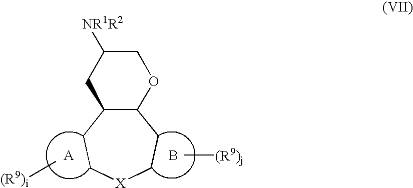

### Method J1 (cis)

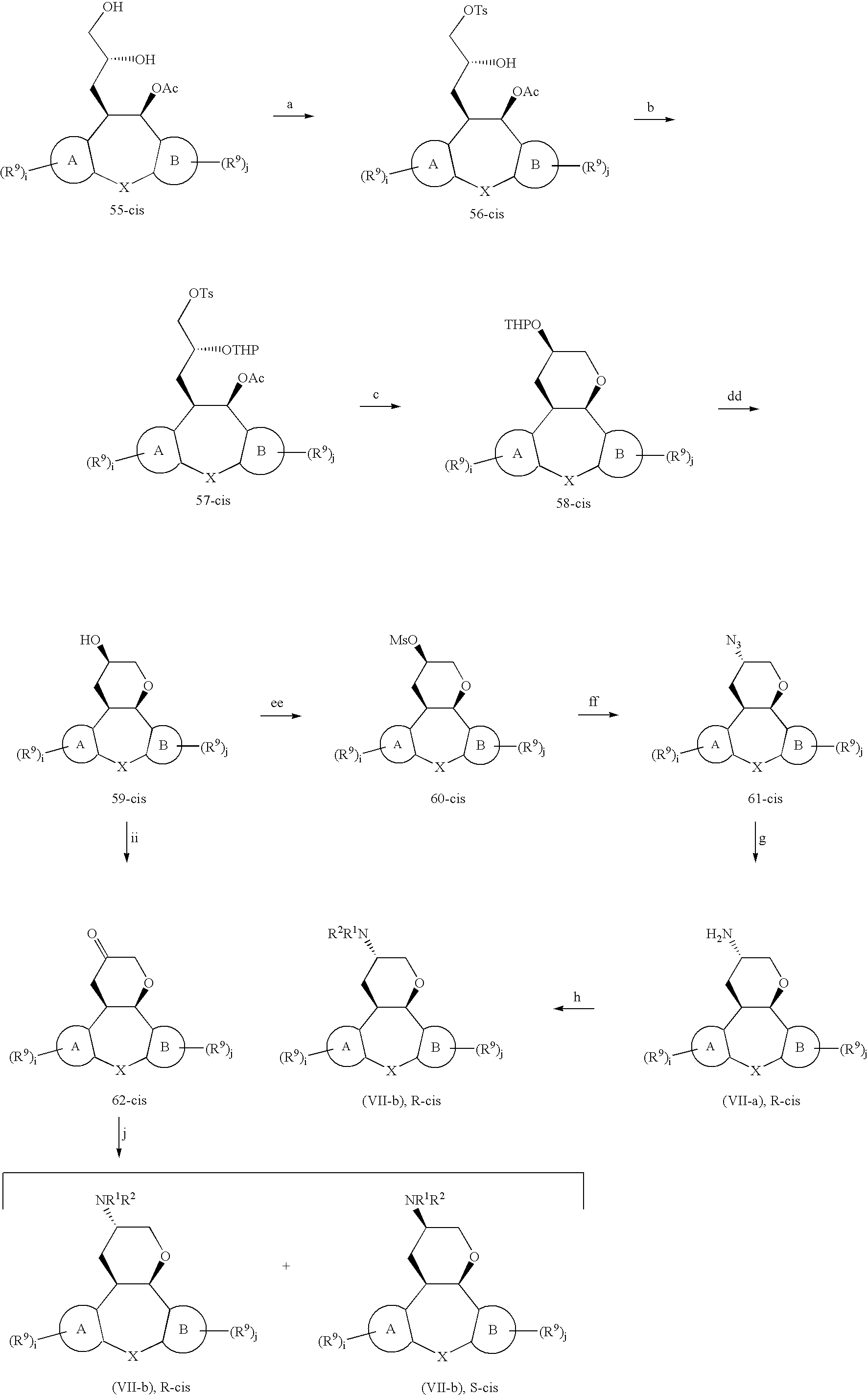

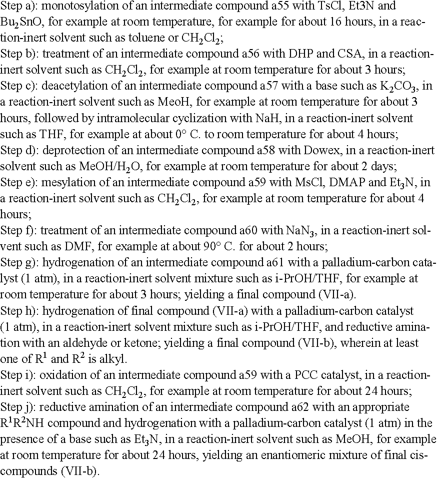

### Method J2 (trans)

The method J1 can also be applied to the trans-epimer of intermediate compound a55, leading to trans-final compounds of formulae (VII-a) and (VII-b) below.

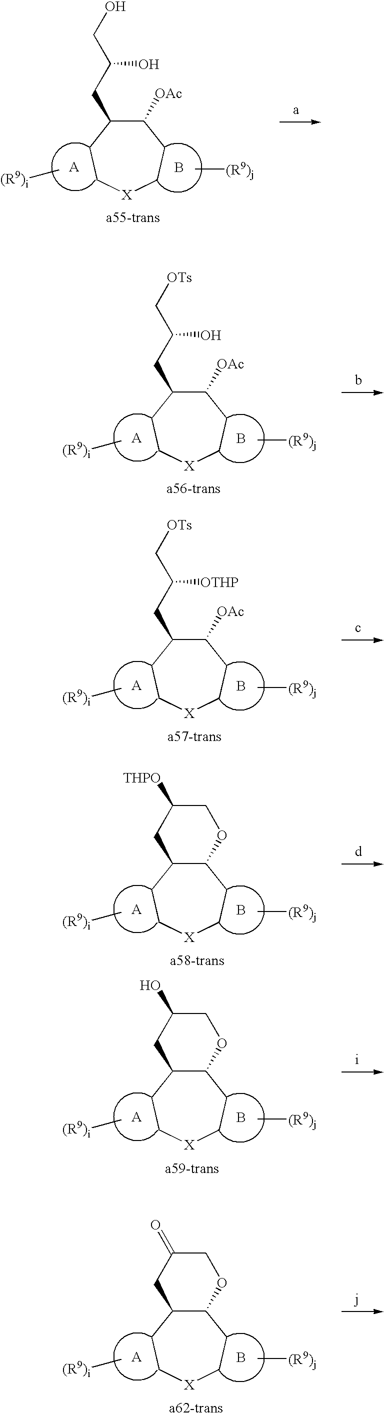

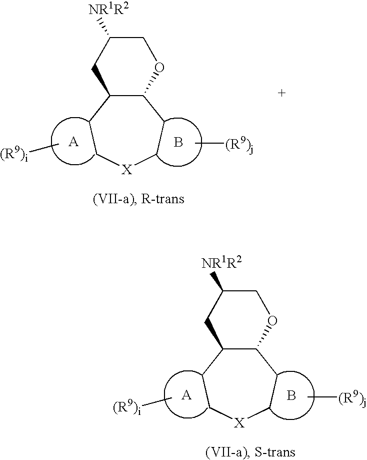

### Method K: Preparation of 4-substituted Tetrahydropyran-Derivatives

The following method illustrates the preparation of compounds of formula (I) in which C is a group of formula (c-4), y² is O, R¹¹ is a group of formula (d-1) and R¹² is hydrogen, represented by formula (VIII) below.

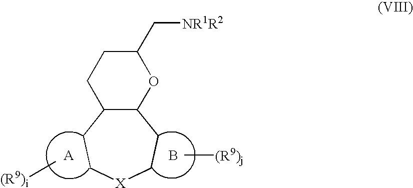

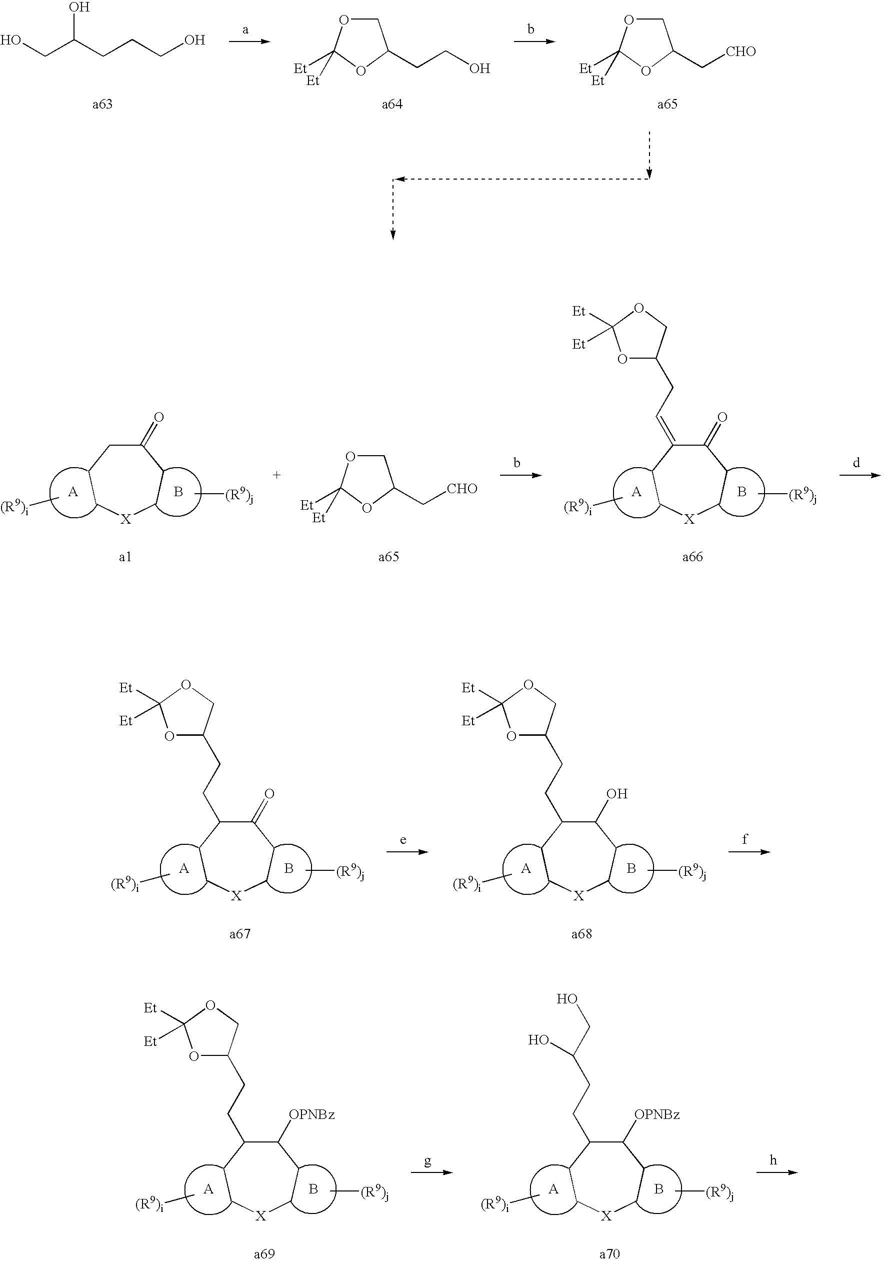

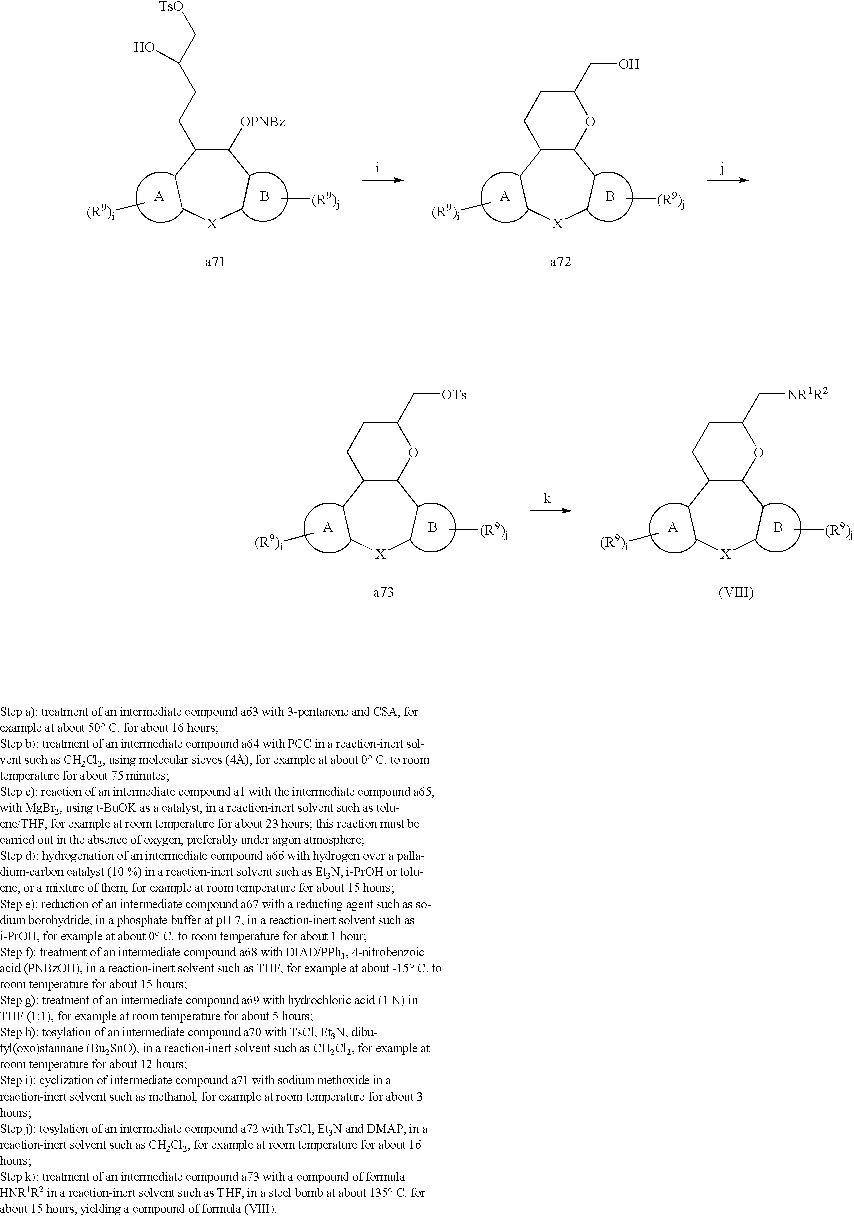

### Method L: Preparation of Tetrahydrothiophene-2-substituted Derivatives

The following methods illustrates the preparation of compounds of formula (I) in which C is a group of formula (c-1) wherein Y¹ is SOₙ and R¹¹ is a group of formula (d-1) and R¹² is hydrogen, represented by formulae (IX), (X) and (XI) below.

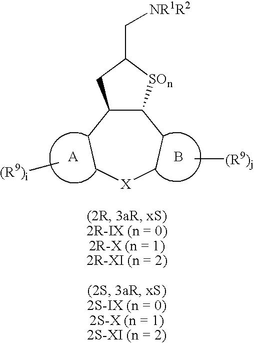

where x is 12b if A and B is a six-membered ring, such as phenyl

### Method L1: Synthesis of (2R,3aR,xS)-tetrahydrothiophene Intermediate Compounds

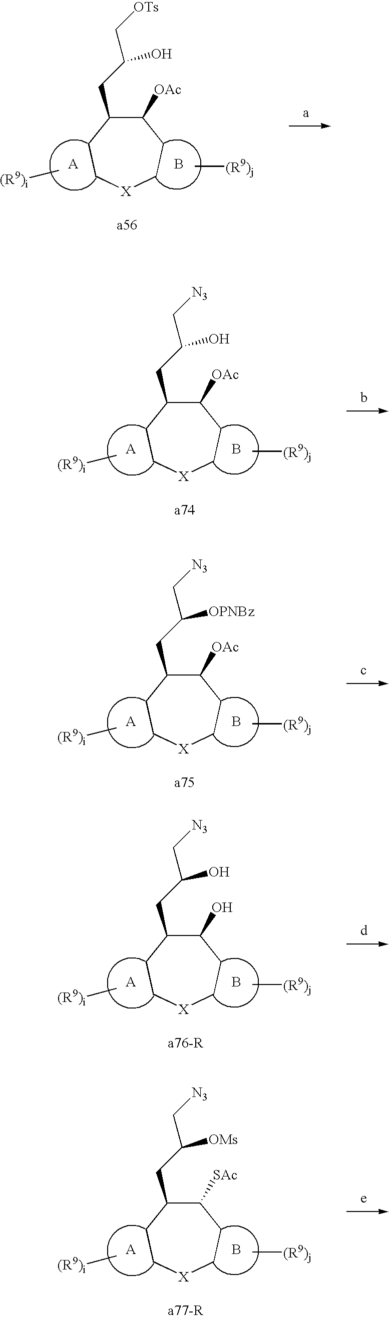

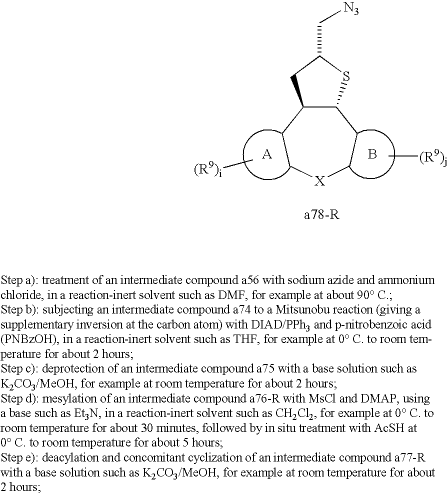

### Method L2: Synthesis of (2S,3aR,xS)-tetrahydrothiophene Intermediate Compounds

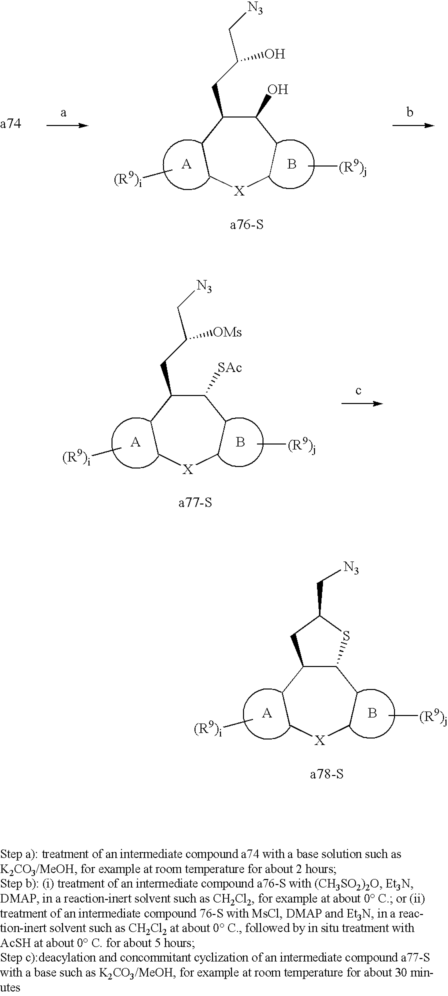

### Method L3: Synthesis of (2RS,3aR,xS)-tetrahydrothiophene Derivatives

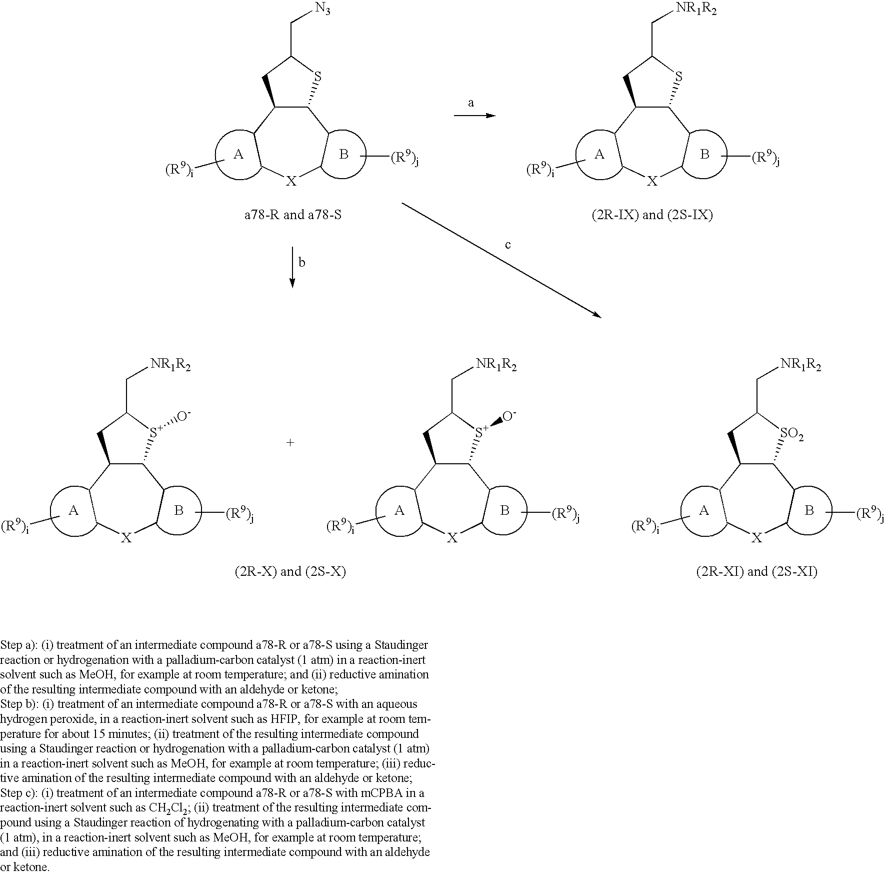

The compounds of Formula (I) may also be converted into each other following art-known transformation reactions. For instance,
a) a compound of Formula (I), wherein R¹ and R² taken together with the nitrogen atom to which they are attached form a radical of Formula (a-2), may be converted into the corresponding primary amine by treatment with hydrazine or aqueous alkali;
b) a compound of Formula (I), wherein R¹ or R² is trifluoromethylcarbonyl, may be converted into the corresponding primary or secondary amine by hydrolysis with aqueous alkali;
c) a compound of Formula (I), wherein R¹ or R² is C₁₋₆ alkyl substituted with C₁₋₆ alkylcarbonyloxy may be hydrolyzed into a compound of Formula (I) wherein R¹ or R² is C₁₋₆ alkyl substituted with hydroxy;
d) a compound of Formula (I), wherein R¹ and R² are both hydrogen may be mono- or di-N-alkylated to the corresponding amine form;
e) a compound of Formula (I), wherein R¹ and R² are both hydrogen, or R¹ or R² is hydrogen, may be N-acylated to the corresponding amide;
f) a compound of Formula (I) containing a C₁₋₆alkyloxycarbonyl group may be hydrolyzed to the corresponding carboxylic acid;
g) a compound of Formula (I) in which R⁹ is hydrogen, i.e. i and/or j is zero, can be converted to a corresponding alkyloxycarbonyl compound by treatment with an appropriate acylating agent, e.g. the appropriate alkyloxycarbonyl chloride in the presence of butyllithium in hexane using an organic solvent such as tetrahydrofuran; or
h) a compound of Formula (I) in which R⁹ is alkyloxycarbonyl can be converted to a corresponding hydroxymethyl compound by reduction for example with LiAlH₄ for example in an organic solvent such as tetrahydrofuran.

The procedures described above can be modified by the use of conventional procedures which will be known to those skilled in the art to provide analogous processes for the preparation of compounds of Formula (I).

The starting materials mentioned hereinabove are either commercially available or may be made following art-known procedures. For instance, intermediate compound 1 may be prepared in accordance with the techniques described in patent specifications W0 03/048146 and WO03/048147 referred to above or by techniques analogous thereto.

Pure stereochemically isomeric forms of the compounds of Formula (I) may be obtained by the application of art-known procedures. Diastereomers may be separated by physical methods such as selective crystallization and chromatographic techniques, e.g. counter-current distribution, liquid chromatography and the like.

The compounds of Formula (I) as prepared in the hereinabove described processes are generally racemic mixtures of enantiomers which can be separated from one another following art-known resolution procedures. The racemic compounds of Formula (I) which are sufficiently basic or acidic may be converted into the corresponding diastereomeric salt forms by reaction with a suitable chiral acid respectively with a suitable chiral base. Said diastereomeric salt forms are subsequently separated, for example, by selective or fractional crystallization and the enantiomers are liberated therefrom by alkali or acid. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound will be synthesized by stereospecific methods of preparation. These methods will advantageously employ enantiomerically pure starting materials.

The following examples are intended to illustrate and not to limit the scope of the present invention.

### Experimental Part

### A. Preparation of the Intermediate Compounds

### EXAMPLE A1

### (11R)-11-{[(4R)-2,2-dimethyl-1,3-dioxolan-4-yl]methyl}-8-fluoro-5,11-dihydro-10H-dibenzo[a,d]cyclohepten-10-one (intermediate compound 2)

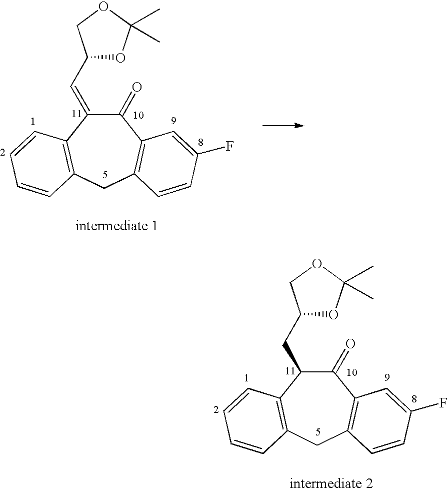

A solution of α,β-unsaturated ketone intermediate compound 1 (1.00 g, 2.96 mmol) and Et₃N (0.63 mL, 4.50 mmol) in i-PrOH (30 mL) was hydrogenated with 10% Pd/C at atmospheric pressure for 6 hours. Then the mixture was filtered through a pad of celite and the solids were washed with CH₂Cl₂ (4×20 mL). After evaporation, i-PrOH (5 mL) and Et₃N (1.20 mL) was added and the reaction mixture was stirred at 40° C. for 1 hour. The reaction mixture was cooled to room temperature and allowed to crystallize. The crystals were filtered off and dried under vacuum to afford pure intermediate compound 2 as a white crystalline powder (0.86 g, 86%); mp: 144-146° C.

Mass spectrum: CI m/z (assignment, relative intensity) 341 (MH⁺, 2%), 283 (MH⁺-acetone, 100%); EI: m/z (assignment, relative intensity) 340 (M⁺, 1%), 282 (M⁺-acetone, 79%), 226 (M⁺-sidechain+H. 100%); High resolution EI, Calculated C₂₁H₂₁FO₃ (M⁺): 340.1475, Found: 340.1479 (1%).

### EXAMPLE A2

### (10R,11R)-11-{[(4R)-2,2-dimethyl-1,3-dioxolan-4-yl]methyl}-8-fluoro-10,11-dihydro-5H-dibenzo[a,d]cyclohepten-10-ol (intermediate compound 3)

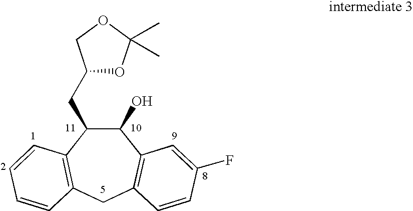

To an ice-cooled solution of intermediate compound 2 (0.42 g, 1.23 mmol) in i-PrOH (15 mL) was added phosphate buffer solution (pH=7, 5 mL) and then portionwise NaBH₄ (0.23 g, 6.16 mmol). The reaction mixture was stirred at room temperature for 1 hour. Then 10 mL NH₄Cl (sat. aq. solution) was added, the mixture was extracted with CH₂Cl₂ (3×15 mL) and the organic phases were dried with MgSO₄. After removal of the solvent, the residue was purified on a silica gel column by using ether/hexane (40:60), yielding intermediate compound 3 as a colorless oil (0.42 g, 99%).

Mass spectrum: CI m/z (assignment, relative intensity) 325 (MH⁺—H₂O, 53%), 267 (MH⁺—H₂O-acetone, 100%), 249 (MH⁺-2H₂O-acetone, 97%); EI: m/z (assignment, relative intensity) 342 (M⁺, 3%), 324 (M⁺—H₂O, 48%), 266 (M⁺—H₂O-acetone, 35%), 209 (100%); High resolution EI Calculated C₂₁H₂₃FO₃ (M⁺): 342.1631, Found: 342.1627 (5%).

### EXAMPLE A3

### (4R)-4-{[(10R,11S)-11-azido-2-fluoro-10,11-dihydro-5H-dibenzo[a,d]cyclohepten-10-yl]methyl}-2,2-dimethyl-1,3-dioxolane (intermediate compound 4)

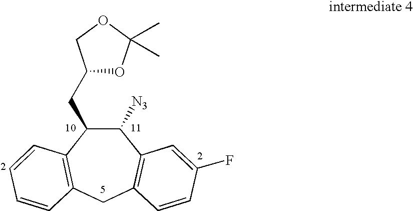

To a cooled (−30° C.) solution of DIAD (2.43 mL, 33.47 mmol) in THF (10 mL) were added intermediate compound 3 (2.30 g, 6.73 mmol) in THF (18 mL) and PPh₃ (3.71 g, 14.07 mmol). After 20 minutes, DPPA (3.62 mL, 16.83 mmol) was added and the reaction mixture was allowed to warm up to room temperature. After stirring overnight, the solvent was removed in vacuo to give a red oil. The crude material was purified by column chromatography using ether/hexane (10/90) to give an unseparated mixture of intermediate compound 4, as an oil, and Ph₃PO (3.46 g).

Mass spectrum: CI m/z (assignment, relative intensity) 368 (MH⁺, 1%), 325 (MH⁺—HN₃, 9%), 304 (13%), 276 (MH⁺—HN₃-acetone, 100%), 248 (20%).

### EXAMPLE A4

### (2R)-3-[(10R,11S)-11-azido-2-fluoro-10,11-dihydro-5H-dibenzo[a,d]cyclohepten-10-yl]-1,2-propanediol (intermediate compound 5)

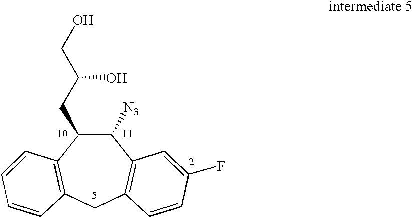

To solution of intermediate compound 4 (3.68 g, 10.02 mmol) in THF (30 mL) was added 1N HCl (30 mL) and the mixture was stirred at room temperature for 8 hours. Add K₂CO₃ (sat. aq. sol.) at 0° C., extract 3 times with CH₂Cl₂ and dry with MgSO₄. The residue obtained upon evaporation was purified by column chromatography on silica gel using Et₂O/heptane (30/70) to give an oily intermediate compound 5 (3.19 g, 91% for 2 steps from 3).

Mass spectrum: CI m/z (assignment, relative intensity) 328 (MH⁺, 2%), 310 (MH⁺—H₂O, 2%), 300 (MH⁺—N₂, 5%), 285 (MH⁺—HN₃, 11%), 267 (MH⁺—HN₃—H₂O, 100%), 249 (MH⁺—HN₃-2H₂O, 33%), 225 (MH⁺—HN₃—CH₂OHCHO, 20%).

### EXAMPLE A5

### (2R)-3-[(10R,11S)-1-azido-2-fluoro-10,11-dihydro-5H-dibenzo[a,d]cyclohepten-10-yl]-2-hydroxypropyl4-methylbenzenesulfonate (intermediate compound 6)

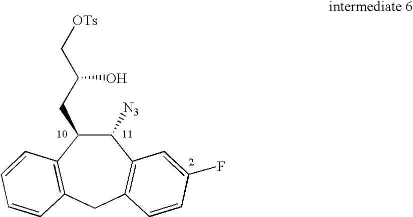

To solution of intermediate compound 5 (1.11 g, 3.39 mmol) in dry toluene (10 mL) was added Bu₂SnO (97.6 mg, 0.39 mmol), Et₃N (1.07 mL, 7.74 mmol) and TsCl (0.739 g, 3.87 mmol). The mixture was stirred at room temperature overnight. Add NH₄Cl (sat. aq. sol.), extract 3 times with CH₂Cl₂ and dry with MgSO₄. The residue was purified by column chromatography on silica gel using EtOAc/heptane (20/80) to give intermediate compound 6 as an oil (1.55 g, 95%).

Mass spectrum: -CI m/z (assignment, relative intensity) 454 (MH⁺—N₂, 1%), 421 (MH⁺—HN₃—H₂O, 1%,), 282 (MH⁺-TsOH—HN₃, 20%), 264 (MH⁺-TsOH—HN₃—H₂O, 15%), 173 (TsOH₂⁺, 100%).

### EXAMPLE A6

### (2R)-1-azido-3-[(10R,11S)-11-azido-2-fluoro-10,11-dihydro-5H-dibenzo[a,d]cyclo-hepten-10-yl]-2-propanol (intermediate compound 7)

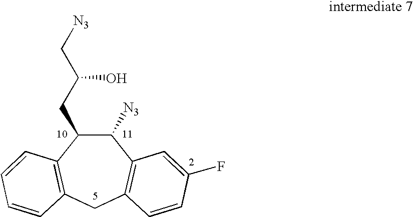

A solution of intermediate compound 6 (2.00 g, 4.15 mmol) in DMF (30 mL) was treated with sodium azide (810.8 mg, 12.47 mmol) and the mixture was stirred at 90° C. in the dark for 2 hours. The reaction mixture was diluted with water and extracted with CH₂Cl₂. The combined extracts were washed with brine. Following concentration of the dried organic phases the residue was purified by column chromatography on silica gel using heptane/EtOAc (80/20) affording diazide intermediate compound 7 (1.22 g, 88%) as an oil.

Mass spectrum: -CI m/z (assignment, relative intensity) 325 (MH⁺—N₂, 2%), 310 (MH⁺—HN₃, 3%), 297 (MH⁺—N₂—N₂, 1%), 282 (MH⁺—HN₃—N₂, 52%), 268 (MH⁺—HN₃—HN₃, 3%).

### EXAMPLE A7

### (1R)-2-azido-1-{[(10R,11S)-11-azido-2-fluoro-10,11-dihydro-5H-dibenzo[a,d]cyclo-hepten-10-yl]methyl}ethyl methanesulfonate (intermediate compound 8)

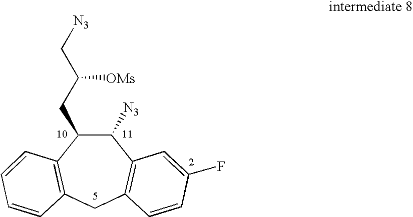

To solution of intermediate compound 7 (65 mg, 0.18 mmol) in CH₂Cl₂ (10 mL) was added DMAP (18.5 mg, 0.09 mmol), Et₃N (0.13 mL, 0.63 mmol) and MsCl (44.5 μL, 0.40 mmol). After stirring at room temperature for 10 minutes, 10 mL NH₄Cl (sat. aq. solution) was added. Extract with CH₂Cl₂ (3×10 mL) and dry with MgSO₄. Column chromatography purification on silica gel using EtOAc/heptane (20:80) as eluent yielded intermediate compound 8 as an oil (78.2 mg, 98%).

Mass spectrum: -CI m/z (assignment, relative intensity) 403 (MH⁺—N₂, 3%), 360 (MH⁺—N₂—HN₃, 43%), 307 (MH⁺—MeSO₃H—N₂, 50%), 264 (MH⁺—MeSO₃H—HN₃—N₂, 58%), 250 (MH⁺-MeSO₃H—HN₃, —N₃, 2%), 197 (100%).

### EXAMPLE A8

### [(2S,3aR,12bS)-11-fluoro-1,2,3,3a,8,12b-hexahydrodibenzo[3,4:6,7]cyclohepta[1,2-b]-pyrrol-2-yl]methanamine (intermediate compound 9)

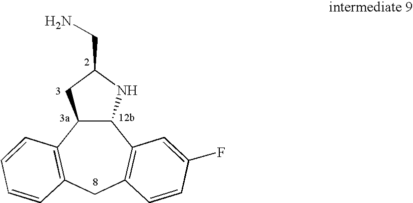

A solution of intermediate compound 8 (98.2 mg, 0.23 mmol) in MeOH (10 mL) was hydrogenated at atmospheric pressure with 10% Pd/C for 1 night. Then the mixture was filtered through a pad of celite and the solids were washed 4 times with CH₂Cl₂. After evaporation of the filtrate, the crude product was purified by column chromatography on silica gel using CHCl₃/MeOH/NH₄OH (90/9/1) as eluent. This afforded intermediate compound 9 as an oil (36.4 mg, 56%).

### EXAMPLE A9

### (1S)-2-azido-1-{[(10R,11S)-11-azido-2-fluoro-10,11-dihydro-5H-dibenzo[a,d]cyclohepten-10-yl]methyl}ethyl 4-nitrobenzoate (intermediate compound 10)

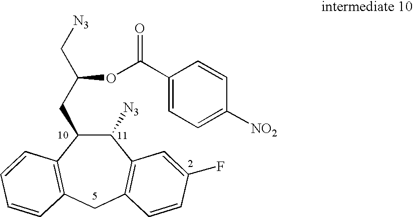

To a cooled (0° C.) solution of DIAD (4.2 mL, 21.18 mmol) in THF (50 mL) was added PPh₃ (5.55 g, 21.18 mmol). Stir at 0° C. for 30 minutes (precipitation of white solid). Then, a mixture of intermediate compound 7 (3.727 g, 10.59 mmol) and 4-nitrobenzoic acid (3.54 g, 21.18 mmol) in THF (50 mL) was added. The reaction mixture was allowed to warm up to room temperature and after stirring for 2 hours, MeOH was added and the stirring continued for an additional 30 minutes. After removal of the solvent, the crude material was purified by column chromatography on silica gel using EtOAc/heptane (20/80) to give the ester intermediate compound 10 as an oil (4.85 g, 91%).

Mass spectrum: -CI m/z (assignment, relative intensity) 431 (MH⁺—N₂—HN₃, 36%), 307 (MH⁺—N₂-p-NO₂PHCO₂H, 2%), 264 (MH⁺-p-NO₂PHCO₂H—HN₃—N₂, 58%), 197 (100%), 182 (72%).

### EXAMPLE A10

### (2s)-1-azido-3-[(10R,11S)-11-azido-2-fluoro-10,11-dihydro-5H-dibenzo[a,d]cyclo-hepten-10-yl]-2-propanol (intermediate compound 11)

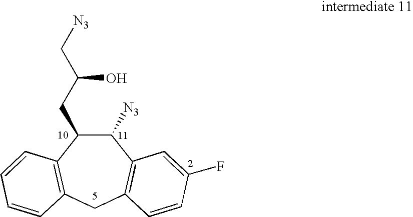

A solution of intermediate compound 10 (78.0 mg, 0.15 mmol) in MeOH (2 mL) was treated with K₂CO₃ (76.9 mg, 0.47 mmol) and the mixture was stirred for 1 hour. Add NH₄Cl (sat. aq. sol.), extract 3 times with CH₂Cl₂ and dry with MgSO₄. The residue was purified by column chromatography on silica gel using EtOAc/heptane (20/80) as eluent to give alcohol intermediate compound 11 as an oil (42.6 mg, 78%).

Mass spectrum: -CI m/z (assignment, relative intensity) 325 (MH⁺—N₂, 2%), 310 (MH⁺—HN₃, 3%), 297 (MH⁺—N₂—N₂, 1%), 282 (MH⁺—HN₃—N₂, 52%), 268 (MH⁺—HN₃—N₃, 3%).

### EXAMPLE A11

### (1S)-2-azido-1-{[(10R,11S)-11-azido-2-fluoro-10,11-dihydro-5H-dibenzo[a,d]cyclo-hepten-10-yl]methyl}ethyl methanesulfonate (intermediate compound 12)

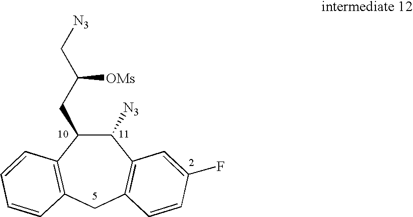

To a solution of intermediate compound 11 (42.6 mg, 0.12 mmol) in CH₂Cl₂ (5 mL) was added DMAP (12.7 mg, 0.06 mmol), Et₃N (0.047 mL, 0.42 mmol) and MsCl (33.9 μL, 0.30 mmol). Stir at room temperature for 10 minutes. Add 10 mL NH₄Cl (sat. aq. solution), extract with CH₂Cl₂ (3×10 mL) and dry with MgSO₄; upon evaporation of the solvent intermediate compound 12 was obtained as an oil (53.0 mg, 100%).

Mass spectrum: -CI m/z (assignment, relative intensity) 403 (MH⁺—N₂, 3%), 360 (MH⁺—N₂—HN₃, 43%), 307 (MH⁺-MeSO₃H—N₂, 50%), 264 (MH⁺-MeSO₃H—HN₃—N₂, 58%), 250 (MH⁺-MeSO₃H—HN₃—N₃, 21%), 197 (100%).

### EXAMPLE A12

### [(2R,3aR,12bS)-11-fluoro-1,2,3,3a,8,12b-hexahydrodibenzo[3,4:6,7]cyclohepta[1,2-b]pyrrol-2-yl]methanamine (intermediate compound 13)

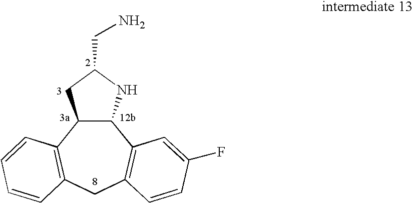

A solution of intermediate compound 12 (501.0 mg, 1.16 mmol) in MeOH (10 mL) was hydrogenated under 1 atmospheric pressure with 10% palladium-on-charcoal under vigorous stirring at room temperature for 1 night. Then the mixture was filtered through a pad of celite and the solids were washed 4 times with CH₂Cl₂. After evaporation, the crude product was purified by column chromatograhy on silica gel using CHCl₃/MeOH/NH₄OH (90/9/1). This yielded intermediate compound 13 as an oil (270.0 mg, 82%).

### EXAMPLE A13

### Benzyl [(2S,3aR,12bS)-11-fluoro-1,2,3,3a,8,12b-hexahydrodibenzo[3,4:6,7]cyclohepta[1,2-b]pyrrol-2-yl]methylcarbamate (intermediate compound 14)

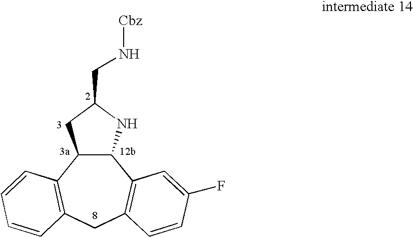

To a solution of intermediate compound 9 (220.0 mg, 0.78 mmol) in CH₂Cl₂ (5 mL) at −20° C. was added Et₃N (0.109 mL, 0.78 mmol) and benzyl chloroformate (0.112 mL, 0.78 mmol). The mixture was then stirred for 1 hour. Add 10 mL of NH₄Cl (sat. aq. solution), extract with CH₂Cl₂ (3×10 mL) and dry with MgSO₄. The residue was purified by column chromatography on silica gel using EtOAc/heptane (20/80) to give a mono-Cbz intermediate compound 14 (128.9 mg, 40%) and di-Cbz derivative (84.5 mg).

Mass spectrum. -CI m/z (assignment, relative intensity) 417 (MH⁺, 100%), 397 (MH⁺—HF, 8%), 311 (MH⁺-PhCHO, 7%), 309 (MH⁺—PHCH₂)H, 32%), 283 (16%), 252 (MH⁺-PhCH₂OCONHCH₃, 24%).

### EXAMPLE A14

### Benzyl [(2S,3aR,12bS)-1-(bromoacetyl)-11-fluoro-1,2,3,3a,8,12b-hexahydro dibenzo-[3,4:6,7]cyclohepta[1,2-b]pyrrol-2-yl]methylcarbamate (intermediate compound 15)

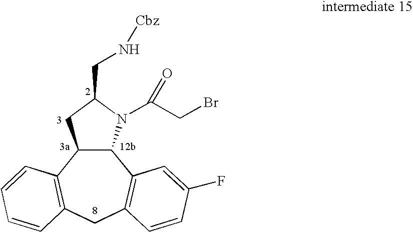

To a solution of intermediate compound 14 (32.5 mg, 0.078 mmol) in EtOAc (3 mL) was added 1 mL of NaOH (sat. aq. solution) and bromoacetyl bromide (6.8 μL, 0.078 mmol). The two phases were stirred vigorously for 1 night. Add 10 mL of NH₄Cl (sat. aq. solution), extract with CH₂Cl₂ (3×10 mL) and dry with MgSO₄. Column chromatography purification on silica gel using EtOAc/heptane (20/80) gave intermediate compound 15 as an oil (31.4 mg, 62%).

Mass spectrum: -CI m/z (assignment, relative intensity) 457 (MH⁺—HBr, 3%,), 413 (MH⁺—HBr—CO₂, 1%), 365 (MH⁺—HBr-PhCH₃ 1%), 351 (MH⁺-PhCHO—HBr, 2%), 323 (MH⁺—HBr-PhCHO—CO, 5%), 119 (8%), 91 (100%).

### EXAMPLE A15

### Benzyl (5 aS,14bR,15aS)-7-fluoro-4-oxo-1,3,4,5a,10,14b,15,15a-octahydro-2H-dibenzo[3′,4′:6′,7′]cyclohepta[1′,2′:4,5]pyrrolo[1,2-a]pyrazine-2-carboxylate (intermediate compound 16)

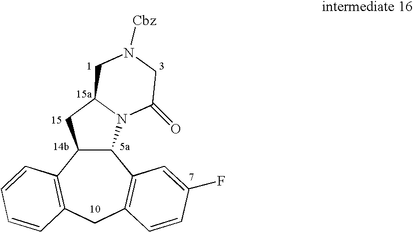

To a solution of intermediate compound 15 (91.7 mg, 0.17 mmol) in DMF (5 mL) was added K₂CO₃ (103.0 mg, 0.75 mmol) and the mixture was stirred at room temperature for 36 hours. Add 10 mL of NH₄Cl (sat. aq. solution), extract with CH₂Cl₂ (3×10 mL) and dry with MgSO₄. Column chromatography purification on silica gel using EtOAc/heptane (30/70) gave polycyclic intermediate compound 16 (86.2 mg, 92%) as an oil.

Mass spectrum: -CI m/z (assignment, relative intensity) 457 (MH⁺, 1%), 323 (MH⁺-PhCHO—CO, 5%), 279 (MH⁺-Cbz-CH₂CO, 1%), 91 (10%).

### EXAMPLE A16

### N-{[(2R,3aR,12bS)-11-fluoro-1,2,3,3a,8,12b-hexahydrodibenzo[3,4:6,7] cyclohepta-[1,2-b]pyrrol-2-yl]methyl}(triphenyl)methanamine (intermediate compound 17)

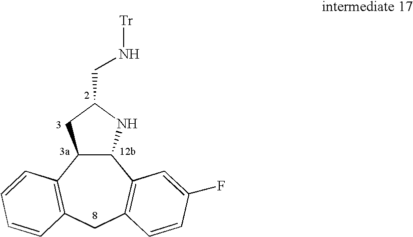

To an ice cooled solution of intermediate 13 (41.6 mg, 0.15 mmol) in CH₂Cl₂ (5 mL) was added Et₃N (42.5 μL, 0.3 mmol), DMAP (9.4 mg, 0.07 mmol) and TrCl (46.1 mg, 0.16 mmol). The mixture was then stirred at 0° C. for 2 hours. Add 10 mL of NH₄Cl (sat. aq. solution), extract with CH₂Cl₂ (3×10 mL) and dry with MgSO₄. Column chromatography purification on silica gel using EtOAc/heptane (20/80) gave a crystalline intermediate compound 17 (52.6 mg, 68%); mp: 58-60° C.

Mass spectrum: -APCI m/z (assignment, relative intensity) 525 (MH⁺, 38%), 390 (4%), 283 (MH⁺-(Tr-H), 15%), 252 (MH⁺—CH₃NHTr, 27%), 243 (Tr⁺, 100%), 228 (7%), 165 (29%).

### EXAMPLE A17

### N-{[(2R,3aR,12bS)-1-(bromoacetyl)-11-fluoro-1,2,3,3a,8,12b-hexahydrodibenzo-[3,4:6,7]cyclohepta[1,2-b]pyrrol-2-yl]methyl}(triphenyl)methanamine (intermediate compound 18)

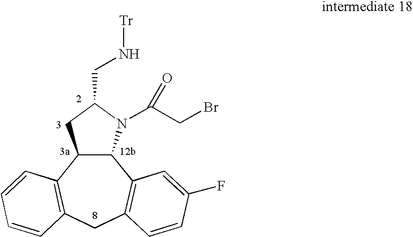

The intermediate compound 17 (26.7 mg, 0.05 mmol) was added to a two-phase system consisting of 2 mL CH₂Cl₂ and 0.5 mL Na₂CO₃ (aq. sat. solution), and the mixture was stirred for 10 minutes. After adding bromoacetyl bromide (6.8 μL, 0.08 mmol) the two phases were stirred vigorously for 3 hours. Extract with CH₂Cl₂ (3×10 mL) and dry with MgSO₄. Column chromatography purification on silica gel using EtOAc/heptane (20/80) gave intermediate compound 18 as an oil (27.9 mg, 85%) characterised as a mixture of two conformers.

Mass spectrum: -APCI m/z (assignment, relative intensity) 645 (MH⁺, 39%), 601 (3%), 403 (MH⁺-(Tr-H), 7%), 321 (MH⁺-TrH—HBr, 21%), 243 (Tr⁺, 100%), 228 (3%), 165 (15%).

### EXAMPLE A18

### N-{[(2R,3 aR,12bS)-11-fluoro-1-(methoxyacetyl)-1,2,3,3a,8,12b-hexahydrodibenzo-[3,4:6,7]cyclohepta[1,2-b]pyrrol-2-yl]methyl}(triphenyl)methanamine (intermediate compound 19)

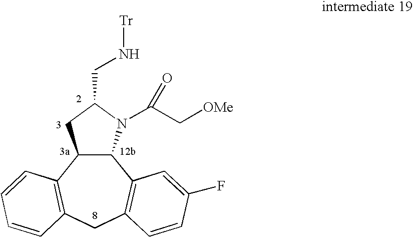

To a solution of intermediate compound 18 (530 mg, 0.82 mmol) in MeOH (15 mL) was added MeSO₃H (3 mL) and the mixture was stirred at 60° C. for 30 minutes. After complete evaporation of the solvent, the residue was dissolved in CH₂Cl₂/K₂CO₃ (sat. aq. solution) (15/15 mL) and the organic layer was separated. The aqueous layer was extracted with CH₂Cl₂ (3×10 mL) and the combined organic layers were then dried with MgSO₄. Column purification on silica gel using EtOAc/heptane (20/80) gave intermediate compound 19 as an oil (231.3 mg, 47%), characterised as a mixture of two conformers.

Mass spectrum: -APCI m/z (assignment, relative intensity) 598 (MH⁺, 1%), 519 (2%), 355 (MH⁺-Tr, 13%), 283 (MH⁺-Tr-CO═CHOMe, 2%), 271 (10%), 243 (Tr⁺, 100%), 167 (21%).

### EXAMPLE A19

### [(2R,3aR,12bS)-1-(bromoacetyl)-11-fluoro-1,2,3,3a,8,12b-hexahydrodibenzo [3,4:6,7]-cyclohepta[1,2-b]pyrrol-2-yl]methylformamide (intermediate compound 20)

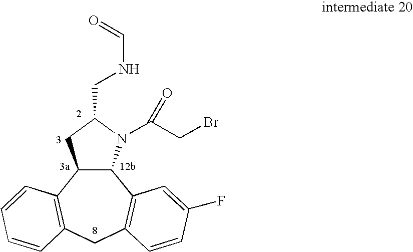

The intermediate compound 18 (100 mg, 0.15 mmol) was dissolved in 98% formic acid (2 mL) and the mixture was stirred at room temperature for 24 hours. After removal of excess of formic acid in vacuo, the residue was dissolved in CHCl₃ (2 mL) and EEDQ (47 mg, 0.19 mmol) was added. The solution was stirred at room temperature for 5 hours. Following evaporation of the solvent, the residue was purified by column chromatography on silica gel using CH₂Cl₂/MeOH (98/2) as eluent. The intermediate compound 20 (54.7 mg, 82%) was obtained as an oil.

Mass spectrum: -CI m/z (assignment, relative intensity) 431, 433 (MH^{+, 42}%), 353 (MH⁺—HBr, 100%), 294 (MH⁺—HBr—CH₃NHCHO, 9%), 249 (4%), 158 (2%), 130 (7%).

### EXAMPLE A20

### (5aS,14bR,15 aR)-7-fluoro-4-oxo-1,3,4,5a,10,14b,15,15a-octahydro-2H-dibenzo-[3′,4′:6′,7′]cyclohepta[1′,2′:4,5]pyrrolo[1,2-a]pyrazine-2-carbaldehyde (intermediate compound 21)

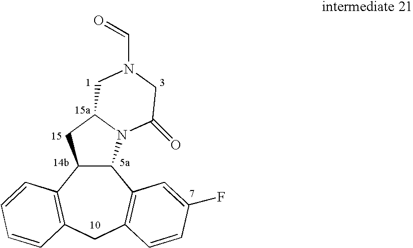

To a solution of intermediate compound 20 (91 mg, 0.21 mmol) in dry THF (10 mL) was added a solution of t-BuOK (30.3 mg, 0.24 mmol) in THF (2 mL). The reaction mixture was stirred at room temperature for 30 minutes. Water (10 mL) was then added and the mixture extracted with CH₂Cl₂ (10 mL). Column chromatography purification on silica gel using CH₂Cl₂/MeOH (97/3) gave the cyclic intermediate compound 21 (47.4 mg, 64%) as an oil.

Mass spectrum: -CI m/z (assignment, relative intensity) 351 (MH⁺, 100%), 331 (MH⁺—HF, 5%), 323 (MH⁺—CO, 6%), 319 (8%), 219 (2%), 130 (4%).

### EXAMPLE A21

### (10R,11R)-11-{[(4R)-2,2-dimethyl-1,3-dioxolan-4-yl]methyl}-8-fluoro-10,11-dihydro-5H-dibenzo[a,d]cyclohepten-10-yl acetate (intermediate compound 22)

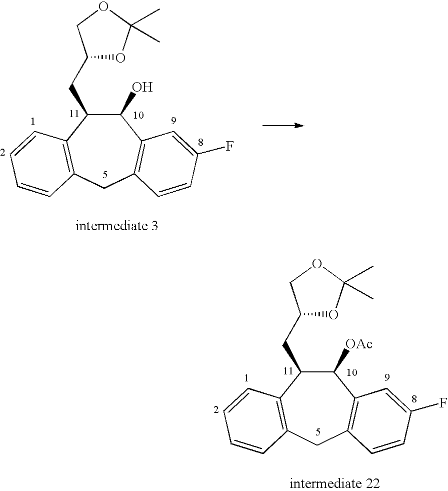

To a solution of intermediate compound 3 (0.42 g, 1.23 mmol) in CH₂Cl₂ (30 mL) was added Et₃N (0.43 mL, 3.07 mmol), DMAP (0.15 g, 1.23 mmol) and AcOH anhydride (0.29 mL, 3.07 mmol). Stir at room temperature for 1 hour, add NH₄Cl (sat. aq. solution, 20 mL), extract with CH₂Cl₂ (3×15 mL) and dry with MgSO₄. Column chromatography purification on silica gel using ether/hexane (30:70) gave a white crystalline intermediate compound 22 (0.45 g, 95%); mp:147-149° C.

Mass spectrum: -CI m/z (assignment, relative intensity) 385 (MH⁺, 1%), 325 (MH⁺-AcOH, 100%), 267 (MH⁺-AcOH-acetone, 43%), 249 (MH⁺-AcOH-acetone-H₂O, 47%);

EI: m/z (assignment, relative intensity) 324 (M⁺-AcOH, 46%), 266 (M⁺-AcOH-acetone, 20%), 209 (M⁺-AcOH-sidechain, 100%); High resolution EI Calculated C₂₂H₂₁FO₂ (M⁺-AcOH): 324.1526, Found: 324.1521 (M⁺, 72%).

### EXAMPLE A22

### (10R,11R)-11-[(2R)-2,3-dihydroxypropyl]-8-fluoro-10,11-dihydro-5H-dibenzo[a,d]-cyclohepten-10-yl acetate (intermediate compound 23)

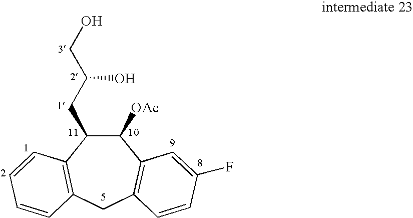

To a solution of intermediate compound 22 (0.45 g, 1.17 mmol) in THF (10 mL) was added 1N HCl (10 mL). After stirring at room temperature for 8 hours, 10 mL Na₂CO₃ (sat. aq. solution) was added at 0° C. Extract with CH₂Cl₂ (3×10 mL) and dry with MgSO₄. Column chromatography purification on silica gel using EtOAc/hexane (70:30) gave diol intermediate compound 23 as a colorless oil (0.39 g, 96%).

Mass spectrum: -CI m/z (assignment, relative intensity) 345 (MH⁺, 1%), 327 (MH⁺—H₂O, 3%), 309 (MH⁺-2 H₂O, 3%), 285 (MH⁺-AcOH, 17%), 267 (MH⁺-AcOH-H₂O, 100%), 249 (M⁺-AcOH-2 H₂O, 3%); EI: m/z (assignment, relative intensity) 326 (M⁺—H₂O, 10%), 284 (M⁺-AcOH, 13%), 209 (M⁺-AcOH-sidechain, 100% )); High resolution EI Calculated C₂₀H₁₉FO₃ (M⁺—H₂O): 326.1318, Found: 326.1316 (31%).

### EXAMPLE A23

### (10R,11R)-11-[(2S)-2,3-diazidopropyl]-8-fluoro-10,11-dihydro-5H-dibenzo[a,d]-cyclohepten-10-yl acetate (intermediate compound 24)

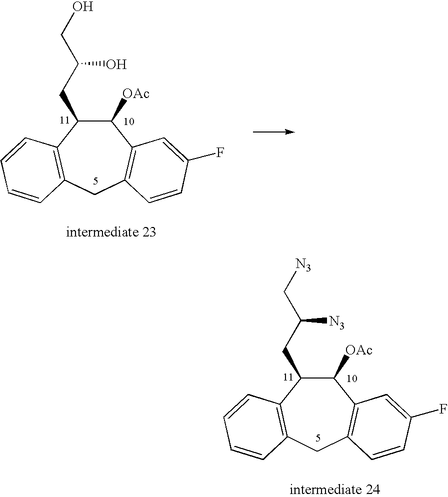

To the intermediate compound 23 (0.59 g, 1.72 mmol) in CH₂Cl₂ (15 mL) was added Et₃N (0.96 mL, 6.86 mmol), DMAP (209 mg, 1.72 mmol) and MsCl (0.53 mL, 6.86 mmol) at 0° C. Stir at room temperature for 1 hour. Work it up by adding NH₄Cl (sat. aq. sol.), extract 3 times with CH₂Cl₂ and dry with MgSO₄. Column purification on silica gel using EtOAc/heptane (50/50) afforded dimesyl compound as an oil (0.84 g, 98%). To this intermediate compound (182.5 mg, 0.36 mmol) in DMF (10 mL) was added NaN₃ (95 mg, 1.46 mmol). The reaction mixture was heated at 80° C. for 3 hours. After cooling, add NH₄Cl (sat. aq. sol.), extract 3 times with CH₂Cl₂ and dry with MgSO₄. After evaporation the residue was purified on silica gel using EtOAc/heptane (20/80) to give intermediate compound 24 as an oily product (122.3 mg, 85%).

### EXAMPLE A24

### (4S)-4-{[(10R,11R)-2-fluoro-11-hydroxy-10,11-dihydro-5H-dibenzo[a,d]cyclohepten-10-yl]methyl}-2-imidazolidinone (intermediate compound 25)

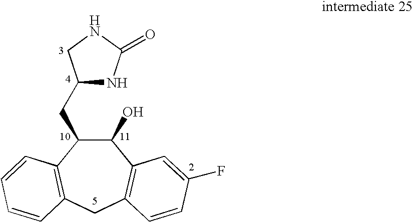

Intermediate compound 24 was converted via diazido alcohol intermediate compound 24a into a diamine which was further converted into intermediate compound 25. To a solution of intermediate compound 24 (120.1 mg, 0.30 mmol) in MeOH (10 mL) was added K₂CO₃ (126.4 mg, 0.91 mmol). The reaction mixture was stirred at room temperature for 1 hour. Add NH₄Cl (sat. aq. sol.), extract 3 times with CH₂Cl₂. Column purification on silica gel using Et₂O/heptane (40/60) gave the diazido alcohol intermediate compound 24a as an oily product (77.5 mg, 72%). This compound (75 mg, 0.21 mmol) in MeOH (5 mL) was hydrogenated at 1 atmospheric pressure with 10% palladium-on-charcoal under vigorous stirring at room temperature for 1 night. Then the mixture was filtered through a pad of celite and the solids were washed 4 times with CH₂Cl₂. After evaporation of the solvent, the crude product was dissolved in 5 mL of CH₃CN and Et₃N (34 μL, 0.24 mmol) was added. The reaction mixture was heated under argon at 70° C. After 1 hour, a solution of diphenyl carbonate (23 mg, 0.11 mmol) in CH₃CN was added dropwise and the mixture was stirred at 70° C. for 1 day. After evaporation, the crude product was purified by column chromatography on silica gel using CHCl₃/MeOH (90/10) to give the imidazolidinone intermediate compound 25 as an oil (34.4 mg, 48%).

### EXAMPLE A25

### (11E)-1-{1[(4R)-2,2-dimethyl-1,3-dioxolan-4-yl]methylene}-8-fluorodibenzo[b,f]-oxepin-10(11H)-one (intermediate compound 27)

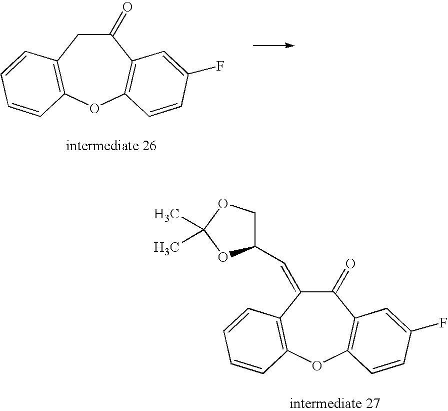

To a suspension of intermediate compound 26 (0.228g, 1 mmol) and MgBr₂ (0.202 g, 1.1 mmol) in dry toluene (5 mL), (S)-glyceraldehyde acetonide (4 mmol, 1.5 M solution in THF) and t-BuOK (22.4 mg, 0.2 mmol) was added and stirred for 3 hours at room temperature. A saturated aq. NH₄Cl solution (5 mL) was added, the organic layer was separated and kept over anhydrous MgSO₄. The solvent was removed under reduced pressure followed by the separation of α,β-unsaturated product by flash column chromatography using EtOAc:heptane (1:9)eluent to obtain intermediate compound 27 as a yellow liquid in a ratio of 85/15 E and Z isomer (85%, 0.289 g).

HRMS: Calculated 340.1111; found 340.1122

### EXAMPLE A26

### a)(10R,11R)-11-{[(4R)-2,2-dimethyl-1,3-dioxolan-4-yl]methyl}-8-fluoro-10,11-dihydrodibenzo[b,f]oxepin-10-ol (intermediate compound 29)

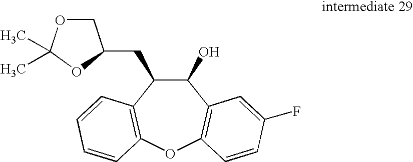

To a solution of intermediate compound 27 (0.340 g, 1 mmol) in i-PrOH (5 mL) was added Et₃N (0.21 mL, 1.5 mmol) and the reaction mixture was hydrogenated under atmospheric pressure using 10% Pd/C (40 mg) as a catalyst. After completion of the reaction (4 hours) the reaction mixture was passed through a small pad of celite and further washed with CH₂Cl₂ (2×5 mL) followed by the evaporation of the solvent to obtain crude ketone intermediate compound 28.

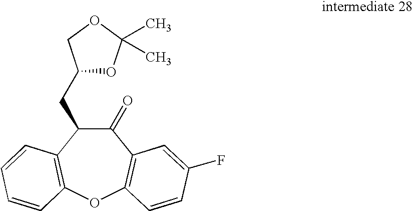

b) The crude intermediate compound 28 thus obtained was dissolved in i-PrOH (10 mL) and aqeous phosphate buffer solution (3 mL, pH 7) was added to it. The temperature was lowered to 0° C. and NaBH₄ (0.152 g, 4 mmol) was added to it in several lots and then allowed to stir further for 15 minutes at the same temperature. Aq. NH₄Cl solution (5 mL) was added and the reaction mixture was extracted using Et₂O (3×5 mL). After drying over anhydrous MgSO₄ the solvent was removed under reduced pressure and the two diastereomeric alcohols (1:1) with slightly different polarity were separated by flash column chromatography using EtOAc:heptane (20:80) as an eluent to obtain the more polar cis-alcohol intermediate compound 29 as a white solid (mp: 59-61° C.; 49%, 0.16 g).

HRMS: Calculated 344.1424; found 344.1435

### EXAMPLE A27

### (10S,11R)-11-{[(4R)-2,2-dimethyl-1,3-dioxolan-4-yl]methyl}-8-fluoro-10,11-dihydrodibenzo[b]floxepin-10-yl azide (intermediate compound 30)

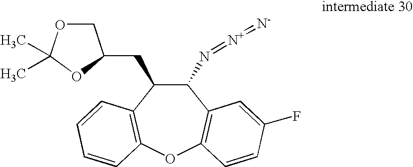

To a solution of PPh₃ (0.524 g, 2 mmol) in dry THF (5 mL) at −15° C., a solution of DIAD (0.424 g, 2.1 mmol) in THF (2 mL) was added and the resulting complex was stirred for 20 minutes followed by the addition of intermediate compound 29 (0.329 g, 1 mmol) dissolved in THF (2 mL) and a solution of DPPA (0.330 g, 1.2 mmol) in THF (1 mL). The reaction mixture was warmed to room temperature and stirred for 18 hours. After addition of MeOH the reaction mixture was dried under vaccum followed by separation of the azide using flash columnchromatography using EtOAc:heptane (1:9) as an eluent to obtain intermediate compound 30 as a colourless liquid (91%, 0.335 g).

HRMS: Calculated 369.1489; found 369.1483.

### EXAMPLE A28

### (2R)-3-[(10R,11S)-1-azido-2-fluoro-10,11-dihydrodibenzo[b,f]oxepin-10-yl]-1,2-propanediol (intermediate compound 31)

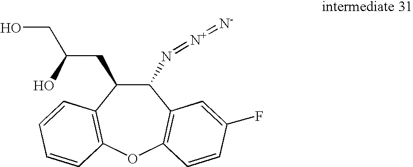

To a solution of intermediate compound 30 (0.369 g, 1 mmol) in THF (5 mL) 1M aq. HCl solution (1 mL) was added and stirred for 18 hours. THF was removed under reduced pressure and the diol was extracted using Et₂O (3×10 mL). The organic layer was treated with aq. NaHCO₃ (5 mL) followed by a brine wash (5 mL). After drying over anhydrous MgSO₄ the solvent was removed under vacuum to obtain intermediate compound 31 as a thick viscous liquid (95%, 0.313 g).

HRMS: Calculated 329.1176; found 329.1184.

### EXAMPLE A29

### (2R)-1-[(10R,11S)-11-azido-2-fluoro-10,11-dihydrodibenzo[b,f]oxepin-10-yl]-3-(trityloxy)-2-propanol (intermediate compound 32)

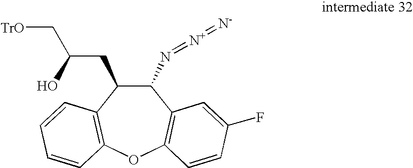

To a solution of intermediate compound 31 (0.329 g, 1 mmol) in CH₂Cl₂ (10 mL) Et₃N (0.28 mL, 2 mmol), DMAP (0.1 mmol, 12.2 mg) and TrCl (0.307g, 1.1 mmol) were added and stirred for 24 hours. The solvent was removed under reduced pressure and the crude reaction mixture was subjected to flash column chromatography using EtOAc:heptane (1:9) as an eluent to obtain intermediate compound 32 as a white solid (mp: 58-59° C.; 80%, 0.456 g).

HRMS: Calculated 571.2271; found 571.2286.

### EXAMPLE A30

### (1R)-2-[(10R, 11S)-11-azido-2-fluoro-10,11-dihydrodibenzo[b,f]oxepin-10-yl]-1-[(trityloxy)methyl]ethyl methanesulfonate (intermediate compound 33)

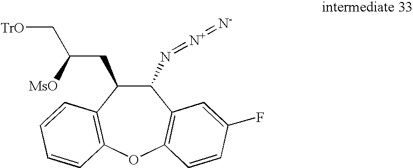

To a solution of intermediate compound 32 (0.571 g, 1 mmol) in CH₂Cl₂ at −10° C., Et₃N (0.28 mL, 2 mmol), DMAP (12.2 mg, 0.1 mmol) and MsCl (0.126 g, 1.1 mmol) were added. The reaction mixture was warmed up to room temperature and stirred for 4 hours. Water (3 mL) was added and the organic layer was separated and dried over anhydrous MgSO₄ followed by the purification by flash chromatography using EtOAc:heptane (1:9) as an eluent to obtain intermediate compound 33 as a white solid (mp:55-56° C.; 85%, 0.515 g).

HRMS: Calculated 649.2047; found 649.2064

### EXAMPLE A31

### (1R)-2-[(10R,11S)-11-azido-2-fluoro-10, l-dihydrodibenzo[b,f]oxepin-10-yl]-1-(hydroxymethyl)ethyl methanesulfonate (intermediate compound 34)

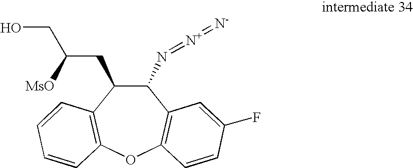

To a solution of intermediate compound 33 (0.649 g, 1 mmol) in MeOH (5 mL) amberlyst-15 (0.1 g) was added and the reaction mixture was stirred at 40° C. for 3 hours, then filtered to remove the catalyst. The solvent was removed under reduced pressure and the product purified by flash column chromatography using EtOAc:heptane (2:8) as an eluent to obtain intermediate compound 34 as a thick viscous liquid (90%, 0.366 g).

HRMS: Calculated 407. 0951; found 407.0975.

### EXAMPLE A32

### (10R,11S)-11-azido-2-fluoro-10-[(2S)-oxiranylmethyl]-10,11-dihydrodibenzo[b,f]-oxepine (intermediate compound 35)

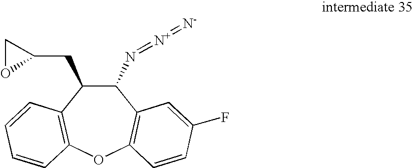

A mixture of intermediate compound 34 (0.407 g, 1 mmol) and K₂CO₃ (0.276 g, 2 mmol) was stirred in i-PrOH (10 mL) for 8 hours, filtered to remove K₂CO₃ and the solvent was removed under reduced pressure. The product was purified by flash chromatograpy using EtOAc: heptane (2:8) as an eluent to obtain intermediate compound 35 as a colourless liquid (78%, 0.242 g).

HRMS: Calculated 311.10 70; found 311.1089.

### EXAMPLE A33

### [(2R,3aR,12bS)-11-fluoro-2,3,3a,12b-tetrahydro-1H-dibenzo[2,3:6,7]oxepino[4,5-b]-pyrrol-2-yl]methanol (intermediate compound 36)

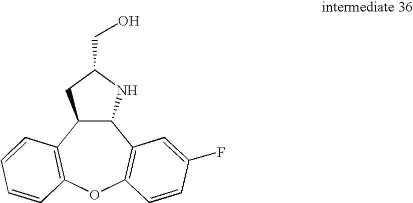

To a solution of intermediate compound 35 (0.311 g, 1 mmol) in i-PrOH (10 mL), Et₃N (0.140 mL, 1 mmol) was added. The mixture was hydrogenated under atmospheric pressure using 10% Pd/C (50 mg) as a catalyst. After completion of the reaction (3 hours), it was passed through a small pad of celite and the catalyst was washed with CH₂Cl₂ (2×5 mL). The combined organic layers were evaporated under reduced pressure and purified by flash column chromatography using EtOAC:heptane (1:1) as an eluent to obtain intermediate compound 36 as a white solid (mp: 108-109° C.; 83%, 0.236 g).

HRMS: Calculated 285.1165; found 285.1172.

### EXAMPLE A34

### Methyl (2R,3aR ,12bS)-11-fluoro-2-(hydroxymethyl)-2,3,3a,12b-tetrahydro-1H-di-benzo[2,3:6,7]oxepino[4,5-b]pyrrole-1-carboxylate (intermediate compound 37).

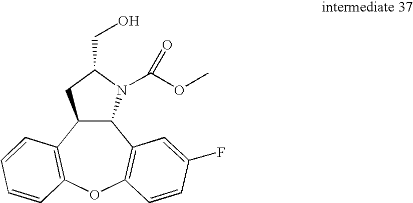

To a solution of intermediate compound 36 (0.14 g, 0.5 mmol) in CH₂Cl₂ (4 mL) at 0° C. a saturated solution (aq.) of NaHCO₃ (2 mL) was added. After the addition of methylchloroformate (1.5 eq.), the reaction mixture was stirred vigorously at 0° C. for 20 minutes, warmed up to room temperature and allowed to stir further for 0.5 hour. The organic layer was separated, dried over MgSO₄ and purified by flash column chromatography using EtOAc:heptane (4:6) as an eluent to obtain intermediate compound 37 as a thick viscous liquid (83%, 0.14 g).

HRMS: Calculated 343.1220; found 343.1218.

### EXAMPLE A35

### Methyl (2R,3aR,12bS)-2-(aminomethyl)-11-fluoro-2,3,3a,12b-tetrahydro-1H-dibenzo-[2,3:6,7]oxepino[4,5-b]pyrrole-1-carboxylate (intermediate compound 38)

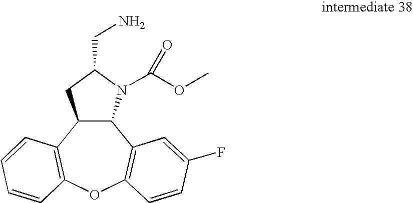

To a solution of PPh₃ (0.26 g, 1 mmol) in dry THF (4 mL) at −15° C. a solution of DIAD (0.22 g, 1.1 mmol) in THF (1 mL) was added and the resulting complex was stirred for 20 minutes. After the addition of intermediate compound 37 (0.17 g, 0.5 mmol) dissolved in THF (1 mL) and DPPA (0.14 g, 0.5 mmol) in THF (1 mL), the reaction was warmed to up room temperature and stirred for 18 hours. An excess of PPh₃ (5 eq) and water (0.5 mL) was added to the reaction mixture and then heated at 40° C. for 3 hours to reduce the azide to amine functionality. Silica gel was added to the reaction mixture and the solvent was removed under reduced pressure followed by purification of the product by flash column chromatography using CH₂Cl₂:MeOH (9:1) as an eluent to obtain intermediate compound 38 as a thick viscous liquid (80%, 0.14 g).

HRMS: Calculated 342.1380; found 342.1376.

### EXAMPLE A36

### {(2R,3aR,12bS)-11-fluoro-1-[(2-nitrophenyl)sulfonyl]-2,3,3a,12b-tetrahydro-1H-dibenzo[2,3:6,7]oxepino[4,5-b]pyrrol-2-yl}methyl 2-nitrobenzenesulfonate (intermediate compound 39)

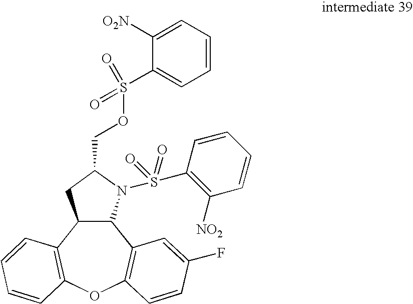

To a solution of intermediate compound 36 (0.5 mmol, 0.14g), Et₃N (5 eq.) and DMAP (20 mol %) in CH₂Cl₂ at −20° C., NsCl (3 eq.) was added. Reaction mixture was warmed up to room temperature and left for overnight stirring. Aqueous NaHCO₃ (2 mL) was added to the reaction mixture and the organic layer was separated and dried over MgSO₄. Following column chromatography (SiO₂) using EtOAc:heptane (1:1) as an eluent yielded intermediate compound 39 as a yellow crystalline solid (mp: 88-90° C., 71%, 0.23 g).

### EXAMPLE A37

### a) (10R,11R)-8-fluoro-11-((2R)-2-hydroxy-3-{[(4-methylphenyl)sulfonyl]oxy}propyl)-10,11-dihydro-5H-dibenzo[a,d]cyclohepten-10-yl acetate (intermediate compound 23a)

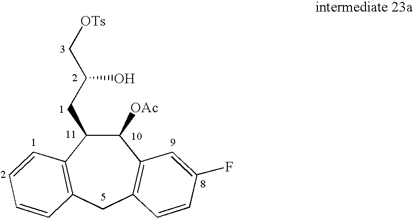

To a solution of intermediate compound 23 (0.12 g, 0.355 mmol) in dry toluene (10 mL) was added n-Bu₂SnO (9 mg, 0.036 mmol), Et₃N (0.13 mL, 0.888 mmol) and TsCl (0.10 g, 0.533 mmol). Stir at room temperature for 24 hours, add NH₄Cl (sat. aq. solution, 10 mL), extract with CH₂Cl₂ (3×10 mL) and dry with MgSO₄. Column chromatography purification on silica gel using EtOAc/hexane (30:70) yielded intermediate compound 23a as a colorless oil (0.15 g, 84%).

Mass spectrum: CI m/z (assignment, relative intensity) 481 (MH⁺—H₂O, 1%), 439 (MH⁺-AcOH, 4%), 421 (MH⁺-AcOH-H₂O, 1%), 267 (MH⁺-AcOH-TSOH, 18%), 249 (MH⁺-AcOH-TsOH—H₂O, 100%); EI: m/z (assignment, relative intensity) 480 (M⁺—H₂O, 1%), 438 (M⁺-AcOH, 36%), 266 (M⁺-AcOH-TsOH, 15%), 248 (^{M+}-AcOH-TsOH—H₂O, 18%); High resolution EI Calculated C₂₅H₂₃FO₄S (M⁺-AcOH): 438.1301, Found: 438.1300 (51%).

### b) (10R,11R)-11-[(2R)-3-azido-2-hydroxypropyl]-8-fluoro-10,11-dihydro-5H-dibenzo-[a,d]cyclohepten-10-yl acetate (intermediate compound 40)

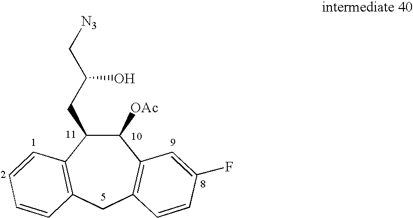

To a solution of intermediate compound 23a (1.30 g, 2.61 mmol) in DMF (25 mL) was added NaN₃ (0.51 g, 7.83 mmol). The reaction mixture was heated at 100° C. for 1 night. After cooling, add NH₄Cl (sat. aq. sol.), extract 3 times with CH₂Cl₂ and dry with MgSO₄. After evaporation the residue was purified by column chromatography on silica gel using EtOAc/heptane (20/80) to give intermediate compound 40 as an oily product (0.79 g, 82%).

### EXAMPLE A38

### (10R,11R)-11-[(2R)-3-azido-2-hydroxypropyl]-8-fluoro-10,11-dihydro-5H-dibenzo-[a,d]cyclohepten-10-ol (intermediate compound 41)

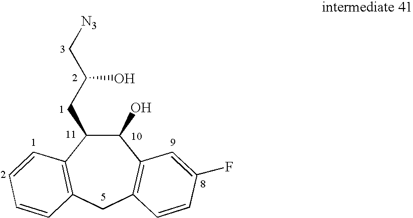

A solution of intermediate compound 40 (454.9 mg, 1.23 mmol) in MeOH (10 mL) was treated with K₂CO₃ (340.1 mg, 2.46 mmol) and the mixture was stirred at room temperature for 1 hour. Add NH₄Cl (sat. aq. sol.), extract 3 times with CH₂Cl₂ and dry with MgSO₄. The solution was filtered and evaporated and the residue was purified by column chromatography on silica gel using EtOAc/heptane (30/70) to give diol intermediate compound 41 (370.9 mg, 92%).

### EXAMPLE A39

### S-((10S,11R)-11-{(2R)-3-azido-2-[(methylsulfonyl)oxy]propyl}-8-fluoro-10,11-dihydro-5H-dibenzo[a,d]cyclohepten-10-yl)ethanethioate (intermediate compound 42)

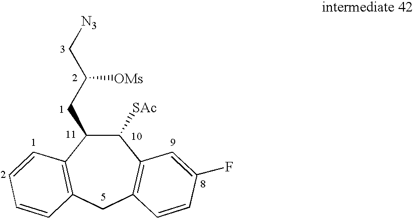

To intermediate compound 41 (670 mg, 2.05 mmol) in CH₂Cl₂ (25 mL) was added Et₃N (2.30 mL, 16.4 mmol), DMAP (0.13 mg, 1.02 mmol) and (CH₃SO₂)₂O (1.07 g, 6.15 mmol) at 0° C. Stir at room temperature for 1 hour, cool to 0° C. again, add AcSH (0.44 ml, 6.15 mmol) and stir at room temperature for 4 hours. Work up by adding NH₄Cl (sat. aq. sol.). Extract 3 times with CH₂Cl₂. Column chromatography on silica gel using CH₂Cl₂ (100%) afforded intermediate compound 42 as an oil (0.68 g, 72%).

### EXAMPLE A40

### (2S,3aR,12bS)-2-(azidomethyl)-11-fluoro-3,3a,8,12b-tetrahydro-2H-dibenzo [3,4:6,7]-cyclohepta[1,2-b]thiophene (intermediate compound 43)

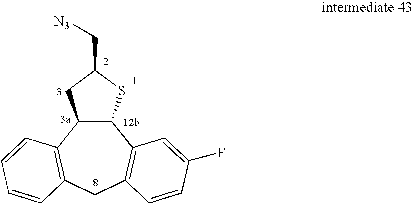

To intermediate compound 42 (0.15 g, 0.33 mmol) in MeOH (5 mL) was added K₂CO₃ (92 mg, 0.67 mmol). After stirring at room temperature for 1 night, the mixture was worked up by adding NH₄Cl (sat. aq. sol.). Extract 3 times with CH₂Cl₂ and dry with MgSO₄. Column chromatography purification on silica gel using CH₂Cl₂/heptane (40/60) gave intermediate compound 43 as an oily product (76 mg, 70%).

Mass spectrum: CI m/z (assignment, relative intensity) 326 (MH⁺, 25%), 298 (MH⁺—N₂, 60%), 283 (MH⁺—HN₃, 100%), 269 (MH⁺—N₂—CH₂NH, 12%), 249 (MH⁺—HN₃—H₂S, 25%), 235 (MH⁺—N₂—CH₂NH—H₂S, 21%), 197 (61%).

### EXAMPLE A41

### (2S,3aR,12bS)-2-(azidomethyl)-11-fluoro-3,3a,8,12b-tetrahydro-2H-dibenzo [3,4:6,7]-cyclohepta[1,2-b]thiophene 1,1-dioxide (intermediate compound 44)

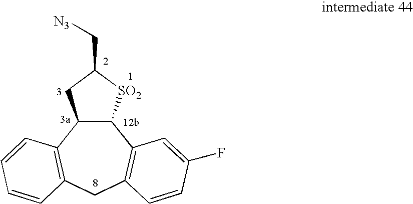

To a solution of intermediate compound 43 (76.1 mg, 0.23 mmol) in CH₂Cl₂ (5 mL) was added mCPBA (173.2 mg, 0.70 mmol). The mixture was stirred at room temperature for 15 min. Add NaHCO₃ (sat. aq. solution), extract 3 times with CH₂Cl₂. Column purification on silica gel using EtOAc/heptane (50/50) gave sulfone intermediate compound 44 (73.2 mg, 88%) as an oil.

Mass spectrum: -CI m/z (assignment, relative intensity) 358 (MH⁺, 21%), 340 (MH⁺—H₂O, 9%), 330 (MH⁺—N₂, 9%), 303 (8%), 265 (24%), 264 (MH⁺—N₂—H₂SO₂, 25%), 237 (MH⁺—N₂—H₂SO₂—HCN, 11%), 211 (15%,), 197 (66%).

### EXAMPLE A42

### (10R,11R)-11-[(2S)-3-azido-2-hydroxypropyl]-8-fluoro-10,11-dihydro-5H-dibenzo-[a,d]cyclohepten-10-ol (intermediate compound 45)

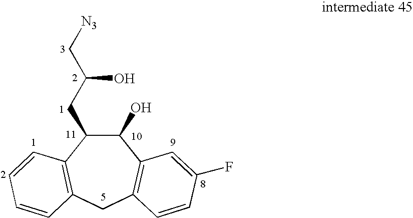

To a solution of intermediate compound 40 (0.85 g, 2.32 mmol) in THF (10 mL) was added PPh₃ (1.22 g, 4.63 mmol) and DIAD (1.92 mL, 4.63 mmol). Then, a solution of p-nitrobenzoic acid (0.77 g, 4.63 mmol) in THF (10 mL) was added dropwise. The mixture was stirred at room temperature for 2 hours. Work up by adding NH₄Cl (sat. aq. sol.), extract 3 times with CH₂Cl₂. Column chromatography purification on silica gel using CH₂Cl₂/heptane (70/30) gave the p-nitrobenzoate (inverted secondary OH group) as an oil (1.19 g, 99%). To a solution of this compound (2.01 g, 4.05 mmol) in MeOH (50 mL) was added K₂CO₃ (1.12 g, 8.10 mmol). The reaction mixture was stirred at room temperature for 3 hours. Add NH₄Cl (sat. aq. sol.), extract 3 times with CH₂C₁₂. Column purification on silica gel using EtOAc/heptane (30/70) gave an oily intermediate compound 45 (0.71 g, 98%).

### EXAMPLE A43

### S-((10S,11R)-11-{(2S)-3-azido-2-[(methylsulfonyl)oxy]propyl}-8-fluoro-10,11-dihydro-5H-dibenzo[a,d]cyclohenten-10-yl) ethanethioate (intermediate compound 46)

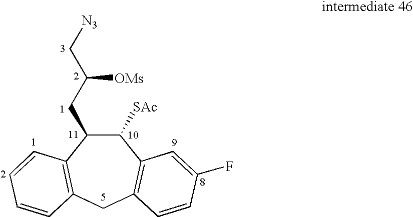

To a solution of intermediate compound 45 (1.20 g, 3.66 mmol) in CH₂Cl₂ (30 mL) was added Et₃N (4.10 mL, 29.3 mmol), DMAP (0.22 mg, 1.83 mmol) and (CH₃SO₂)₂O (1.92 g, 11.0 mmol) at 0° C. Stir at room temperature for 1 hour. Cool to 0° C. again and add AcSH (0.52 mL, 7.33 mmol) and stir at room temperature for 5 hours. Work up by adding NH₄Cl (sat. aq. sol.), extract 3 times with CH₂Cl₂ and dry with MgSO₄. Column chromatography purification on silica gel using EtOAc/heptane (30/70) afforded intermediate compound 46 as an oil (1.32 g, 78%).

### EXAMPLE A44

### (2R,3aR,12bS)-2-(azidomethyl)-11-fluoro-3,3a,8,12b-tetrahydro-2H-dibenzo[3,4:6,7]-cyclohepta[1,2-b]thiophene (intermediate compound 47)

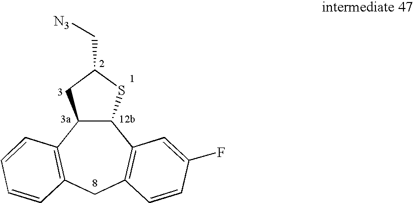

To a solution of intermediate compound 46 (1.32 g, 2.86 mmol) in MeOH (30 mL) was added K₂CO₃ (0.79 g, 5.72 mmol). After stirring at room temperature for 2 hours, NH₄Cl (sat. aq. sol.) was added. Extract 3 times with CH₂Cl₂ and dry with MgSO₄. Column chromatography purification on silica gel using CH₂Cl₂/heptane (40/60) gave intermediate compound 47 as an oily product (0.82 g, 89%).

Mass spectrum: -CI m/z (assignment, relative intensity)326 (MH+, 25%), 298 (MH⁺—N₂, 60%), 283 (MH⁺—HN₃, 100%), 269 (MH⁺—N₂—CH₂NH, 12%), 269 (MH⁺—HN₃—H₂S, 25%), 235 (MH⁺—N₂—CH₂NH—H₂S, 21%), 197 (61%).

### EXAMPLE A45

### (2R,3aR,12bS)-2-(azidomethyl)-11-fluoro-3,3a,8,12b-tetrahydro-2H-dibenzo[3,4:6,7]-cyclohepta[1,2-b]thiophene 1,1-dioxide (intermediate compound 48)

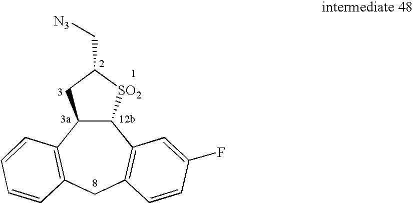

To a solution of intermediate compound 47 (136.1 mg, 0.41 mmol) in CH₂Cl₂ (10 mL) was added mCPBA (310.0 mg, 1.26 mmol). The mixture was stirred at room temperature for 30 minutes. Add NaHCO₃ (sat. aq. solution), extract 3 times with CH₂Cl₂. Column chromatography purification on silica gel using EtOAc/heptane (50/50) gave intermediate compound 48 (146.5 mg, 98%) as an oil.

Mass spectrum: -CI m/z (assignment, relative intensity)358 (MH⁺, 21%), 340 (MH⁺—H₂O, 9%), 330 (MH⁺—N₂, 9%), 303 (8%), 265 (24%), 264 (MH⁺—N₂—H₂SO₂, 25%), 237 (MH⁺—N₂—H₂SO₂—HCN, 11%), 211 (15%), 197 (66%).

### EXAMPLE A46

### (2S,3aR,12bS)-2-(azidomethyl)-11-fluoro-3,3a,8,12b-tetrahydro-2H-dibenzo[3,4:6,7]-cyclohepta[1,2-b]thiophene 1-oxides (intermediate compounds 49, 50)

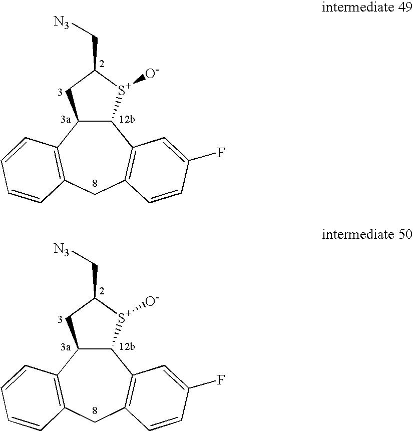

To a solution of intermediate compound 43 (0.34 g, 1.05 mmol) in HFIP (5 mL) was added H₂O₂ (30%, 0.24 mL, 2.10 mmol). The mixture was stirred at room temperature for 30 minutes. Add Na₂CO₃ (sat. aq. solution), extract 3 times with CH₂Cl₂. Column chromatography purification on silica gel using Et₂O (100%) afforded intermediate compounds 49 (110 mg) and 50 (130 mg) with a total yield of 78%.

Mass spectrum: -CI m/z (assignment, relative intensity) 342 (MH⁺, 100%), 314 (MH⁺—N₂, 49%), 299 (MH⁺—HN₃, 47%), 264 (17%), 197 (96%).

### EXAMPLE A47

### (2R,3aR,12bS)-2-(azidomethyl)-11-fluoro-3,3a,8,12b-tetrahydro-2H-dibenzo[3,4:6,7]-cyclohepta[1,2-b]thiophene 1-oxides (intermediate compounds 51, 52)

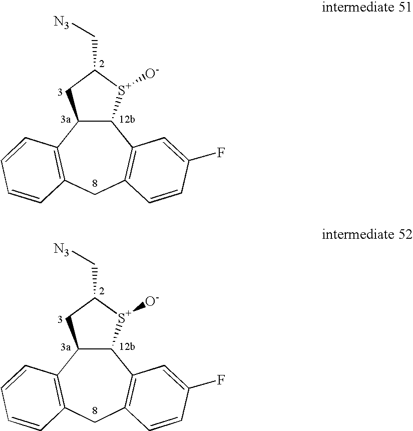

To a solution of intermediate compound 47 (0.21 g, 0.64 mmol) in HFIP (3 mL) was added H₂O₂ (30%, 0.15 μL, 1.27 mmol). The mixture was stirred at room temperature for 30 minutes. Add Na₂CO₃ (sat. aq. solution), extract 3 times with CH₂Cl₂. Column purification on silica gel using Et₂O (100%) gave intermediate compound 51 (120 mg) and 52 (86 mg) with a total yield of 95%.

Mass spectrum: -CI m/z (assignment, relative intensity) 342 (MH⁺, 100%), 314 (MH⁺—N₂, 49%), 299 (MH⁺—HN₃, 47%), 264 (17%), 197 (96%).

### EXAMPLE A48

### (10S*,11R*)-11-allyl-8-fluoro-10,11-dihydro-5H-dibenzo[a,d]cyclohepten-10-yl acetate (intermediate compound 56)

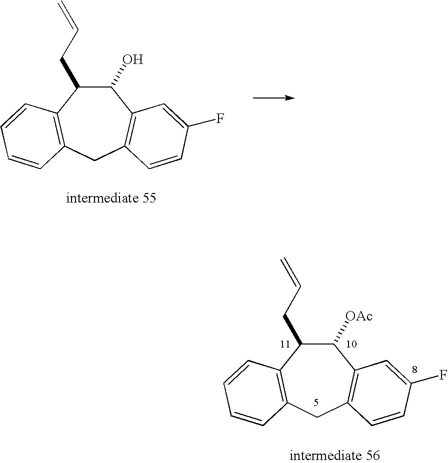

Dissolve intermediate compound 55 (1.72 g, 6.42 mmol) in CH₂Cl₂ (30 mL). Add Et₃N (1.79 mL, 12.8 mmol), DMAP (0.78 g, 6.42 mmol) and Ac₂O (1.21 mL, 12.8 mmol). Stir at room temperature for 1 hour and add sat. aq. NH₄Cl (15 mL). Extract 3 times with CH₂Cl₂ (3×20 mL) and dry with MgSO₄. Column chromatography purification on silica gel using CH₂Cl₂/hexane (60:40) yielded intermediate compound 56 as an oil (1.77 g, 89%).

Mass spectrum: -CI m/z (assignment, relative intensity)311 (MH⁺, 5%), 251 (MH⁺-AcOH, 100%); EI: m/z (assignment, relative intensity) 250 (M⁺-AcOH, 16%), 209 (M⁺-AcOH-CH₂CH₂—CH₂, 100%); High resolution EI Calculated C₁₈H₁₅F (M⁺-AcOH): 250.1158, Found: 250.1162 (26%).

### EXAMPLE A49

### a) (2R)-1-[(10R,11S)-11-azido-2-fluoro-10,11-dihydro-5H-dibenzo[a,d]cyclohepten-10-yl]-3-(trityloxy)-2-propanol (intermediate compound 57)

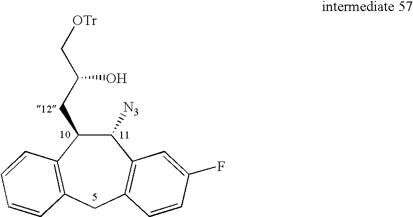

A mixture of intermediate compound 5 (6.022 g, 18.40 mmol), Et₃N (5.586 g, 55.2 mmol), DMAP (138 mg, 1.13 mmol), TrBr (9.444 g, 27.6 mmol) in CH₂Cl₂ (180 mL) was stirred at room temperature under nitrogen atmosphere for 2 hours, then quenched with sat. aq. NH₄Cl (50 mL). The organic phase was separated, aqueous layer extracted with CH₂Cl₂ (2×50 mL), combined organics washed with water (3×40 mL), brine (40 mL), dried (MgSO₄) and evaporated in vacuo. Purification by flash column chromatography (Kieselgel 60, 230-400 mesh, EtOAc-heptane, 5/95 to 10/90) gave intermediate compound 57 (8.595 g, 15.09 mmol, 82%) as a brown semisolid.

### b)(1R)-2-[(10R,11S)-11-Azido-2-fluoro-10,11-dihydro-5H-dibenzo[a,d]cyclohepten-10-yl]-1-[(trityloxy)methyl]ethylmethanesulfonate (intermediate compound 58)

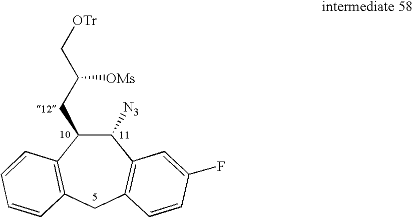

A mixture of intermediate compound 57 (8.5 g, 14.92 mmol), Et₃N (4.529 g, 44.76 mmol) and DMAP (84 mg, 0.689 mmol) in CH₂Cl₂ (200 mL) was cooled down to −78° C. under N₂ atmosphere. MsCl (2.264 g, 22.38 mmol) was added in one portion, resulting solution slowly warmed up to room temperature (ca. 40 min) and quenched with sat. aq. NH₄Cl (50 mL). The organic phase was separated, aqueous layer extracted with CH₂Cl₂ (3×45 mL), combined organics washed with water (3×45 mL) and brine (40 mL), dried (MgSO₄), and evaporated in vacuo. Due to the instability of intermediate compound 58 it was used immediately without further purification.

### c) (1R)-2-[(10R,11S)-11-azido-2-fluoro-10,11-dihydro-5H-dibenzo[a,d]cyclohepten-10-yl]-1-(hydroxymethyl)ethyl methanesulfonate (intermediate compound 59)

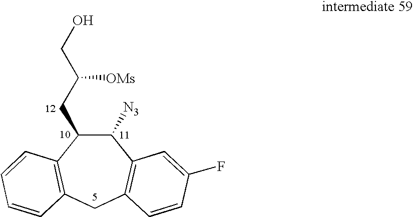

Crude intermediate compound 58 (unknown amount, assumed 14.92 mmol), was dissolved in MeOH (200 mL), dry Amberlyst-15 (15 g) added and the mixture was stirred at 45° C. for 4 hours; progress of reaction followed by TLC (Kieselgel on glass; EtOAc-heptane 30/70). The resin was filtered off and washed with MeOH (2×40 mL), methanolic solution concetrated in vacuo to 100 mL, and intermediate compound 59 used immediately for the next step.

### d) (10S,11R)-8-fluoro-11-[(2S)-oxiranylmethyl]-10,11-dihydro-5H-dibenzo[a,d]cyclo-hepten-10-yl azide (intermediate compound 60)

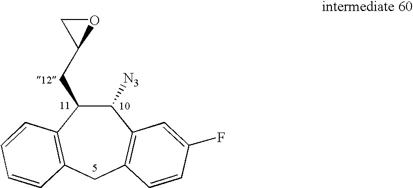

Methanolic solution of intermediate compound 59, obtained as above, was treated with anhydrous K₂CO₃ (4.146 g, 30 mmol) and stirred at room temperature for 3 hours. After treatment with water (100 mL), MeOH was removed in vacuo, product extracted with Et₂O (3×75 mL). The combined organics were washed with water (3×75 mL) and brine (40 mL), dried (MgSO₄) and evaporated in vacuo. Chromatographic purification (Kielselgel 60, 70-230 mesh, EtOAc-heptane 10/90) gave intermediate compound 60 (3.185 g, 10.29 mmol, 69% from intermediate compound 57) as a colorless oil. HRMS: Calcd. for C₁₈H₁₆FN₃O: 309.1277; Found: 309.1279.

### e) [(2R,3aR,12bS)-11-fluoro-1,2,3,3a,8, 1 2b-hexahydrodibenzo[3,4:6,7]cyclohepta[1,2-b]pyrrol-2-yl]methanol (intermediate compound 61)

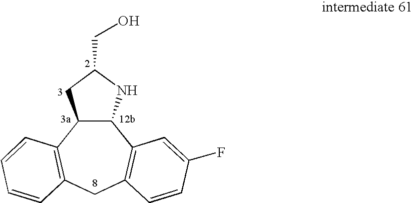

Intermediate compound 60 (3.108 g, 10.04 mmol) was dissolved in MeOH (50 mL), Et₃N (1.012 g, 10 mmol) and 10% Pd—C (150 mg) added, and resulting mixture hydrogenated under atmospheric pressure for 5 hours. Catalyst was removed by filtration through a short pad of Kieselguhr, MeOH and Et₃N removed in vacuo, and residue purified by column chromatography (Kieselgel 60, 70-230 mesh, EtOH—CH₂Cl₂ 5/95) to yield intermediate compound 61 (2.333 g, 8.23 mmol, 82%) as a yellowish oil, slowly solidifying on standing.

HRMS: Calcd. for C₁₈H₁₈FNO: 283.1372; Found: 283.1380.

### f) Methyl (2R,3aR,12bS)-11-Fluoro-2-(hydroxymethyl)-3,3a,8,12b-tetrahydrodibenzo-[3,4:6,7]cyclohepta[1,2-b]pyrrole-1(2H)-carboxylate (intermediate compound 62)

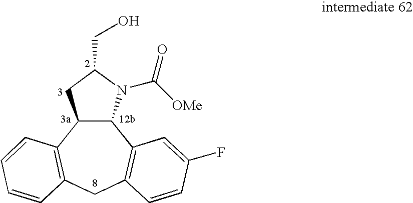

Intermediate compound 61 (567 mg, 2.00 mmol) was dissolved at 0° C. in a mixture of CH₂Cl₂ (20 mL) and sat. aq. NaHCO₃ (20 mL), then methyl chloroformate (0.23 mL, 281 mg, 2.98 mmol) was added, ice bath removed, and resulting mixture stirred for 5 hours. The organic layer was separated, aqueous phase extracted with CH₂Cl₂ (40 mL) then the combined organics washed with water (2×40 mL), brine (20 mL), dried (MgSO₄) and vaporated. The residue was purified by column chromatography on silica (CH₂Cl₂-EtOH, 95/5) to give carbamate intermediate compound 62 (669 mg, 1.96 mmol, 98%) as tan oil, solidifying on standing.

HRMS: Calcd. for C₂₀H₂₀FNO₃: 341.1427; Found: 341.1435.

### g) (2R,3aR,12bS)-11-Fluoro-2-(hydroxymethyl)-3,3a,8,12b-tetrahydrodibenzo-[3,4:6,7]cyclohepta[1,2-b]pyrrole-1(2H)-carbaldehyde (intermediate compound 62a)

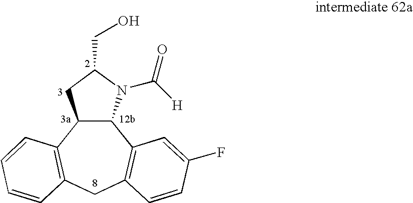

A mixture of intermediate compound 61 (283 mg, 1 mmol), ethyl formate (741 mg, 10 mmol) and acetonitrile (10 mL) was refluxed for 18 hours, then evaporated in vacuo. The residue was purified by chromatography (Kieselgel 60, 70-230 mesh, EtOH—CH₂Cl₂ 5/95) to yield 62a (283 mg, 0.91 mmol, 91%) as a tan solid.Product is mixture of 2 rotamers (3:2 ratio).

CI-MS (CH₄) 312 (MH⁺, 100%); 292 (MH⁺—HF, 13%).

### EXAMPLE A50

### (2R,3aR,12bS)-11-Fluoro-3,3a,8,12b-tetrahydrodibenzo[3,4:6,7]cyclohepta[1,2-b]-pyrrole-1,2(2H)-dicarbaldehyde (intermediate compound 63)

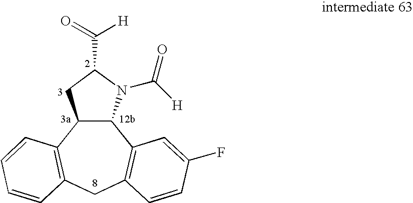

PCC (104 mg, 0.48 mmol) was added to the solution of intermediate compound 62a (100 mg, 0.32 mmol) in CH₂Cl₂ (10 mL), and the resulting slurry was stirred under N₂ atmosphere for 3 hours. After addition of Et₂O (20 mL), the mixture was filtered through silica gel, the tar residue in flask washed with Et₂O (40 mL), filtered again, the filtrates were evaporated to dryness in vacuo. Crude intermediate compound 63 (77 mg, 0.25 mmol, 78%) was obtained as reddish oil, containing traces of chromium. Product was used immediately without purification.

CI-MS (CH₄) 310 (100%, MH⁺), 290 (11%, MH⁺—HF); 282 (7%, MH⁺—CO).

### EXAMPLE A51

### (2aS,11bR,12aR)-4-Fluoro-1,2a,7,11b,12,12a-hexahydroazireno[1,2-a]dibenzo-[3,4:6,7]cyclo-hepta[1,2-d]pyrrole (intermediate compound 64)

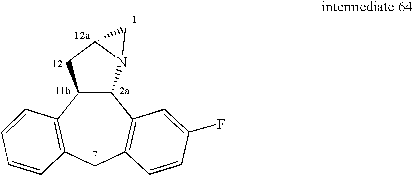

Polymer supported triphenylphosphine (0.33 g, ca. 1 mmol of PPh₃) was swollen under argon atmosphere in dry CH₂Cl₂ (10 mL), then DIAD (222 mg, 1.1 mmol) in THF (3 mL) was added through septum at 0° C. The suspension was stirred for 30 minutes at 0° C., followed by addition of intermediate compound 61 (104 mg, 0.366 mmol) in THF (4 mL). The cooling bath was removed and reaction mixture was stirred at room temperature for 12 hours, then water (0.1 mL) was added, resin filtered off and washed with THF (15 mL), combined organics evaporated and purified by column chromatography (Kieselgel 60, 230-400 mesh, CH₂Cl₂-EtOH 100/0 to 96/4) to give intermediate compound 64 (63 mg, 0.238 mmol, 65%) as yellowish oil.

HRMS Calcd. for C₁₈H₁₆FN: 265.1267; Found: 265.1270.

### EXAMPLE A52

### a) {(2R,3aR,12bS)-11-Fluoro-1-[(2-nitrophenyl)sulfonyl]-1,2,3,3a,8,12b-hexahydrodibenzo[3,4:6,7]cyclohepta[1,2-b]pyrrol-2-yl}methyl 2-nitrobenzenesulfonate (intermediate compound 65)

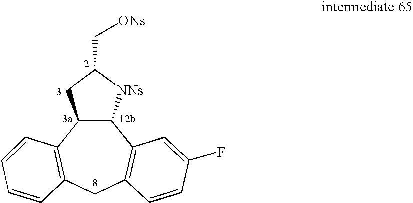

A solution of intermediate compound 61 (567 mg, 2.00 mmol), Et₃N (1.012 mmol, 10.00 mmol), DMAP (40 mg, 0.33 mmol) in CH₂Cl₂ (30 mL) was treated with NsCl (1.330 g, 6.00 mmol), and resulting mixture was stirred at room temperature for 3 hours, then quenched with sat. aq. NH₄Cl (30 mL). After extraction with CH₂Cl₂ (3×30 mL) the combined organics were washed with 1N HCl (15 mL), sat. aq. K₂CO₃ (40 mL), water (3×40 mL), brine, dried (MgSO₄), evaporated and purified by column chromatography on silica (heptane-EtOAc, 95/543 85/15) to give intermediate compound 65 (1.203 g, 1.84 mmol, 92%) as yellow crystals, rapidly decomposing on standing.

¹HNMR (300 MHz, CDCl₃) δ 8.26-7.50 (m, 8H, Ar—H, 2-nosyl moieties); 7.20-7.03 (m, 6H, Ar—H, dibenzosuberone part); 6.81 (td, J=8.3, 2.7 Hz, 1H, Ar—H); 5.40 (d, J=11.0 Hz, 1H, CH-12b); 4.69 (d, J=16.7 Hz, 1H, CH₂-8); 4.60 (m, 2H, CH₂ONs); 4.40 (m, 1H, CH-2); 3.73 d, J=16.7 Hz, 1H, CH₂′-8); 3.55-3.40 (m, 1H, CH-3a); 2.80 (dd, J=13.0, 6.2 Hz, CH₂-3); 2.33-2.18 (m, 1H, CH₂′-3).

### b) 2-[4-({(2R,3aR,12bS)-11-Fluoro-1-[(2-nitrophenyl)sulfonyl]-1,2,3,3a,12b-hexa-hydro-dibenzo[3,4:6,7]cyclohepta[1,2-b]pyrrol-2-yl}methyl)-1-piperazinyl]ethanol intermediate compound 66a) and 2-[({(2R,3aR,12bS)-11-Fluoro-1-[(2-nitrophenyl)-sulfonyl]-1,2,3,3a,8,12b-hexahydrodibenzo-[3,4:6,7]cyclohepta[1,2-b]pyrrol-2-yl}-methyl)(methyl)amino]ethanol (intermediate compound 66b)

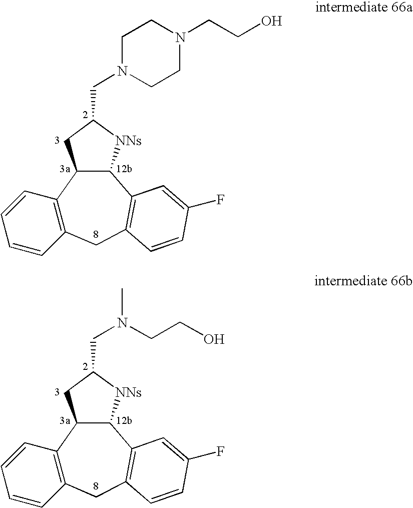

A mixture of intermediate compound 65 (0.35 g, 0.54 mmol) and the appropriate amine (3 mmol) in dioxane (10 mL) was refluxed for 4 hours, cooled down to ambient temperature, diluted with water (100 mL), precipitated product filtered off, washed with water (100 mL), dissolved in EtOAc, solution washed with brine, dried (K₂CO₃), evaporated, and used for next step without purification.

### EXAMPLE A53

### a) 2-[(4S)-2,2-Diethyl-1,3-dioxolan-4-yl]ethanol (intermediate compound 67b)

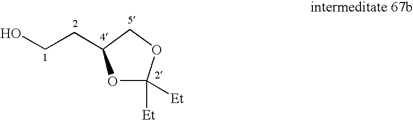

To a solution of (S)-1,2,4-butanetriol intermediate compound 67a (6.76 g, 63.68 mmol) in freshly distilled pentan-3-one (320 mL) was added p-toluenesulfonic acid (p-TSA) (6.06 g, 31.84 mmol). The reaction mixture was stirred at 53° C. for 16 hours, then Et₃N (10 mL) was added and the reaction mixture stirred at ambient temperature for 10 minutes. The reaction mixture was concentrated under reduced pressure. Gradient flash chromatography (CH₂Cl₂/MeOH, 100:0 to 97:3 to 95:5) afforded the protected alcohol intermediate compound 67b (9.84 g, 89%) as a colorless oil.

### b) [(4′S)-2,2-Diethyl-1,3-dioxolan-4-yl]acetaldehyde (intermediate compound 67c)

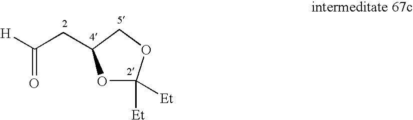

To a solution of 2 intermediate compound 67b (4.00 g, 22.96 mmol) and 4A molecular sieves (11.50 g) in CH₂Cl₂ (200 mL) stirred at 0° C. for 5 minutes was added PCC (9.90 g, 45.92 mmol). The reaction mixture was allowed to warm to ambient temperature and stirred for 1 hour. The crude reaction mixture was filtered through a pad of silica gel, washed with Et₂O (50 mL) and concentrated under reduced pressure to afford the intermediate compound 67c (3.56 g, 90%) as a colorless oil.

### c) 11-{2-[(4S)-2,2-diethyl-1,3-dioxolan-4-yl]ethylidene}-8-fluoro-5,11-dihydro-10H-dibenzo-[a,d]cyclohepten-10-one (intermediate compound 67d)

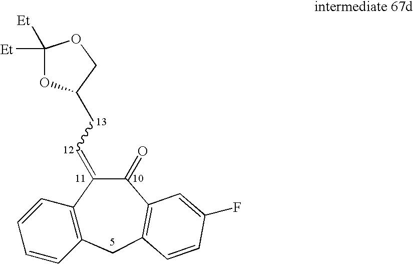

MgBr₂ (0.733 g, 3.98 mmol) was added to 8-fluoro-5,11-dihydro-10H-dibenzo[a,d]-cyclohepten-10-one (0.75 g, 3.32 mmol) in toluene (15 mL) and the reaction mixture stirred at room temperature for 30 minutes. Intermediate compound 67c (2.05 g, 11.92 mmol) in THF (10 mL) was added and in one time t-BuOK (0.074 g, 0.66 mmol). The reaction mixture was stirred for 22 hours at ambient temperature, then sat. aq. NH₄Cl (15 mL) was added. The product was extracted three times with Et₂O (3×30 mL), combined organics washed with water (2×35 mL), brine (25 mL) dried over MgSO₄. After evaporation of the toluene, the residue was purified on silica gel column using Et₂O/heptane (10/90) to obtain intermediate compound 67d (1.079 g, 86%) as a yellowish oil.

HRMS (EI) Calcd. for C₂₄H₂₅FO₃: 380.1800; Found: 380.1785.

### d) (11R)-11-{2-[(4S)-2,2-diethyl-1,3-dioxolan-4-yl]ethyl}-8-fluoro-5,11-dihydro-10H-dibenzo-[a,d]cyclohepten-10-one (intermediate compound 67e)

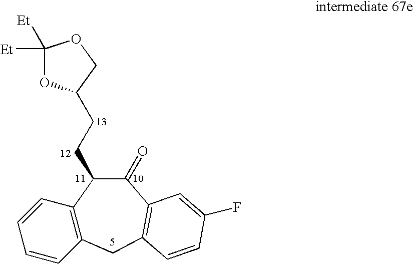

10% Pd—C (200 mg) and Et₃N (0.135 mL, 0.97 mmol) were added to intermediate compound 67d (0.246 g, 0.647 mmol) in i-PrOH (25 mL) and toluene (15 mL) and subjected to hydrogenation overnight at room temperature. The reaction mixture was dissolved in CH₂Cl₂, filtered through celite and solvent evaporated. The residue was purified by column chromatography on silica gel using Et₂O/heptane (30/70) to give intermediate compound 67e (151 mg, 61%) as a yellowish oil.

HRMS C₂₄H₂₇FO₃: 382.1944; Found: 382.1951.

### e) (10R,11R)-1-{2-[(4S)-2,2-diethyl-1,3-dioxolan-4-yl]ethyl}-8-fluoro-10,11-dihydro-5H-di-benzo[a,d]cyclohepten-10-ol (intermediate compound 67f)

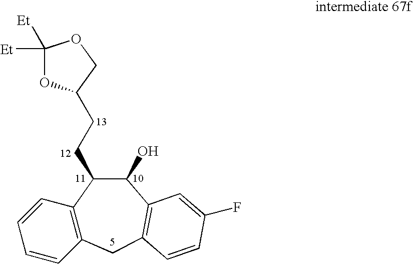

NaBH₄ (1.78 g, 46.84 mmol) was added to intermediate compound 35 (2.0 g, 5.88 mmol) dissolved in i-PrOH (80 mL)/pH 7 phosphate buffer (30rnL) at 0° C. After 1 hour of reaction at room temperature, NH₄Cl (sat. aq. solution) was added and the mixture was extracted three times with CH₂Cl₂. The organic phase was dried over MgSO₄ and the solvent evaporated. The product was purified by column chromatography on silica gel using Et₂O/heptane (30/70) which gave intermediate compound 67f (1.96 g, 97%) as colorless oil.

HRMS Calcd. for C₂₁H₂₃FO₃: 342.1631; Found: 342.1627.

### f) (4S)-4-{2-[(10R,11S)-11-azido-2-fluoro-10,11-dihydro-5H-dibenzo[a,d]cyclohepten-10-yl]-ethyl}-2,2-diethyl-1,3-dioxolane (intermediate compound 67g)

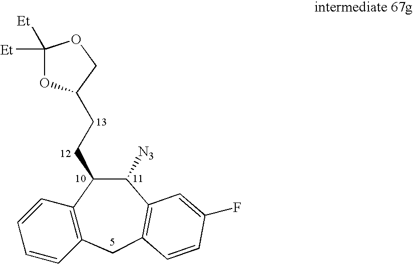

Intermediate compound 67g was obtained in the same way as described for intermediate compound 30.

### g) (2S)-4-[(10R,11S)-11-azido-2-fluoro-10,11-dihydro-5H-dibenzo[a,d]cyclohepten-10-yl]-1,2-butanediol (intermediate compound 67h)

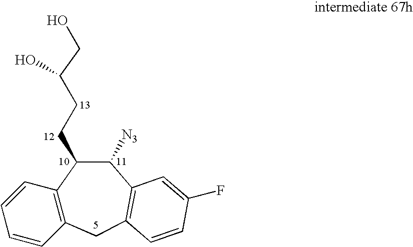

Intermediate compound 67h was obtained in the same way as described for intermediate compound 31.

### h) (2S)-4-[(10R,11S)-11-azido-2-fluoro-10,11-dihydro-5H-dibenzo[a,d]cyclohepten-10-yl]-2-{[(4-methylphenyl)sulfonyl]oxy}butyl 4-methylbenzenesulfonate (intermediate compound 67i)

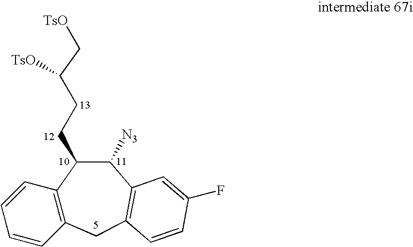

A mixture of intermediate compound 67h (225 mg, 0.66 mmol), Et₃N (506 mg, 5.0 mmol), DMAP (12 mg, 0.1 mmol) and TsCl (503 mg, 2.64 mmol) in CH₂Cl₂ (25 mL) was stirred at room temperature under nitrogen atmosphere for 15 hours. After quenching with sat. aq. NH₄Cl (15 mL), the organic phase was separated, and the aqueous layer extracted with CH₂Cl₂ (3×20 mL). The combined organics were washed with water (3×30 mL), brine (25 mL), dried over MgSO₄ and evaporated in vacuo. The residue was purified by column chromatography (Kieselgel 60, 70-230 mesh, heptane-EtOAc 90/10) to afford intermediate compound 67i (352 mg, 0.54 mmol, 82%) as colorless semi-solid.

### i) [(2R,4aR,13bS)-12-Fluoro-2,3,4,4a,9,13b-hexahydro-1H-dibenzo[3,4:6,7]cyclohepta[1,2-b]pyridin-2-yl]methyl 4-methylbenzenesulfonate (intermediate compound 67k)

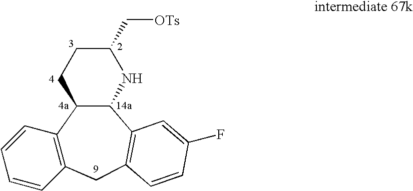

Intermediate compound 67i (340 mg, 0.52 mmol) was dissolved in MeOH (15 mL), Et₃N (1.012 g, 10 mmol) and 10% Pd—C (150 mg) added, and resulting mixture hydrogenated under atmospheric pressure for 5 hours. Catalyst was removed by filtration through short pad of Kieselguhr, anhydrous K₂CO₃ (138 mg, 1 mmol) added and resulting slurry stirred at room temperature for 5 hours. After filtration of solids, MeOH and Et₃N were removed in vacuo, and residue purified by flash chromatography (Kieselgel 60, 230-400 mesh, EtOH—CH₂Cl₂ 5/95 to 12/88) to yield intermdiate 67k (153 mg, 0.338 mmol, 65%) as yellowish oil.

Cl-MS (CH₄) 452 (MH⁺, 1%); 280 (MH⁺-TsOH, 100%).

### EXAMPLE A54

### a) (10S,11R)-11-{2-[(4S)-2,2-diethyl-1,3-dioxolan-4-yl]ethyl}-8-fluoro-10,11-dihydro-5H-dibenzo[a,d]cyclohepten-10-yl 4-nitrobenzoate (intermediate compound 68a)

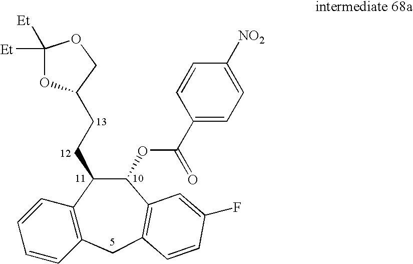

A solution of PPh₃ (910 mg, 3.5 mmol) in dry THF (25 mL) was placed in a two-necked 100 mL flask, equipped with septum, argon inlet and magnetic stirrer. After cooling down to −15° C. neat DIAD (708 mg, 3.5 mmol) was added through a septum with intensive stirring. Resulting yellow suspension was stirred at above temperature for 30 minutes, then a solution of 4-nitrobenzoic acid (585 mg, 3.50 mmol) and alcohol intermediate compound 67f (673 mg, 1.75 mmol) in THF (25 mL) was added within 10 minutes. The resulting yellow suspension was allowed to warm up to room temperature and stirred then for 12 hours. Water (0.3 mL) was added, followed by silica gel (Kieselgel 60, 70-230 mesh, 4 g), THF removed in vacuo, and the remaining silica powder was purified by flash column chromatography (Kieselgel 60, 230-400 mesh, EtOAc-heptane, 5/95 to 15/85) to give nitrobenzoate intermediate compound 68a (795 mg, 1.49 mmol, 85%) as an orange semisolid.

### b) (10S,11R)-11-[(3S)-3,4-dihydroxybutyl]-8-fluoro-10,11-dihydro-5H-dibenzo[a,d]-cyclohepten-10-yl 4-nitrobenzoate (intermediate compound 68b)

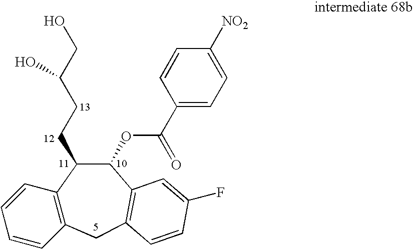

Intermediate compound 68a (795 mg, 1.49 mmol) was carried out in the same was as described in Example A28 to give diol intermediate compound 68b (695 mg, 1.49 mmol, 100%) as orange semisolid. Product intermediate 68b was used without purification.

### c) (10S,11R)-8-fluoro-11-((3S)-3-hydroxy-4-{[(4-methylphenyl)sulfonyl]oxy}butyl)-10,11-dihydro-5H-dibenzo[a,d]cyclohepten-10-yl 4-nitrobenzoate (intermediate compound 68c)

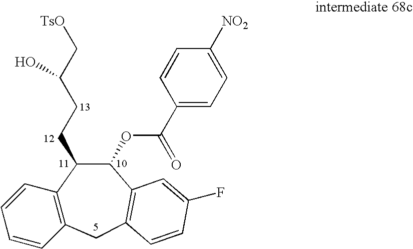

Intermediate compound 68b (695 mg, 1.49 mmol), Et₃N (607 mg, 6 mmol), dibutyl-(oxo)stannane (141 mg, 0.566 mmol), and TsCl (431 mg, 2.26 mmol) in CH₂Cl₂ (20 mL) was stirred at room temperature under N₂ for 12 hours. After quenching with sat. aq. NH₄Cl (15 mL) the organic phase was separated and the aqueous solution extracted with CH₂Cl₂ (3×30 mL). The combined organics were washed with water (3×20 mL), filtered through 5 cm layer of MgSO₄, and evaporated in vacuo to furnish crude intermediate compound 68c (601 mg, 0.97 mmol, 65%) as a yellowish semisolid mass, which was used without further purification.

### d) [(2R,4aR,13bS)-12-fluoro-2,3,4,4a,9,13b-hexahydrodibenzo[3,4:6,7]cyclohepta-[1,2-b]pyran-2-yl]methanol (intermediate compound 68d)

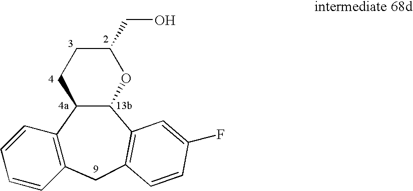

A mixture of intermediate compound 68c (601 mg, 0.97 mmol), sodium methoxide (162 mg, 3.0 mmol) and MeOH (10 mL) was stirred at room temperature for 3 hours. After treatment with water (100 mL) product was extracted with Et₂O (3×30 mL). The combined organics were washed with water (3×40 mL) and brine (40 mL), dried (MgSO₄) and evaporated in vacuo. Chromatographic purification (Kielselgel 60, 70-230 mesh, EtOAc-heptane 10/90 to 25/75) gave intermediate compound 68d (211 mg, 0.71 mmol, 73%) as a colorless oil.

HRMS Calcd. for C₁₉H₁₉FO₂: 298.1369; Found 298.1350.

### d) [(2R,4aR,13bS)-12-fluoro-2,3,4,4a,9,13b-hexahydrodibenzo[3,4:6,7]cyclohepta-[1,2-b]pyran-2-yl]methyl 4-methylbenzenesulfonate (intermediate compound 68e)

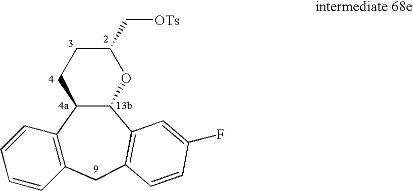

A mixture of intermediate compound 68d (211 mg, 0.708 mmol), Et₃N (209 μL, 287 mg, 2.83 mmol), DMAP (86.5 mg, 0.708 mmol), and TsCl (270 mg, 1.42 mmol) in CH₂Cl₂ (10 mL) was stirred at room temperature under N₂ for 16 hours. After quenching with sat. aq. NH₄Cl (10 mL) the organic phase was separated and the aqueous solution extracted with CH₂Cl₂ (3×15 mL). The combined organics were washed with water (3×15 mL), dried (MgSO₄), and evaporated in vacuo to give crude intermediate compound 68e (282 mg, 0.62 mmol, 88%) as a yellowish oil, which was used without further purification.

### EXAMPLE A55

### a) (10S,11R)-11-{[(4R)-2,2-dimethyl-1,3-dioxolan-4-yl]methyl}-8-fluoro-10,11-dihydro-5H-dibenzo[a,d]cyclohepten-10-yl 4-nitrobenzoate (intermediate compound 69a)

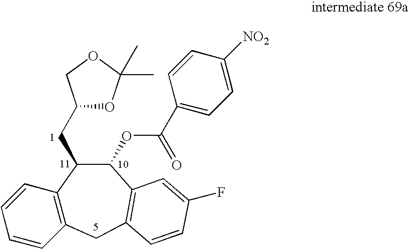

A solution of PPh₃ (1049 mg, 4.0 mmol) in dry THF (20 mL) was placed in a two-necked 100 mL flask, equipped with septum, argon inlet and magnetic stirrer. After cooling down to −15° C. neat DIAD (809 mg, 4.0 mmol) was added through a septum with intensive stirring. Resulting yellow suspension was stirred at above temperature for 30 minutes, then a solution of 4-nitrobenzoic acid (4.0 mmol) and intermediate compound 3 (685. mg, 2.0 mmol) in THF (25 mL) was added within 10 minutes. The resulting yellow suspension was allowed to warm up to room temperature and stirred then for 12 hours. Water (0.3 mL) was added, followed by silica gel (Kieselgel 60, 70-230 mesh, 4 g), THF removed in vacuo, and the silica powder was submitted to the flash chromatography (Kieselgel 60, 230-400 mesh, EtOAc-heptane, 5/95 to 10/90) to give intermediate compound 69a (875 mg, 1.78 mmol, 89%) as an orange semisolid.

### b) (10S,11R)-11-[(2R)-2,3-dihydroxypropyl]-8-fluoro-10,11-dihydro-5H-dibenzo-[a,d]cyclo-hepten-10-yl 4-nitrobenzoate (intermediate compound 69b)

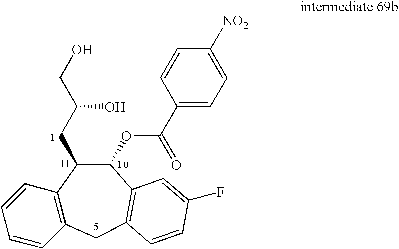

Intermediate compound 69b has been obtained from acetal intermediate compound 69a (860 mg, 1.75 mmol) in the same way as described for intermediate compound 5. Column chromatography (Kieselgel 60, 70-230 mesh, EtOAc-heptane, 35/65 to 50/50) afforded intermediate compound 69b (774 mg, 1.715 mmol, 98%) as a yellow semi-solid.

### c) (10S,11R)-8-fluoro-11-(2-oxoethyl)-10,11-dihydro-5H-dibenzo[a,d]cyclohepten-10-yl 4-nitrobenzoate (intermediate compound 69c)

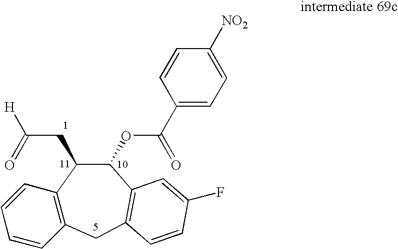

Intermediate compound 69b (774 mg, 1.715 mmol) was dissolved at 0° C. in a mixture of THF (25 mL) and pH 7 phosphate buffer (5 mL), then sodium periodate (642 mg, 3 mmol) was added in one portion at 0° C., cooling bath removed, and resulting mixture was stirred at room temperature for 4 hours. Water (50 mL) was added, product extracted with Et₂O (3×30 mL). The combined organics were washed with sat. aq. sodium metabisulfite (50 mL), water (2×50 mL), brine (30 mL), dried over MgSO₄ and evaporated in vacuo to give intermediate compound 69c (680 mg, 1.66 mmol, 97%) as a yellow foam. Product was used immediately without purification.

### d) (10S,11 S)-8-fluoro-11-(1-formylvinyl)-10,11-dihydro-5H-dibenzo[a,d]cyclohepten-10-yl 4-nitrobenzoate (intermediate compound 69d)

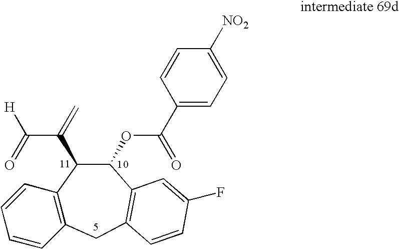

A mixture of intermediate compound 69c (680 mg, 1.66 mmol), AcOH (240 mg, 4.0 mmol), bis(dimethylamino)methane (719 mg, 7.0 mmol) and THF (30 mL) was stirred at room temperature for 3 hours. Water (50 mL) was added, product extracted with Et₂O (3×30 mL). The combined organics ware washed with sat. aq. NaHCO₃ (25 mL), water (2×50 mL), brine (30 mL), dried over MgSO₄ and evaporated in vacuo to give intermediate compound 69d (680 mg, 1.58 mmol, 95%) as a yellow oil.

### e) (10S,11S)-8-fluoro-11-[1-(hydroxymethyl)vinyl]-10,11-dihydro-5H-dibenzo[a,d]-cyclo-hepten-10-yl 4-nitrobenzoate (intermediate compound 69e)

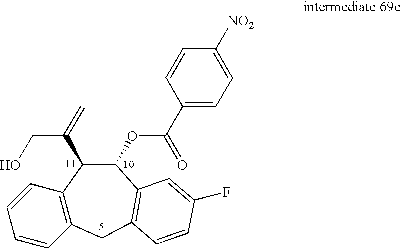

NaBH₄ (190 mg, 5.00 mmol) was added at room temperature within 10 minutes to the solution of intermediate compound 69d (680 mg, 1.58 mmol) in MeOH (30 mL). The reaction mixture was stirred at room temperature for 4 hours, quenched with sat. aq. NH₄Cl (20 mL) and extracted with Et₂O (3×30 mL). The combined organics ware washed with water (2×50 mL), brine (30 mL), dried over magnesium sulfate and evaporated in vacuo to give intermediate compound 69e (582 mg, 1.34 mmol, 85%) as an orange oil.

### f) (10S,11S)-8-fluoro-11-[1-(hydroxymethyl)vinyl]-10,11-dihydro-5H-dibenzo[a,d]-cyclo-hepten-10-ol (intermediate compound 69f)

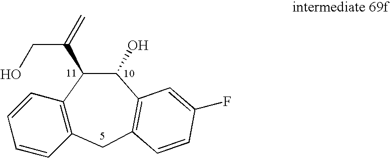

A mixture of intermediate compound 69e (582 mg, 1.34 mmol), sodium methoxide (162 mg, 3.0 mmol) and MeOH was stirred at room temperature for 4 hours. Water (70 mL) was added, product extracted with EtOAc (3×30 mL). The combined organics were washed with water (2×50 mL), brine (30 mL), dried over magnesium sulfate and evaporated in vacuo. The residue was purified by flash column chromatography (Kieselgel 60, 230-400 mesh, EtOAc-heptane, 35/65 to 60/40) to give intermediate compound 69f (286 mg, 1.005 mmol, 75%) as colorless oil.

### g) (3aS,12bS)-11-fluoro-3-methylene-3,3a,8,12b-tetrahydro-2H-dibenzo[3,4:6,7]cyclo-hepta-[1,2-b]furan (intermediate compound 69g)

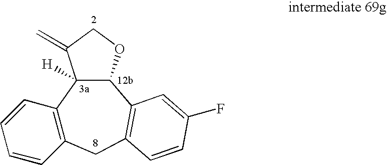

PBu₃ (405 mg, 2.0 mmol) was dissolved in toluene (25 mL) under argon atmosphere. DIAD (405 mg, 2.0 mmol) in toluene (3 mL) was added dropwise, followed by solution of intermediate compound 69f (270 mg, 0.95 mmol). The resulting mixture was stirred at room temperature for 3 hours, then reaction was quenched water (1 mL). Silica gel (Kieselgel 60, 70-230 mesh, 1.3 g) was added, toluene removed in vacuo, and silica powder submitted to the flash chromatography (Kieselgel 60, 230-400 mesh, EtOAc-heptane, 5/95 to 12/88) to give intermediate compound 69g (205 mg, 0.77 mmol, 81%) as colorless foam.

### h) [(3aR,12bS)-11-fluoro-3,3a,8,12b-tetrahydro-2H-dibenzo[3,4:6,7]cyclohepta[1,2-b]-furan-3-yl]methanol (intermediate compound 69h)

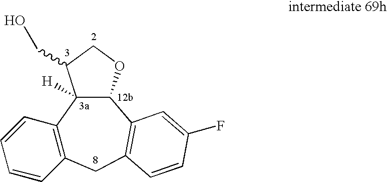

Boron trifloride etherate (0.43 mL, 3.54 mmol) in THF (1 mL) was added at room temperature under argon atmosphere to the solution of intermediate compound 69g (188 mg, 0.66 mmol), NaBH₄ (496 mg, 2.64 mmol) in dry THF (2 mL). The resulting solution was stirred under argon for 24 hours, excess of borohydrided decomposed carefully with water (3.8 mL), MeOH(1.5 mL) added, followed by 3M NaOH (3.8 mL) and 30% hydrogen peroxide (0.55 mL). Reaction mixture was allowed to stir for 4 hours at room temperature, then the product was extracted with Et₂O (3×30 mL). The combined organics ware washed with water (2×50 mL), brine (30 mL), dried over MgSO₄ and evaporated in vacuo. The residue was purified by flash chromatography (Kieselgel 60, 230-400 mesh, EtOAc-heptane, 5/95 to 20/80) to give THF derivative intermediate compound 69h (139 mg, 0.49 mmol, 74%) as colorless oil.

### i) (3aR,12bS)-3-(azidomethyl)-11-fluoro-3,3a,8,12b-tetrahydro-2H-dibenzo[3,4:6,7]-cyclohepta[1,2-b]furan (intermediate compound 69i)

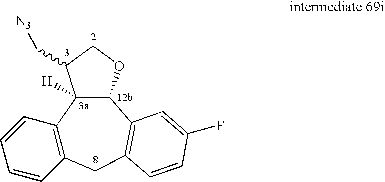

Polymer supported triphenylphosphine (0.33 g, ca. 1 mmol of PPh₃) was swollen at room temperature under argon atmosphere in dry THF (10 mL), then DIAD (222 mg, 1.1 mmol) in THF (3 mL) was added through septum at −15° C. The suspension was stirred for 30 minutes at −15° C., then alcohol intermediate compound 69h (139 mg, 0.49 mmol) in dry THF (2.5 mL) was added in one portion, followed by dropwise addition of DPPA (160 mg, 0.58 mmol) in THF (3 mL). Resulting suspension was stirred under argon for 12 hours. After quenching with water (0.3 mL), resin was filtered off and solvent removed in vacuo. The residue was purified by flash column chromatography (Kieselgel 60, 230-400 mesh, EtOAc-heptane, 15/85) to give intermediate compound 69i (136 mg, 0.44 mmol, 90%) as colorless foam.

### EXAMPLE A56

### a) (2R)-3-[(10R,11R)-2-fluoro-11-hydroxy-10,11-dihydro-5H-dibenzo[a,d]cyclohepten-10-yl]-1,2-propanediol (intermediate compound 70a)

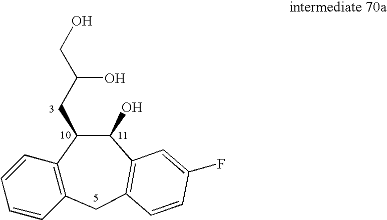

Triol intermediate compound 70a was obtained from intermediate compound 3 (514 mg, 1.50 mmol) in the same way as described in Example A4. Crude intermediate compound 70a (449 mg, 1.485 mmol, 99%) was obtained as colorless oil and used without purification.

### b) (3aR,12bR)-11-fluoro-3,3a,8,12b-tetrahydro-2H-dibenzo[3,4:6,7]cyclohepta[1,2-b]-furan-2-ol (intermediate compound 70b)

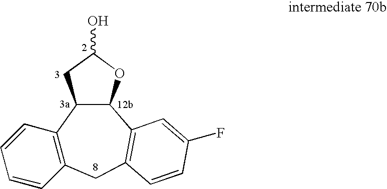

Intermediate compound 70b was obtained from intermediate compound 70a (449 mg, 1.485 mmol) in the same way as described for intermediate compound 44. Flash column chromatography (Kieselgel 60, 230-400 mesh, EtOAc-heptane, 10/90 to 33/67) afforded intermediate compound 70c (357 mg, 1.32 mmol, 89%) as a solid.

### c) (3aR,12bR)-3-[(dimethylamino)methyl]-11-fluoro-3,3a,8,12b-tetrahydro-2H-dibenzo-[3,4:6,7]cyclohepta[1,2-b]furan-2-ol (intermediate compound 70c)

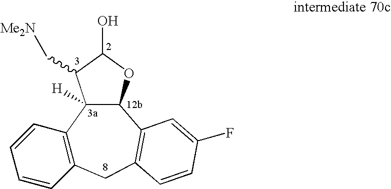

Reaction of intermediate compound 70c (335 mg, 1.24 mmol) was carried out in the same way as described for intermediate compound 45. Complex, unseparable mixture of products has been formed and used for the next step without purification.

### d) (10R,11R)-11-[2-(dimethylamino)-1-(hydroxymethyl)ethyl]-8-fluoro-10,11-dihydro-5H-dibenzo[a,d]cyclohepten-10-ol (intermediate compound 70d)

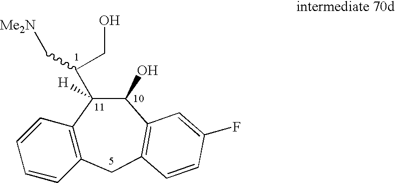

Reaction of the mixture containing intermediate compound 70c was was carried out in the same way as described for intermediate compound 46. Purification by RP-HPLC (Waters Xterra® C₁₈, 19×50 mm, MeOH-water 50/50, then pure MeOH, 4 mL/min) afforded intermediate compound 70d (135 mg, 0.41 mmol, 33% from intermediate compound 70b) as yellow oil.

### EXAMPLE A57

### a) [(10R,11S)-11-azido-2-fluoro-10,11-dihydro-5H-dibenzo[a,d]cyclohepten-10-yl]-acetaldehyde (intermediate compound 71a)

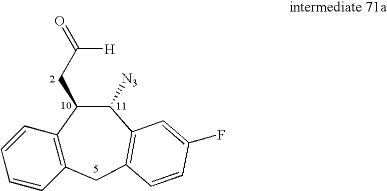

Reaction of diol intermediate compound 5 (0.99 g mg, 3.02 mmol) was carried out in the same way as described for intermediate compound 44. Purification by column chromatography (Kieselgel 60, 230-400 mesh, Et₂O-heptane, 50/50) gave intermediate compound 71a (778 mg, 2.63 mmol, 87%) as colorless oil.

### b) 2-[(10S,11S)-1-azido-2-fluoro-10,11-dihydro-5H-dibenzo[a,d]cyclohepten-10-yl]-acryl-aldehyde (intermediate compound 71b)

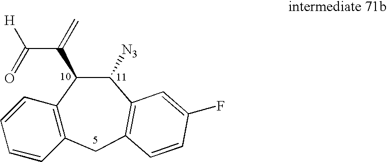

Reaction of intermediate compound 71a (618 mg, 2.09 mmol) was carried out in the same way as described for intermediate compound 45. Crude intermediate compound 71b (605 mg, 1.97 mmol, 94%) was obtained as colorless oil and was used without further purification.

### c) (3aS,12bS)-11-fluoro-3-methylene-1,2,3,3a,8,12b-hexahydrodibenzo[3,4:6,7]cyclo-hepta[1,2-b]-pyrrole (intermediate compound 71c)

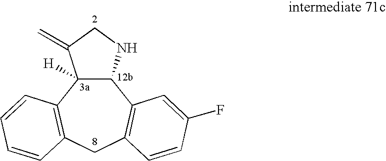

Polymer supported PPh₃ (1.40 g, ca. 4.2 mmol of PPh₃) was swollen at room temperature under argon atmosphere in THF (30 mL), then intermediate compound 71b (405 mg, 1.32 mmol) in THF (10 mL) and water (0.19 g) were added. The resulting mixture was stirred under argon at 50° C. for 1 hour. After this time resin was filtered off, THF remove in vacuo. The residue was dissolved in MeOH (10 mL), AcOH (1 mL) and NaCNBH₄ (200 mg, 3.2 mmol) added and resulting mixture stirred at room temperature for 2 hours, then quenched with concentrated HCl (1 mL), treated with sat. aq. NaHCO₃ (15 mL) and basified with 1N NaOH (3 mL). Product was extracted with CH₂Cl₂ (3×50 mL), combined organics washed with water (2×30 mL), brine (30 mL), dried (MgSO₄) and evaporated in vacuo to afford pyrrolidine intermediate compound 71c (258 mg, 0.97 mmol, 74%) as yellow foam, used without further purification.

### d) Methyl (3aS,12bS)-11-fluoro-3-methylene-3,3a,8,12b-tetrahydrodibenzo[3,4:6,7]-cyclo-hepta[1,2-b]pyrrole-1 (2H)-carboxylate (intermediate compound 71d)

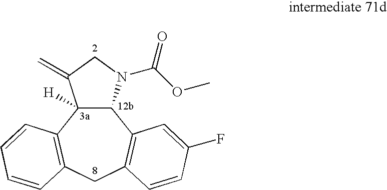

Reaction of intermediate compound 71c (258 mg, 0.97 mmol) was carried out in the same way as described for intermediate compound 9. Flash chromatography (Kieselgel 60, 230-400 mesh, heptane-EtOAc 50/50 to 0/100) afforded intermediate compound 71d (282 mg, 0.87 mmol, 90%) as yellow oil.

### e) Methyl (3aR,12bS)-11-fluoro-3-(hydroxymethyl)-3,3a,8,12b-tetrahydrodibenzo-[3,4:6,7]cyclohepta[1,2-b]pyrrole-1(2H)-carboxylate (intermediate compound 71e)

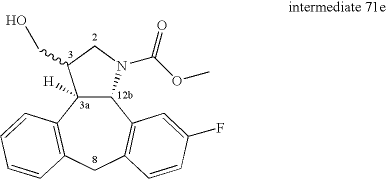

Reaction of 71d (255 mg, 0.79 mmol) was carried out obtained in the same way as described for intermediate compound 49. Flash chromatography (Kieselgel 60, 230-400 mesh, EtOH—CH₂Cl₂ 1/99 to 3/97) afforded 71e (215 mg, 0.63 mmol, 80%) as colorless oil.

### f) Methyl (3aR,12bS)-3-(azidomethyl)-11-fluoro-3,3a,8,12b-tetrahydrodibenzo-[3,4:6,7]cyclohepta[1,2-b]pyrrole-1(2H)-carboxylate (intermediate compound 71f)

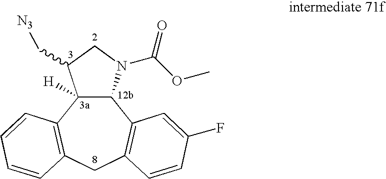

Reaction of intermediate compound 71f (215 mg, 0.63 mmol) was carried out obtained in the same way as described for intermediate compound 50. Flash chromatography (Kieselgel 60, 230-400 mesh, EtOAc) afforded intermediate compound 71f (194 mg, 0.53 mmol, 84%) as colorless oil.

### B. Preparation of the Final Compounds.

The compounds prepared hereinunder all are mixtures of isomeric forms, unless otherwise specified.

### EXAMPLE B1

### (4aS,13bR,14aS)-6-fluoro-2-methyl-1,2,3,4a,9,13b,14,14a-octahydrodibenzo-[3′,4′:6′,7′]cyclohepta[1′,2′:4,5]pyrrolo[1,2-c]imidazole (final compound 1)

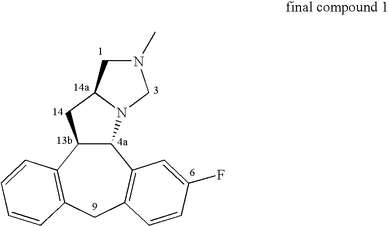

To a solution of intermediate compound 9 (130 mg, 0.3 mmol) in MeOH (5 mL) was added Et₃N (126.5 μL, 0.91 mmol) and the mixture was hydrogenated at 1 atmospheric pressure with 10% palladium-on-charcoal under vigorous stirring at room temperature. After 1 hour, formaldehyde (112.8 μL, 1.5 mmol) was added and the mixture was hydrogenated for an additional hour. The suspension was then filtered through a pad of celite and the solids were washed 4 times with CH₂Cl₂. After evaporation, the crude product was purified by column chromatography on silica gel using CHCl₃/MeOH (95/5). This yielded final compound 1 as an oil (50.5 mg, 54%).

Mass spectrum: -CI m/z (assignment, relative intensity) 309 (MH⁺, 100%), 289 (MH⁺—HF, 26%); EI: m/z (assignment, relative intensity) 308 (MH⁺, 68%), 279 (M⁺—CH₂NH, 4%), 265 (M⁺—CH₃NCH₂, 100%), 197 (23%); High resolution EI Calc lated C₂₀H₂₁FN₂ (M⁺): 308.1689, Found: 308.1684 (35%).

### EXAMPLE B2

### [(2S,3aR,12bS)-11-fluoro-1,2,3,3a,8,12b-hexahydrodibenzo[3,4:6,7]cyclohepta[1,2-b]-pyrrol-2-yl]-N,N-dimethylmethanamine (final compound 2)

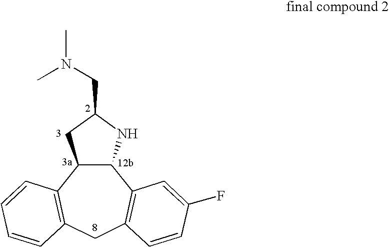

Dissolve final compound 1 (0.114 g, 0.37 mmol) in MeOH (10 mL) and add TFA (0.071 mL, 0.93 mmol), NaCNBH₃ (0.058 g, 0.93 mmol) and stir at room temperature for 1 hour. Add 10 mL K₂CO₃ (sat. aq. solution), extract with CH₂Cl₂ (3×10 mL) and dry with MgSO₄. Column purification on silica gel using CH₂Cl₂/MeOH (10%) gave final compound 2 as an oil (0.067 g, 59%).

Mass spectrum: -CI m/z (assignment, relative intensity) 311 (MH⁺, 100%), 291 (MH⁺—HF, 25%), 282 (MH⁺—CH₂NH), 266 (MH⁺—HN(CH₃)₂, 13%), 252 (8%); EI: m/z (assignment, relative intensity) 310 (M⁺, 26%), 266 (H⁺—(CH₃)₂N, 76%), 252 (M⁺—(CH₃)₂NCH₂, 70%), 235 (100%), 209 (61%); High resolution EI Calculated C₂₀H₂₃FN₂ (M⁺): 310.1845, Found: 310.1820 (5%).

### EXAMPLE B3

### (4aS,13bR,14aS)-6-fluoro-1,4a,9,13b,14,14a-hexahydrodibenzo-[3′,4′:6′,7′]cyclohepta-[1′,2′:4,5]pyrrolo[1,2-c]imidazole-3(2H)-thione (final compound 3)

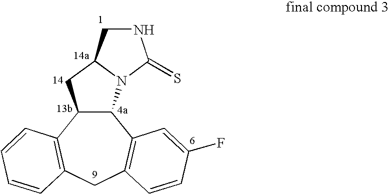

To solution of intermediate compound 9 (238.6 mg, 0.85 mmol) in DMF (3 mL) was added CS₂ (0.076 mL, 1.28 mmol). Stir at 60° C. for 20 minutes. After evaporation of the solvent, the residue was purified by column chromatography on silica gel using EtOAc/heptane (50/50) to give final compound 3 as a semisolid final compound (124.6 mg, 45%).

Mass spectrum: -CI m/z (assignment, relative intensity) 325 (MH⁺, 100%), 252 (1%), 224 (2%).

### EXAMPLE B4

### (5aS,14bR,15aS)-7-fluoro-2-methyl-1,2,3,5a,10,14b,15,15a-octahydro-4H-dibenzo [3′,4′:6′,7′]cyclohepta[1′,2′:4,5]pyrrolo[1,2-a]pyrazin-4-one (final compound 4)

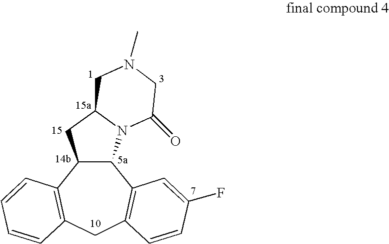

A solution of intermediate compound 16 (86.2 mg, 0.19 mmol) in MeOH (3 mL) was hydrogenated at 1 atmospheric pressure with 10% palladium-on-charcoal under vigorous stirring at room temperature. After reaction for 1 hour, formaldehyde (70.7 μL, 0.94 mmol) was added and the mixture was hydrogenated for an additional hour. The suspension was filtered through a pad of celite and the solids were washed 4 times with CH₂Cl₂. After evaporation of the solvent, the crude product was purified by column chromatograhy on silica gel using CHCl₃ to yield final compound 4 (18.6 mg, 29%). Mass spectrum: -CI m/z (assignment, relative intensity) 337 (MH⁺, 100%), 317 (MH⁺—HF, 18%), 309 (MH⁺—CO, 9%), 161 (9%), 133 (75%), 93 (72%).

### EXAMPLE B5

### [(2R,3aR,12bS)-11-fluoro-1-methyl-1,2,3,3a,8,12b-hexahydrodibenzo[3,4:6,7]cyclohepta[1,2-b]pyrrol-2-yl]-N,N-dimethylmethanamine (final compound 5)

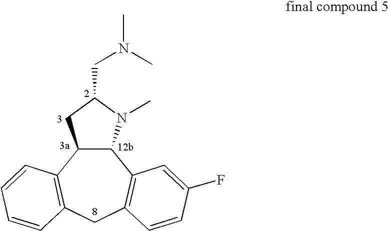

To a solution of intermediate compound 13 (28.7 mg, 0.05 mmol) in MeOH (2 mL) was added Et₃N (21.4 μL, 0.15 mmol) and formaldehyde (18.8 μL, 0.25 mmol) and the mixture was treated with hydrogen under 1 atmospheric pressure and 10% palladiumon-charcoal under vigorous stirring at room temperature. After reaction for 1 hour, the suspension was filtered through a pad of celite and the solids were washed 4 times with CH₂Cl₂. After evaporation, the crude product was purified by column chromatograhy on silica gel using CHCl₃/MeOH (90/10). This afforded final compound 5 as an oil (16.7 mg, 98%).

Mass spectrum: -CI m/z (assignment, relative intensity) 325 (MH⁺, 100%), 323 (25%), 305 (MH⁺—HF, 19%), 280 (MH⁺—HN(CH₃)₂, 12%), 266 (MH⁺—CH₃N(CH₃)₂, 36%).

### EXAMPLE B6

### (4aS,13bR,14aR)-6-fluoro-1,4a,9,13b,14,14a-hexahydrodibenzo[3′,4′:6′,7′]cyclohepta-[1′,2′:4,5]pyrrolo[1,2-c]imidazol-3(2H)-one (final compound 6)

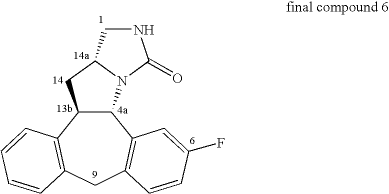

To a solution of intermediate compound 13 (40.4 mg, 0.14 mmol) in CH₃CN (2 mL) was added Et₃N (50 μL, 0.36 mmol) and the mixture was heated under argon at 70° C. After 1 hour, a solution of diphenyl carbonate (36.6 mg, 0.17 mmol) in CH₃CN was added dropwise and the mixture was stirred at 70° C. for 2 days. After evaporation, the crude product was purified by column chromatography on silica gel using EtOAc/heptane (20/80) to yield final compound 6 as an oil (23 mg, 52%).

Mass spectrum: -CI m/z (assignment, relative intensity) 309 (MH⁺, 100%), 308 (12%), 289 (MH⁺—HF, 20%), 279 (3%), 113 (8%).

### EXAMPLE B7

### (4aS,13bR,14aR)-6-fluoro-2-methyl-1,4a,9,13b,14,14a-hexahydrodibenzo[3′,4′:6′,7′]-cyclohepta 1,2′:4,5]pyrrolo[1,2-c]imidazol-3(2H)-one (final compound 7)

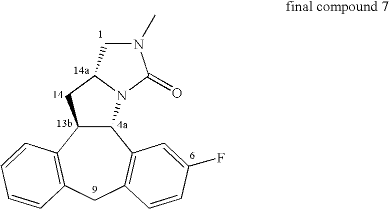

To a solution of final compound 6 (29 mg, 0.10 mmol) in THF (3 mL) was added NaH (15.9 mg, 0.31 mmol) and the mixture was stirred at room temperature for 20 minutes. Then Me₂SO₄ (25.4 mg, 0.26 mmol) was added and the mixture was stirred for an additional 30 minutes. Add 10 mL of NH₄Cl (sat. aq. solution), extract with CH₂Cl₂ (3×10 mL) and dry with MgSO₄. Column purification on silica gel using EtOAc/heptane (40/60) gave the final compound 7 as an oil (19 mg, 63%).

Mass spectrum: -CI m/z (assignment, relative intensity) 323 (MH⁺, 100%), 303 (MH⁺—HF, 26%), 209 (2%), 127 (3%).

### EXAMPLE B8

### (4aS,13bR,14aR)-6-fluoro-1,4a,9,13b,14,14a-hexahydrodibenzo[3 ′,4′:6′,7′]cyclohepta-[1′,2′:4,5]pyrrolo[1,2-c]imidazole-3(2H)-thione (final compound 8)

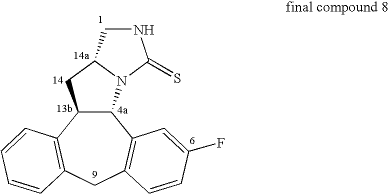

To a solution of intermediate compound 13 (54 mg, 0.19 mmol) in DMF (3 mL) was added CS₂ (17.3 μL, 0.29 mmol). After stirring at 60° C. for 20 minutes, followed by evaporation of the solvent, column chromatography purification on silica gel (eluent: EtOAc/heptane (50/50)) gave a crystalline final compound 8 (27.3 mg, 44%); mp: 150-151° C.

Mass spectrum: -CI m/z (assignment, relative intensity) 325 (MH⁺, 100%), 252 (1%), 224 (2%).

### EXAMPLE B9

### (4aS,13bR,14aR)-6-fluoro-3-(methylsulfanyl)-1,4a,9,13b,14,14a-hexahydrodibenzo-[3′,4′:6′,7′]cyclohepta[1′,2′:4,5]pyrrolo[1,2-c]imidazole (final compound 9)

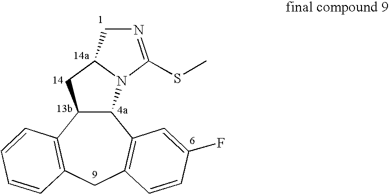

To a solution of final compound 8 (140.6 mg, 0.43 mmol) in MeOH (10 mL) was added methyl iodide (53.5 μL, 0.86 mmol) and Et₃N (129 μl, 0.86 mmol). After stirring at 80° C. for 2 days, solvent and reagents were evaporated. Column purification on silica gel (eluent: EtOAc/heptane (40/60)) gave the final compound 9 as an oil (72.7 mg, 49%).

Mass spectrum: -CI m/z (assignment, relative intensity) 339 (MH⁺, 100%), 319 (MH⁺—HF, 4%), 268 (8%), 266 (3%).

### EXAMPLE B10

### [(2R,3aR,12bS)-11-fluoro-1-(methoxyacetyl)-1,2,3,3a,8,12b-hexahydrodibenzo[3 ,4:6,7]cyclohepta[1,2-b]pyrrol-2-yl]-N,N-dimethylmethanamine (final compound 10)

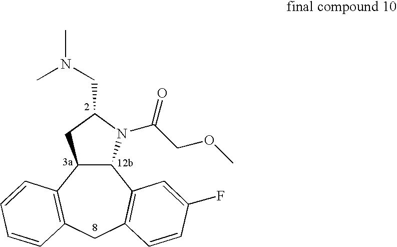

To a solution of intermediate compound 19 (265.3 mg, 0.44 mmol) in MeOH (20 mL) was added MeSO₃H (2 mL) and the mixture was stirred at 60° C. for 30 minutes. After evaporation of the solvent, NaHCO₃ (sat. aq. solution) (15 mL) was added and the mixture was extracted with CH₂Cl₂ (3×10 mL). The combined organic layers were dried with MgSO₄. Column chromatography purification on silica gel using CH₂Cl₂/MeOH (5%) gave the amino compound (125 mg, 79%). The latter was then dissolved in MeOH (30 mL). Following addition of formaldehyde (80 μL, 1.06 mmol) the mixture was hydrogenated (1 atmospheric pressure) with 10% palladium-on-charcoal under vigorous stirring at room temperature for 6 hours. The suspension was then filtered through a pad of celite and the solids were washed 4 times with CH₂Cl₂. After evaporation of the solvent, the crude product was purified by column chromatograhy on silica gel using CHCl₃/MeOH (95/5). Final compound 10 (90.1 mg, 67%) was obtained as an oil (mixture of conformers).

Mass spectrum: -APCI m/z (assignment, relative intensity) 383 (MH⁺, 100%), 369 (4%), 367 (4%), 363 (MH⁺—HF, 5%), 354 (2%), 351 (2%).

### EXAMPLE B11

### Methyl ({[(2R,3aR,12bS)-11-fluoro-1,2,3,3a,8,12b-hexahydrodibenzo[3,4:6,7]cyclo-hepta[1,2-b]pyrrol-2-yl]methyl}amino)acetate (final compound 11)

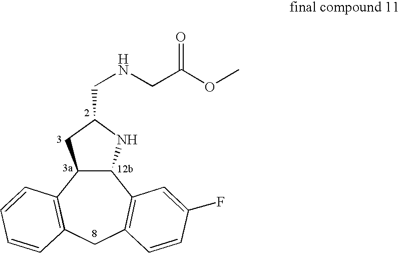

Intermediate compound 21 (53.4 mg, 0.15 mmol) was dissolved in a sat. solution of HCl in MeOH (10 mL) and the mixture was stirred at 60° C. overnight. After removal of solvent, 10 mL of K₂CO₃ (sat. aq. solution) was added and the mixture extracted with CH₂Cl₂ (3×10mL). Column chromatography purification on silica gel using CHCl₃/MeOH (97/3) as eluent gave the final compound 11 (20 mg, 37%) as an oil.

Mass spectrum: -CI m/z (assignment, relative intensity) 355 (MH⁺, 100%), 335 (MH⁺—HF, 14%), 295 (MH⁺—CH₃OH—CO, 4%), 252 (MH⁺—CH₃OH—CH₂CO—NHCH₂, 8%), 169 (5%), 141 (46%); EI m/z (assignment, relative intensity) 354 (M⁺, 3%), 295 (M⁺—CH₃OCO, 4%), 252 (M⁺—CH₃OCOCH₂NHCH₂, 100%), 235 (M⁺—CH₃OCOCH₂NHCH₂—NH₃, 68%), 223 (8%), 209 (22%); High resolution EI Calculated C₂₁H₂₃N₂O₂F (M⁺): 354.1744,Found: 354.1751 (9%).

### EXAMPLE B12

### (5aS,14bR,15aR)-7-fluoro-1,2,3,5a,10,14b,15,15a-octahydro-4H-dibenzo[3′,4′:6′,7′]-cyclohepta[1′,2′:4,5]pyrrolo[1,2-a]pyrazin-4-one (final compound 12)

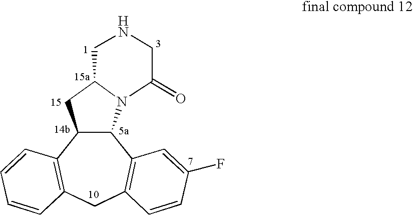

Intermediate compound 21 (250 mg, 0.71 mmol) was dissolved in 10 mL of HCl in MeOH (sat. solution) and the mixture was stirred at room temperature overnight. The reaction was quenched by addition of 10 mL of K₂CO₃ (sat. aq. solution). The mixture was then extracted 3 times with 10 mL CH₂Cl₂. The combined organic layers were dried over MgSO₄ and evaporated. Column chromatography purification on silica gel using CHCl₃/MeOH (95/5) as eluent gave final compound 12 (67.6 mg, 29%) as an oil.

Mass spectrum: -CI m/z (assignment, relative intensity) 323 (MH⁺, 100%), 303 (MH⁺—HF, 20%), 295 (MH⁺—CO, 2%), 252 (MH⁺—COCH₂—NHCH₂, 1%), 188 (2%), 160 (5%); EI m/z (assignment, relative intensity) 322 (M⁺, 100%), 252 (M⁺—COCH₂N═CH2, 40%), 235 (68%), 223 (M⁺—COCH₂N═CH₂—CH₂NH, 44%), 207 (13%), 209 (88%), 209 (22%); High resolution El Calculated C₂₀H₁₉N₂OF (M⁺): 322.1481, Found: 322.1484 (100%).

### EXAMPLE B13

### (5aS,14bR,15aR)-7-fluoro-2-methyl-1,2,3,5a,10,14b,15,15a-octahydro-4H-dibenzo-[3′,4′:6′,7′]cyclohepta[1′,2′:4,5]pyrrolo[1,2-a]pyrazin-4-one (final compound 13)

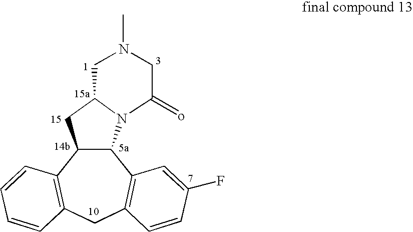

To a solution of final compound 12 (82.3 mg, 0.25 mmol) in MeOH (10 mL) was added formaldehyde (96 μL, 1.22 mmol) and the mixture was hydrogenated (1 atmospheric pressure) with 10% palladium-on-charcoal under vigorous stirring at room temperature for 1 hour. Then the mixture was filtered through a pad of celite and the solids were washed 4 times with CH₂Cl₂. After evaporation, the crude product was purified by column chromatograhy on silica gel using CHCl₃/MeOH (3%) as eluent. Final compound 13 (43.4 mg, 50%) was obtained as a solid; mp: 139-141° C.

Mass spectrum: -CI m/z (assignment, relative intensity) 337 (MH⁺, 100%), 317 (MH⁺—HF, 30%), 279 (1%), 251 (1%), 209 (1%); EI m/z (assignment, relative intensity) 336 (M⁺, 74%), 293 (M⁺—COCH₃, 13%), 265 (M⁺—CO═CHNHCH₃, 9%), 233 (18%), 209 (42%), 196 (26%), 57 (100%); High resolution EICalculated C₂₁H₂₁N₂OF (M⁺): 336.1638, Found: 336.1641 (100%).

### EXAMPLE B14

### (5aS,14bR,15a,R)-7-fluoro-2-methyl-1,3,4,5a,10,14b,15,1 5a-octahydro-2H-dibenzo-[3′,4′:6′,7′]cyclohepta[1′,2′:4,5]pyrrolo[1,2-a]pyrazine (final compound 14)

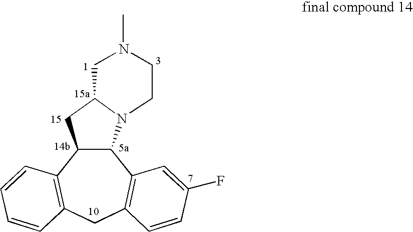

To a solution of final compound 13 (34.3 mg, 0.1 mmol) in THF (10 mL) was added BH₃.Me₂S (100 μL, 0.2 mmol) and the mixture was heated at 85° C. overnight. Following evaporation of the solvent the residue was dissolved in 10 mL of HCl in MeOH (sat. solution) and the mixture was refluxed for 30 minutes. After removal of the solvent 10 mL of K₂CO₃ (sat. aq. solution) was added and the solution extracted 4 times with CH₂Cl₂. Then, the combined organic layers were evaporated and the crude product was purified by column chromatograhy on silica gel using CHCl₃/MeOH (3%) as eluent. Final compound 14 (15.9 mg, 50%) was obtained as an oil.

Mass spectrum: -CI m/z (assignment, relative intensity)323 (MH⁺, 73%), 303 (MH⁺—HF, 18%), 247 (4%), 219 (3%), 43 (100%o); EI m/z (assignment, relative intensity) 322 (M⁺, 73%), 278 (M⁺—N(CH₃)₂, 44%), 266 (M⁺—N(CH₂)₃, 85%), 264 (M⁺—CH₂CH₂NHCH₃, 94%), 251 (M⁺—CH₂CH₂—CH₂N(CH₃), 100%), 209 (68%), 196 (38%); High resolution EI Calculated C₂₁H₂₃N₂F (M⁺): 322.1845, Found: 322.1849 (100%).

### EXAMPLE B 15

### (4aS,13bR,14aS)-6-fluoro-1,4a,9,13b,14,14a-hexahydrodibenzo[3′,4′:6′,7′]cyclohepta-[1′,2′:4,5]pyrrolo[1,2-c]imidazol-3(2H)-one (final compound 15)

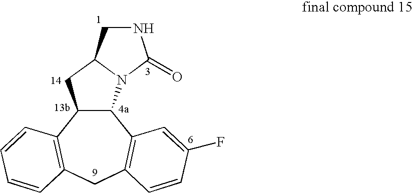

To the intermediate compound 25 (13.5 mg, 0.04 mmol) in CH₂Cl₂ (1 mL) was added CH₃SO₃H (1.3 μL, 0.02 mmol). After stirring at room temperature for 1 minute, the mixture was worked up by adding Na₂CO₃ (sat. aq. sol.). Extract 3 times with CH₂Cl₂ and dry with MgSO₄. Column chromatography purification on silica gel using CHCl₃/MeOH (95/05) gave final compound 15 as an oily product (10.5 mg, 82%). Mass spectrum: -CI m/z (assignment, relative intensity) 309 (MH⁺, 100 91%), 289 (MH⁺—HF, 17%), 257 (1%).

### EXAMPLE B 16

### (4aS,13bR,14aS)-6-fluoro-2-methyl-1,4a,9,13b,14,14a-hexahydrodibenzo[3′,4′:6′,7′]-cyclohepta[1′,2′:4,5]pyrrolo[1,2-c]imidazol-3(2H)-one (final compound 16)

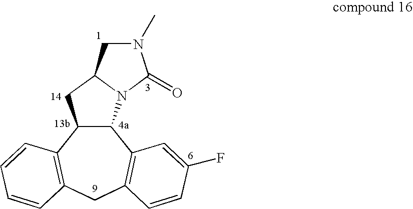

To a solution of final compound 15 (10 mg, 0.03 mmol) in THF (1 mL) was added NaH (5 mg, 0.1 mmol) and the mixture was stirred at room temperature for 20 minutes. Then Me₂SO₄ (8 μL, 0.08 mmol) was added and the mixture was stirred for additional 30 min. Add 10 mL of NH₄Cl (sat. aq. solution), extract with CH₂Cl₂ (3×10 mL) and dry with MgSO₄. Column chromatrography purification on silica gel using EtOAc/heptane (50/50) as eluent gave the final compound 16 (8.4 mg, 80%) as an oil. Mass spectrum: -CI m/z (assignment, relative intensity) 323 (MH⁺, 100%), 303 (MH⁺—HF, 6%), 257 (11%), 252 (MH⁺—CH₂N(CH₃)CO, 9%), 229 (9%).

### EXAMPLE B 17

### [(2R,3aR,12bS)-11-fluoro-2,3,3a,12b-tetrahydro-lH-dibenzo[2,3 :6,7]oxepino[4,5-b]pyrrol-2-yl]-N,N-dimethylmethanamine (final compound 17)

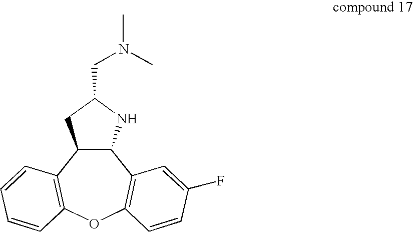

To a solution of intermediate compound 38 (0.17 g, 0.5 mmol), CH₂O (3 eq.) and AcOH (3 eq.) in MeOH (5 mL) at 0° C., NaCNBH₃ (4 eq.) was added in several lots. The reaction mixture was warmed to room temperature and stirred for 6 hours. Solid NaHCO₃ (0.5 g) was added to the reaction mixture and stirred for 0.5 hour. To remove inorganic complexes the reaction mixture was put on sort filtration column and diluted with CH₂Cl₂:MeOH (9.5:0.5). The crude intermediate compound

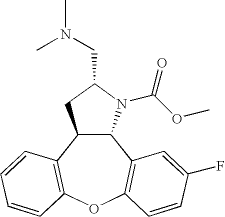

thus obtained was dissolved in i-PrOH (4 mL) and a solution of KOH (56 mg) in water (0.5 mL) was added to it and then refluxed for 3 hours. Silica gel was added to the reaction mixture and the solvent was removed under reduced pressure followed by purification of compound by flash column chromatography using CH₂Cl₂:MeOH (9:1) as an eluent to obtain final compound 17 as a thick viscous liquid (60%, 93 mg).

HRMS. Calculated 312.1638; found 312.1633.

### EXAMPLES B18-20

a) To a solution of intermediate compound 39 (0.5 mmol, 0.33 g) in dioxane (5 mL) the corresponding amino alcohol (5 eq.) was added and then refluxed for 6 hours. The solvent was removed under reduced pressure followed by column chromatography (silica gel) using CH₂Cl₂:MeOH (9:1) as an eluent to obtain intermediate compounds 39a, 39b and 39c as a thick viscous liquids in 40-50% overall yield.

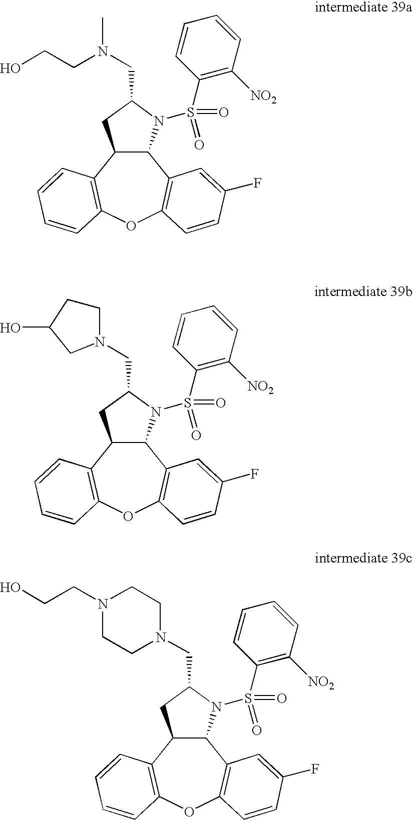

b) A mixture of appropriate intermediate compounds 39a, 39b and 39c (ca. 0.4 mmol), thiophenol (110 mg, 1.0 mmol), anhydrous K₂CO₃ (138 mg, 1 mmol) and DMF (20 mL) was stirred at 80° C. for 4 hours, cooled to ambient temperature, diluted with water, product extracted with EtOAc (3×50 mL), combined organics washed with water (4×50 mL), brine (35 mL), dried (K₂CO₃), evaporated and purified by solid phase extraction on basic alumina (Brockmann II, heptane-ethyl acetate 50/50, then ethyl acetate-MeOH 100/0 to 96/4 to 90/10) to obtain final compounds 18, 19 and 20.

### 2-[{[(2R,3aR,12bS)-11-fluoro-2,3,3a,12b-tetrahydro-1H-dibenzo[2,3 :6,7]oxepino[4,5-b]pyrrol-2-yl]methyl}(methyl)amino]ethanol (final compound 18)

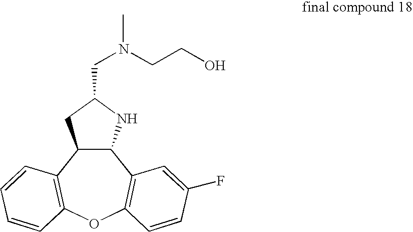

HRMS: Calculated 342.1744; found 342.1750

### 2-(4-{[(2R,3aR,12bS)-11-fluoro-2,3,3a,12b-tetrahydro-1H-dibenzo[2,3 :6,7]oxepino-[4,5-b]pyrrol-2-yl]methyl}-1-piperazinyl)ethanol (final compound 19)

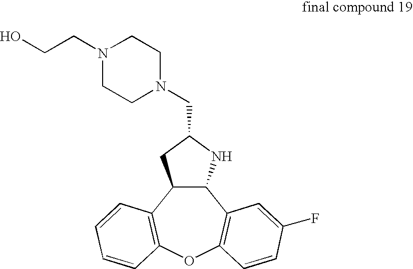

HRMS: Calculated 397.2166, found 397.2158

### 1-{[(2R,3aR,12bS)-11-fluoro-2,3,3a,12b-tetrahydro-1H-dibenzo[2,3:6,7]oxepino[4,5-b]pyrrol-2-yl]methyl}-3-pyrrolidinol (final compound 20)

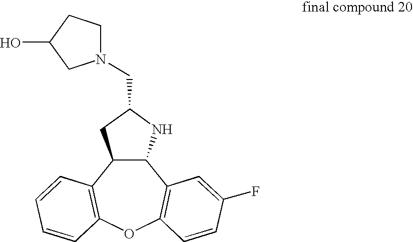

HRMS: Calculated 354.1744; found 354.1755

### EXAMPLE B21

### [(2S,3aR,12bS)-11-fluoro-3,3a,8,12b-tetrahydro-2H-dibenzo[3,4:6,7]cyclohepta[1,2-b]thien-2-yl]-N,N-dimethylmethanamine (final compound 21)

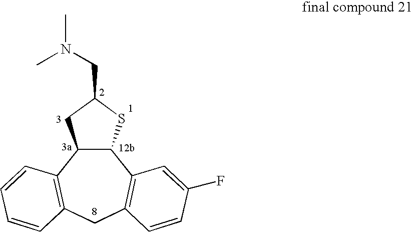

To a solution of intermediate compound 43 (81 mg, 0.25 mmol) in THF and water (3 mL/1 mL) was added PPh₃ (0.13 g, 0.50 mmol). The reaction mixture was stirred at room temperature for 1 night. After evaporation of the solvent, MeOH (5 mL), HCHO (37 wt % aq. solution, 0.20 mL, 2.5 mmol), AcOH (1 mL) and NaCNBH₃ (75 mg, 1.20 mmol) were added. Stirring was continued at room temperature for 1 day. Add Na₂CO₃ (sat. aq. sol.), extract 3 times with CH₂Cl₂. Column chromatography purification on silica gel using EtOAc as eluent gave final compound 21 as an oily product (70 mg, 86%).

Mass spectrum: -CI m/z (assignment, relative intensity) 328 (MH⁺, 100%), 308 (MH⁺—HF, 20%), 283 (M^{H}-Me₂NH, 40%), 249 (MH⁺-Me₂NH—H₂S, 12%).

### EXAMPLE B22

### [(2S,3aR,12bS)-11-fluoro-1,1-dioxido-3,3a,8, 12b-tetrahydro-2H-dibenzo[3,4:6,7]-cyclohepta[1,2-b]thien-2-yl]-N,N-dimethylmethanamine (final compound 22)

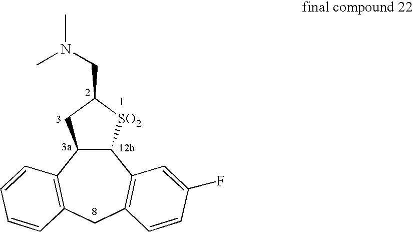

To a solution of intermediate compound 44 (133.5 mg, 0.37 mmol) in THF (8 mL) was added water (67.0 μL, 3.74 mmol) and PPh₃ (0.13 g, 0.50 mmol). The reaction mixture was stirred at room temperature for 1 night. After evaporation of the solvent, 5 mL of MeOH, HCHO (37 wt % aq. solution, 0.24 mL, 2.98 mmol), AcOH (0.5 mL) and NaCNBH₃ (94 mg, 1.49 mmol) were added. Stirring was continued at room temperature for 1 day. Add Na₂CO₃ (sat. aq. sol.), extract 3 times with CH₂Cl₂. Column chromatography purification on silica gel using CH₂Cl₂/MeOH (95/05) as eluent gave final compound 22 as an oil product (60.7 mg, 45%).

Mass spectrum: -CI m/z (assignment, relative intensity) 360 (MH⁺, 100%), 358 (6%), 340 (MH⁺—HF, 12%), 303 (8%), 294 (MH⁺—H₂SO₂, 4%), 250 (1%).

### EXAMPLE B23

### [(2R,3aR,12bS)-11-fluoro-3,3a,8,12b-tetrahydro-2H-dibenzo[3,4:6,7]cyclohepta[1,2-b]thien-2-yl]-N,N-dimethylmethanamine (final compound 23)

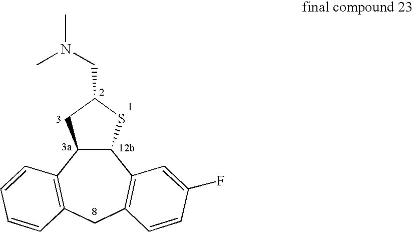

To a solution of intermediate compound 47 (0.15 g, 0.46 mmol) in THF and water (5 mL/1 mL) was added PPh₃ (0.13 g, 0.50 mmol). After stirring at room temperature for 1 night and evaporation of the solvent, 5 mL of MeOH, HCHO (37 wt % aq. solution, 0.20 mL, 2.5 mmol), AcOH (1 mL) and NaCNBH₃ (75 mg, 1.20 mmol) were added. Stirring was continued at room temperature for 1 day. Add Na₂CO₃ (sat. aq. sol.), extract 3 times with CH₂Cl₂. Column chromatography purification on silica gel using EtOAc as eluent gave final compound 23 as an oily product (70 mg, 86%). Mass spectrum: -CI m/z (assignment, relative intensity) 328 (MH⁺, 100%), 308 (MH⁺—HF, 20%), 283 (MH⁺-Me₂NH, 40%), 249 (MH⁺-Me₂NH—H₂S, 12%).

### EXAMPLE B24

### [(2R,3aR,12bS)-11-fluoro-1,1-dioxido-3,3a,8,12b-tetrahydro-2H-dibenzo[3,4:6,7]-cyclohepta[1,2-b]thien-2-yl]-N,N-dimethylmethanamine (final compound 24)

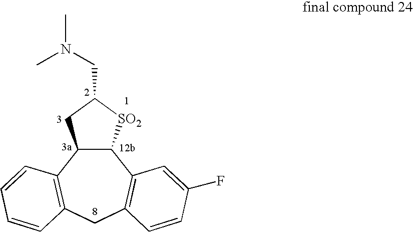

To a solution of intermediate compound 48 (146.5 mg, 0.41 mmol) in THF (8 mL) was added water (74.0 mL, 4.10 mmol) and PPh₃ (0.215 mg, 0.82 mmol). The reaction mixture was stirred at room temperature for 1 night. After evaporation of the solvent, 5 mL of MeOH, HCHO (37 wt % aq. solution, 0.28 mL, 3.51 mmol), AcOH (0.5 mL) and NaCNBH₃ (110.0 mg, 1.75 mmol) were added. Stirring was continued at room temperature for 1 day. Add Na₂CO₃ (sat. aq. sol.), extract 3 times with CH₂Cl₂. Column chromatography purification on silica gel using CH₂Cl₂/MeOH (90/10) gave final compound 24 as an oily product (105.0 mg, 71%).

Mass spectrum: -CI m/z (assignment, relative intensity) 360 (MH⁺, 100%), 358 (6%), 340 (MH⁺—HF, 12%), 303 (8%), 294 (MH⁺—H₂SO₂, 4%), 250 (1%).

### EXAMPLE B25

### [(2S,3aR,12bS)-11-fluoro-1-oxido-3,3a,8,12b-tetrahydro-2H-dibenzo[3,4:6,7]cyclohepta[1,2-b]thien-2-yl]-N,N-dimethylmethanamine (final compound 25)

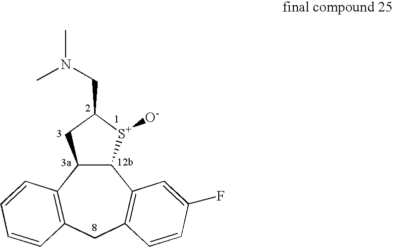

To a solution of intermediate compound 49 (107.9 mg, 0.32 mmol) in THF (5 mL) was added water (57 μL, 3.16 mmol) and PPh₃ (166.0 mg, 0.63 mmol). The reaction mixture was stirred at room temperature for 1 night. After evaporation of the solvent 5 mL of MeOH, HCHO (37%, 0.26 mL, 3.33 mmol), AcOH (0.5 mL) and NaCNBH₃ (104.7 mg, 1.67 mmol) were added. Stirring was continued at room temperature for 1 day. Add Na₂CO₃ (sat. aq. sol.), extract 3 times with CH₂Cl₂. Column chromatography purification on silica gel using CH₂Cl₂/MeOH (95/05) as eluent gave final compound 25 as an oily product (80.4 mg, 74%).

Mass spectrum: -CI m/z (assignment, relative intensity) 344 (MH⁺, 100%), 328 (MH⁺—O, 13%), 326 (MH⁺—H₂O, 15%), 324 (MH⁺—HF, 15%), 182 (14%), 100 (27%).

### EXAMPLE B26

### [(2S,3aR,12bS)-11-fluoro-1-oxido-3,3a,8,12b-tetrahydro-2H-dibenzo[3,4:6,7]-cyclohepta[1,2-b]thien-2-yl]-N,N-dimethylmethanamine (final compound 26)

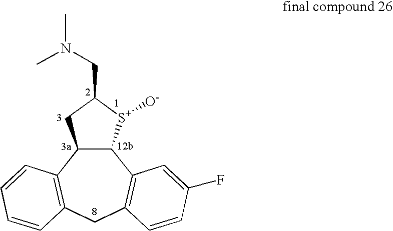

To a solution of intermediate compound 50 (133.4 mg, 0.39 mmol) in THF (5 mL) was added water (70 μL, 3.91 mmol) and PPh₃ (205.2 mg, 0.78 mmol). The reaction mixture was stirred at room temperature for 1 night. After evaporation of the solvent, 5 mL of MeOH, HCHO (37%, 0.24 mL, 2.99 mmol), AcOH (0.4 mL) and NaCNBH₃ (94.0 mg, 1.50 mmol) were added. Stirring was continued at room temperature for 1 day. Add Na₂CO₃ (sat. aq. sol.), extract 3 times with CH₂Cl₂. Column chromatography purification on silica gel using CH₂Cl₂/MeOH (95/05) as eluent gave final compound 26 as an oily product (85.2 mg, 63%).

Mass spectrum: -CI m/z (assignment, relative intensity) 344 (MH⁺, 100%), 328 (MH⁺—O, 10%), 327 (12%), 326 (M⁺—H₂O, 46%), 324 (MH⁺—HF, 22%), 283 (12%).

### EXAMPLE B27

### [(2R,3aR,12bS)-11-fluoro-1-oxido-3,3a,8,12b-tetrahydro-2H-dibenzo[3,4:6,7]cyclohepta[1,2-b]thien-2-yl]-N,N-dimethylmethanamine (final compound 27)

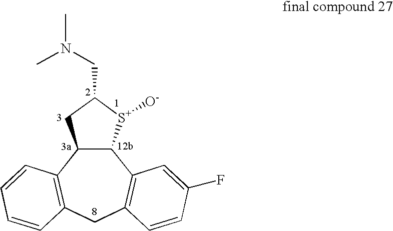

To a solution of intermediate compound 51 (85 mg, 0.25 mmol) in THF (5 mL) was added water (45 μL, 2.49 mmol) and PPh₃ (130.8 mg, 0.50 mmol). The reaction mixture was stirred at room temperature for 1 night. After evaporation of the solvent, 5 mL of MeOH, HCHO (37%, 0.08 mL, 1.03 mmol), AcOH (0.3 mL) and NaCNBH₃ (32 mg, 0.52 mmol) were added. Stirring was continued at room temperature for 1 day. Add Na₂CO₃ (sat. aq. sol.), extract 3 times with CH₂Cl₂. Column chromatography purification on silica gel using CH₂Cl₂/MeOH (95/05) as eluent gave final compound 27 as an oily product (35 mg, 41%).

Mass spectrum: -CI m/z (assignment, relative intensity) 344 (MH⁺, 100%), 328 (MH⁺—O, 4%), 327 (3%), 326 (MH⁺—H₂O, 10%), 324 (MH⁺—HF, 8%), 281 (6%).

### EXAMPLE B28

### [(2R,3aR,12bS)-11-fluoro-1-oxido-3,3a,8,12b-tetrahydro-2H-dibenzo[3,4:6,7]cyclohepta[1,2-b]thien-2-yl]-N,N-dimethylmethanamine (final compound 28)

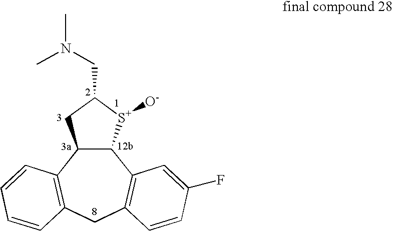

To a solution of intermediate compound 52 (158.5 mg, 0.46 mmol) in THF (5 mL) was added water (84 μL, 4.65 mmol) and PPh₃ (243.8 mg, 0.93 mmol). The reaction mixture was stirred at room temperature for 1 night. After evaporation of the solvent, 5 mL of MeOH, HCHO (37%, 0.32 mL, 4.05 mmol), AcOH (0.5 mL) and NaCNBH₃ (130 mg, 2.03 mmol) were added. Stirring was continued at room temperature for 1 day. Add Na₂CO₃ (sat. aq. sol.), extract 3 times with CH₂Cl₂. Column chromatography purification on silica gel using CH₂Cl₂/MeOH (95/05) as eluent gave final compound 28 as an oily product (115.7 mg, 72%).

Mass spectrum: -CI m/z (assignment, relative intensity) 344 (MH⁺, 100%), 328 (MH⁺—O, 3%), 327 (3%), 326 (MH⁺—H₂O, 13%), 324 (MH⁺—HF, 14%), 281 (6%).

### EXAMPLE B29

### (3R,4aR,13bR)-12-fluoro-2,3,4,4a,9,13b-hexahydrodibenzo[3,4:6,7]cyclohepta[1,2-b]-pyran-3-ol (final compound 29)

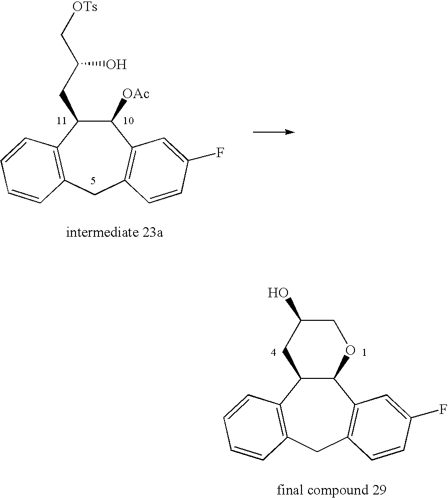

Dissolve intermediate compound 23a (1.31 g, 2.63 mmol) in CH₂Cl₂ (50 mL). Add DHP (1.20 mL, 13.2 mmol) and CSA (6 mg, 0.026 mmol). Stir at room temperature for 5 hours. Evaporate the solvent and dissolve the residue in 50 mL MeOH. Add K₂CO₃ (0.73 g, 5.26 mmol) and stir at room temperature for 1 night. Work up by adding sat. aq. NH₄Cl (30 mL), extract with CH₂Cl₂ (3×15 mL) and dry with MgSO₄. Evaporate the solvent and dissolve the residue in dry THF (50 mL). Add NaH (0.24 g, 7.78 mmol) and stir at room temperature for 1 day. Add 30 mL sat. aq. NH₄Cl, extract with CH₂Cl₂ (3×20 mL) and dry the organic phases with MgSO₄. Column purification on silica gel using Et₂O/hexane (35/65) gave an oil (0.86 g, 90% from 2). Dissolve this oil (0.86 g, 2.34 mmol) in 20 mL MeOH/H₂O (9/1) and add Dowex 50WX8-100 (1.00 g). Heat the mixture at 50° C. for 1 night. Filter through a P3 filter, wash the solids with CH₂Cl₂ (5×15 mL) and evaporate the solvent. Column chromatography purification on silica gel using ether/hexane (70:30) as eluent yielded final compound 29 as an oil (0.61 g, 93%).

Mass spectrum: -CI m/z (assignment, relative intensity) 285 (MH⁺, 25%), 267 (MH⁺—H₂O, 100%), 249 (MH⁺-2 H₂O, 36%); EI: m/z (assignment, relative intensity) 284 (M⁺, 1%), 209 (M⁺-CH₂CHOHCH₂OH, 100%); High resolution EI Calculated C₁₈H₁₇FO₂ (M⁺): 284.1213, Found: 284.1204 (2%).

### EXAMPLE B30

### a) (3R,4aR,13bR)-12-fluoro-2,3,4,4a,9,13b-hexahydrodibenzo[3,4:6,7]cyclohepta [1,2-b]pyran-3-yl methanesulfonate (intermediate compound 53)

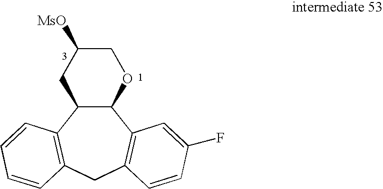

Dissolve final compound 29 (0.61 g, 2.16 mmol) in CH₂Cl₂ (50 mL). Add Et₃N (0.60 mL, 4.32 mmol), DMAP (0.13 g, 1.08 mmol) and MsCl (0.25 mL, 3.24 mmol). Stir at room temperature for 4 hours. Work up by adding sat. aq. NH₄Cl (20 mL), extract with CH₂Cl₂ (3×20 mL) and dry with MgSO₄. Column chromatography purification on silica gel using CH₂Cl₂ as eluent yielded intermediate compound 53 as an oil (0.76 g, 97%).

Mass spectrum: -CI m/z (assignment, relative intensity) 363 (MH⁺, 1%), 267 (MH⁺-MsOH, 100%), 249 (MH⁺-MsOH—H₂O, 33%); EI: m/z (assignment, relative intensity) 362 (M⁺, 5%), 266 (M⁺-MsOH, 3%), 248 (M⁺-MsOH—H₂O, 4%), 209 (M⁺—CH₂CHOMsCH₂OH, 100%); High resolution EI Calculated Cl₉H₁₉FO₄S (M⁺): 362.0988, Found: 362.0984 (12%).

### b) (3S,4aR,13bR)-3-azido-12-fluoro-2,3,4,4a,9,13b-hexahydrodibenzo[3,4:6,7]bcyclohepta[1,2-b]pyran (intermediate compound 54)

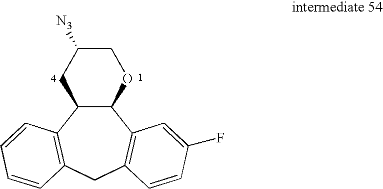

Dissolve intermediate compound 53 (0.29 g, 0.79 mmol) in DMF (10 mL), add NaN₃ (0.10 g, 1.58 mmol) and heat the mixture at 90° C. for 2 hours. Add sat. aq. NH₄Cl (10 mL), extract with CH₂Cl₂ (3×10 mL) and dry with MgSO₄. Column chromatography purification on silica gel using CH₂Cl₂/heptane (40:60) as eluent yielded intermediate compound 54 as a crystalline product (0.22 g, 88%); mp: 91-93° C.

Mass spectrum: -CI m/z (assignment, relative intensity) 310 (M^{H+}, 13%), 282 (MH⁺—N₂, 100%); EI. m/z (assignment, relative intensity) 281 (M^{+- N}₂, 28%), 208 (100%): High resolution EI Calculated C₁₈H₁₆FNO (MH⁺—N₂): 309.1216, Found: 309.1223 (40%).

### c) (3S,4aR,13bR)-12-fluoro-2,3,4,4a,9,13b-hexahydrodibenzo[3,4:6,7]cyclohepta[1,2-b]pyran-3-amine (final compound 30)

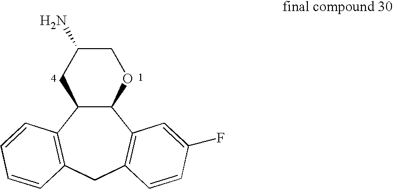

Dissolve intermediate compound 54 (0.16 g, 0.52 mmol) in i-PrOH/THF (2:1, 15 mL). Add 10% Pd—C (ca. 100 mg) and subject to hydrogenation (1 atmospheric pressure) for 1 night. Filter through a pad of celite, wash the solids with CH₂Cl₂ (5×10 mL) and evaporate the filtrate. The residue is purified by column chromatography on silica gel using CHCl₃/MeOH (75:25) as eluent to give final compound 30 as a crystalline product (0.14 g, 94%); mp:74-76° C.

Mass spectrum: -CI m/z (assignment, relative intensity) 284 (MH⁺, 100%); EI: m/z (assignment, relative intensity) 283 (M⁺, 5%), 209 (M⁺—CH₂CHNH₂CH₂OH, 100%); High resolution EI Calculated C₁₈H₁₈FNO (Me): 283.1372, Found: 283.1370 (43%).

### EXAMPLE B31

### (4aR,13bR)-12-fluoro-4,4a,9,13b-tetrahydrodibenzo[3,4:6,7]cyclohepta[1,2-b]pyran-3(2H)-one (final compound 31)

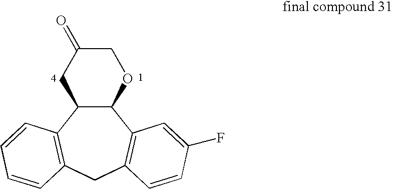

Dissolve final compound 29 (77 mg, 0.27 mmol) in CH₂Cl₂ (10 mL) and add PCC (131 mg, 0.54 mmol). Stir at room temperature for 20 hours. Filter through a pad of celite, wash the solids with CH₂Cl₂ (5×20 mL) and evaporate the filtrates. Column chromatography purification on silica gel using Et₂O/hexane (50:50) as eluent yielded final compound 31 as a white crystalline product (61 mg, 80%); mp: 146-148° C.

Mass spectrum: -CI m/z (assignment, relative intensity) 283 (MH⁺, 11%), 265 (MH^{+—H}₂O, 100%), 237 (MH⁺—H₂O—CO, 22%); EI: m/z (assignment, relative intensity) 282 (M⁺, 26%), 209 (M⁺—CH₂COCH₂OH, 100%); High resolution EI Calculated C₁₈H₁₅FO₂ (M⁺): 282.1056, Found: 282.1057 (40%).

### EXAMPLE B32

### (3S,4aR,13bR)-12-fluoro-N,N-dimethyl-2,3,4,4a,9,13b-hexahydrodibenzo[3,4:6,7]-cyclohepta[1,2-b]pyran-3-amine (final compound 32)

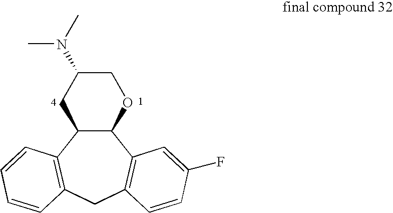

Intermediate compound 54 (0.24 g, 0.76 mmol) was dissolved in i-PrOH/THF (2:1, 15 mL). Add 10% Pd—C (ca. 150 mg) and subject the mixture to hydrogenation (1 atmospheric pressure) for 1 night. Add 35% aq. CH₂O (0.60 mL, 7.6 mmol) and continue hydrogenation for 2 days. Filter through celite and wash with CH₂Cl₂ (5×15 mL). Combine the organic phases and dry with MgSO₄. The solution was filtered and evaporated, and the residue was purified by column chromatography on silica gel using CHCl₃/MeOH (90:10) to yield final compound 32 as an oil (0.22 g, 93%).

Mass spectrum. -CI m/z (assignment, relative intensity) 312 (MH⁺, 100%); EI. m/z (assignment, relative intensity) 311 (M⁺, 7%).

### EXAMPLE B33

### (3R,4aR,13bR)-12-fluoro-N-methyl-2,3,4,4a,9,13b-hexahydrodibenzo[3,4:6,7]cyclo-hepta[1,2-b]pyran-3-amine (final compound 33).

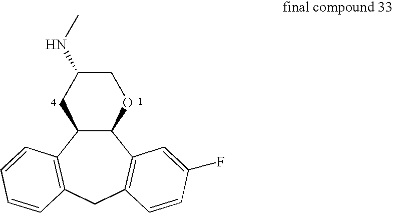

Dissolve final compound 31 (0.18 g, 0.63 mmol) in i-PrOH/THF (2:1, 10 mL). Add 10% Pd—C (ca. 100 mg), Et₃N (0.87 mL, 6.3 mmol) and MeNH2HCl (0.42 g, 6.3 mmol). Subject the mixture to hydrogenation (1 atmospheric pressure) for 1 night. Filter through a pad of celite and wash the solids with CH₂Cl₂ (5×15 mL). The solution was filtered and evaporated and the residue was purified by column chromatography on silica gel using CHCl₃/MeOH (90:10) to yield two diastereoisomers (0.18 g, 95%) with a ratio of 5:1, from which the major (3R)-isomer (final compound 33) can be partly separated.

Mass spectrum: -CI m/z (assignment, relative intensity) 298 (MH⁺, 100 %); EI: m/z (assignment, relative intensity) 297 (M⁺, 5%), 266 (M⁺—CH₃NH₂, 19 %); High resolution EI Calculated C₁₉H₂₀FNO (M⁺): 297.1529, Found: 297.1528 (3.5%).

### EXAMPLE B34

### (4aR*,13bS*)-12-fluoro-4,4a,9,13b-tetrahydrodibenzo[3,4:6,7]cyclohepta[1,2-b]pyran-3(2H)-one (final compound 34)

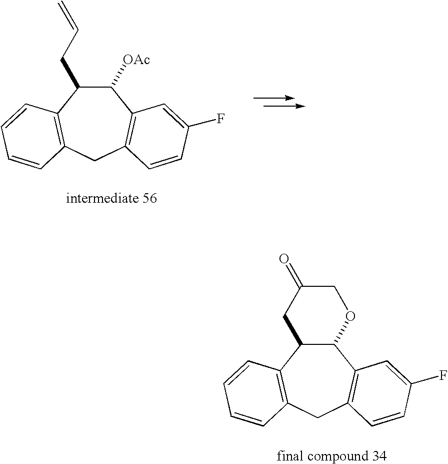

a) Conversion of alkene into diastereoisomeric diols. Dissolve intermediate compound 56 (1.40 g, 4.52 mmol) in acetone (30 mL). Add a small crystal of OsO₄ (catalytic amount) and N-methylmorpholine N-oxide (0.63 g, 5.42 mmol). Stir at room temperature for 1 day. The solvent was removed under reduced pressure, and the residue was purified by column chromatography on silica gel using EtOAc/hexane (80:20) to yield a mixture of two diastereoisomeric diols (oil, 1.45 g, 93%).

b) Selective mono-tosylation of primary alcohol group. Dissolve the above diols (1.45 g, 4.22 mmol) in toluene (50 mL). Add Et₃N (1.76 mL, 12.6 mmol), TsCl (1.05 g, 5.48 mmol) and Bu₂SnO (0.10 g, 0.42 mmol). Stir at room temperature for 1 day. Add sat. aq. NH₄Cl (30 mL), extract with CH₂Cl₂ (3×20 mL) and dry with MgSO₄. The solution was filtered and evaporated and the residue was purified by column chromatography using EtOAc/hexane (40:60) to yield the diastereoisomeric monotosylate derivatives corresponding to selective sulfonylation of the primary OH group (oil, 1.74 g, 83%).

c) Protection of secondary alcohol group. Dissolve the monotosylates (1.74 g, 3.49 mmol) in CH₂Cl₂ (60 mL) and add DHP (1.59 mL, 17.5 mmol), CSA (10 mg, 0.035 mmol). Stir at room temperature for 1 h and remove the solvent under reduced pressure.

d) Deprotection and cyclisation of benzylic alcohol. The residue was dissolved in MeOH (50 mL). Add K₂CO₃ (0.79 g, 6.99 mmol) and stir at room temperature for 1 night. Work up by adding sat. aq. NH₄Cl (30 mL), extract 3 times with CH₂Cl₂ (3×20 niL) and dry with MgSO₄. The solvent was evaporated and the residue containing the benzylic alcohol was dissolved in dry THF (50 mL). Add NaH (0.21 g, 6.99 mmol) and stir at room temperature for 3 days to effect cyclisation. Work it up by adding sat. aq NH₄Cl (30 mL) and extract with CH₂Cl₂ (3×20 mL). Dry with MgSO₄ and evaporate the solvent.

e) Deprotection and oxidation of secondary alcohol group. Dissolve the residue (1.70 g) in 20 mL MeOH/H₂O (9:1) and add Dowex 50WX8-100 (1.00 g). Heat the mixture at 50° C. for 2 hours. Filter through P3 filter, wash the solids with CH₂Cl₂ (5×15 mL) and evaporate. Column purification on silica gel using Et2O/hexane (70:30) yielded an oil (two diastereoisomeric alcohols) (0.87 g, 88%). Dissolve the above oil (0.87 g, 3.06 mmol) in CH₂Cl₂ (40 mL). Add PCC (1.32 g, 6.13 mmol) and stir at room temperature for 1 night. Filter through a pad of celite, wash the solids with CH₂Cl₂ (5×20 mL) and evaporate. Column purification on silica gel using CH₂Cl₂/hexane (80:20) yielded final compound 34 as an oil (0.66 g, 76%).

Mass spectrum: -CI m/z (assignment, relative intensity) 283 (MH⁺, 25%), 265 (M^{H+}—H₂O, 100%); EI: m/z (assignment, relative intensity) 282 (MH⁺, 39%), 209 (M⁺—CH₂COCH₂OH, 100%).

### EXAMPLE B35

### (3S*,4aR*,13bS*)-12-fluoro-N-methyl-2,3,4,4a,9,13b-hexahydrodibenzo[3,4:6,7]-cyclohepta[1,2-b]pyran-3-amine (final compound 35)

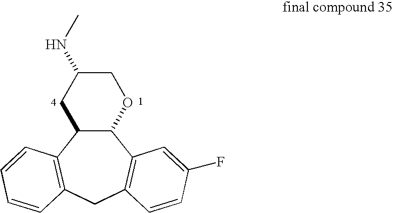

Dissolve final compound 34 (0.23 g, 0.83 mmol) in i-PrOH (15 mL). Add Et₃N (1.15 mL, 8.25 mmol), MeNH₂HCl (0.56 g, 8.25 mmol) and 10% Pd/C (ca. 150 mg). Subject to hydrogenation (1 atmospheric pressure) for 1 night. Filter through a pad of celite and wash the solids with CH₂Cl₂ (5×10 mL). The solution was filtered and evaporated, and the residue was purified by column chromatography on silica gel using CHCl₃/MeOH (90:10) to yield final compound 35 as the nearly exclusive diastereoisomer (0.23 g, 95%).

Mass spectrum: -CI m/z (assignment, relative intensity) 298 (MH⁺, 100%); EI: m/z (assignment, relative intensity) 209 (M⁺—CH₂CH(NHMe)CH₂OH, 100%).

### EXAMPLE B36

a1) Methyl (2R,3aR,12bS)-2-(aminomethyl)-11-fluoro-3,3a,8,12b-tetrahydrodibenzo-[3,4:6,7]-cyclohepta[1,2-b]pyrrole-1 (2H)-carboxylate (intermediate compound 62b), b1) [(2R,3aR,12bS)-1-acetyl-11-fluoro-1,2,3,3a,8,12b-hexahydrodibenzo[3,4:6,7]-cyclohepta[1,2-b]pyrrol-2-yl]methanamine (intermediate compound 62c), and

c1) (2R,3aR,12bS)-2-(aminomethyl)-11-fluoro-3,3a,8,12b-tetrahydrodibenzo[3,4:6,7]-cyclohepta-[1,2-b]pyrrole-1(2H)-carbaldehyde (intermediate compound 62d)

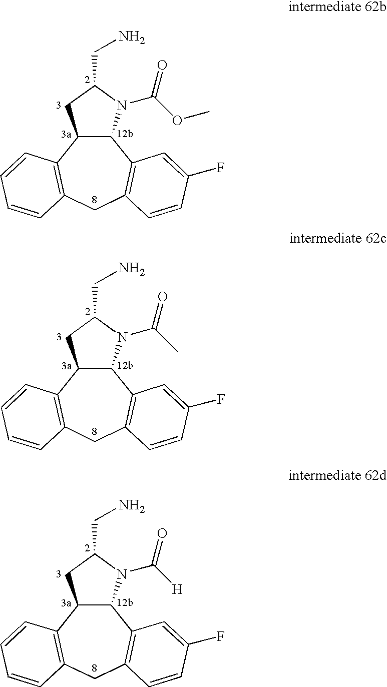

Representative Procedure—Synthesis of Methyl (2R,3aR,12bS)-2-(aminomethyl)-11-fluoro-3,3a8,12b-tetrahydrodibenzo[3.4:6,7]cyclohepta[1,2-b]pyrrole-1(2H)-carboxulate (intermediate compound 62b) : A solution of PPh₃ (996 mg, 3.8 mmol) in dry THF (20 mL) was placed in two-necked 100 mL flask, equipped with septum, argon inlet and magnetic stirrer; cooled down to −15° C. Neat DIAD (768 mg, 3.8 mmol) was added through a septum with intensive stirring. Resulting yellow suspension was stirred at above temperature for 30 minutes, then carbamate intermediate compound 62 (650 mg, 1.9 mmol) in THF (5 mL) was added in one portion. After 5 minutes of stirring, DPPA (606 mg, 2.2 mmol) in THF (3 mL) was added dropwise for 3 minutes, resulting turbid mixture allowed to warm up to room temperature and stirred then for 12 hours. After this time water (0.2 mL) and PPh₃ (996 mg, 3.8 mmol) was added, and solution stirred at 45° C. for 2 hours. After cooling down to room temperature, silica gel (Kieselgel 60, 70-230 mesh, 4 g) was added, THF removed in vacuo, and silica powder submitted to the flash column chromatography (Kieselgel 60, 230-400 mesh, CH₂Cl₂-MeOH, 100/0, gradually to 85/15) to give desired intermediate compound 62b (401 mg, 1.18 mmol, 62%) as colorless oil, darkening on standing.

### Intermediate Compound 62b

HRMS Calcd. for C₂₀H₂₁FN₂O₂: 340.1587; Found: 340.1588.

### Intermediate Compound 62c: Two Rotamers Present (ca. 2:1 Ratio)

CI-MS (CH₄) 325 (MH⁺, 100%); 305 (MH⁺—HF, 10%). HRMS Calcd. for C₂₀H₂₁FN₂O: 324.1638; Found. 324.1644.

### Intermediate Compound 62d: Two Rotamers Present (ca. 5:2 Ratio)

HRMS Calcd. for C₁₉H₁₉FN₂O: 310.1481; Found. 310.1480.

a2) Methyl (2R,3aR, 12bS)-2-[(dimethylamino)methyl]-11-fluoro-3,3a,8,12b-tetrahydro dibenzo[3,4:6,7]cyclohepta[1,2-b]pyrrole-1(2H)-carboxylate (final compound 36a),

b2) [(2R,3aR, 1 2bS)-1-acetyl-11-fluoro-1,2,3,3a,8,12b-hexahydrodibenzo[3,4:6,7]cyclohepta[1,2-b]pyrrol-2-yl]-N,N-dimethylmethanamine (final compound 36b) and

c2) (2R,3aR, 1 2bS)-2-[(dimethylamino)methyl]-11-fluoro-3,3a,8,12b-tetrahydrodibenzo [3,4:6,71cyclohepta[1,2-b]pyrrole-1(2H)-carbaldehyde (final compound 36c)

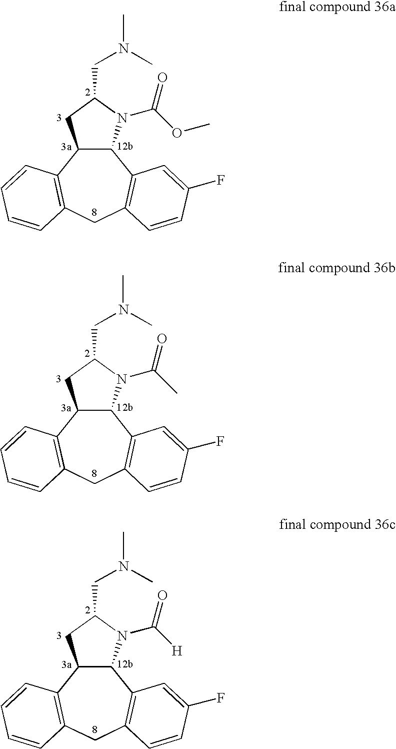

Representative procedure—Synthesis of Methyl (2R,3aR,12bS)-2-[(dimethylamino) methyl]-11-fluoro-3,3a,8,12b-tetrahydrodibenzo[3 4:6,7]cyclohepta[1,2-b]pyrrole-1(2H)-carboxylate (final compound 36a): Intermediate compound 62b (401 mg, 1.18 mmol) was dissolved in MeOH (30 mL), AcOH (1 mL) and 35% aqueous formaldehyde (1 g, 11.7 mmol) added, followed by NaCNBH₄ (628 mg, 10 mmol). The resulting mixture was stirred at room temperature for 4 hours, quenched with concentrated HCl (5 mL), treated with solid NaHCO₃ (8.4 g, 100 mmol), 1N NaOH (15 mL). The precipitated product was filtered off, washed with water (5×25 mL), dissolved in EtOAc, washed with brine (30 mL), dried (K₂CO₃), evaporated in vacuo and purified by column chromatography (Kieselgel 60 , 230-400 mesh, CH₂Cl2-MeOH 95/5 to 90/10 to 85/15) to give final compound 36a (313 mg, 0.85 mmol, 72%) as yellowish oil.

### Final Compound 36a:

HRMS Calcd. for C₂₂H₂₅FN₂O₂: 368.1900; Found: 368.1895.

### Final Compound 36b: Two Rotamers, ca. 3:2 Ratio.

HRMS Calcd. for C₂₂H₂₅FN₂O: 352.1951; Found: 352.1955.

### Final Compound 36c: Two Rotamers, ca. 5:3 Ratio.

HRMS Calcd. for C₂₁H₂₃FN₂O 338.1794; Found: 338.1790.

### EXAMPLE 37

### [(2R,3aR,12bS)-11-fluoro-1,2,3,3a,8,12b-hexahydrodibenzo[3,4:6,7]cyclohepta[1,2-b]-pyrrol-2-yl]-N,N-dimethylmethanamine (final compound 37)

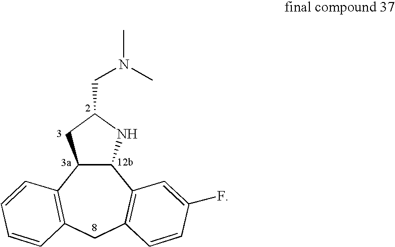

A mixture of final compound 36a (100 mg, 0.27 mmol), i-PrOH (10 mL), KOH (560 mg, 10 mmol) and water (0.1 mL) was refluxed under N₂ atmosphere for 12 hours (oil bath temperature 135° C.), then cooled to room temperature. After dilution with water (50 mL), extraction with EtOAc (3×40 mL), the combined organics were washed with water (3×40 mL), brine (40 mL), dried over K₂CO₃ and evaporated to give pure final compound 37 (84 mg, 100%) as yellowish semisolid, which was converted to the hydrochloride salt (final compound 37a).

HRMS Calcd. for C₂₀H₂₃FN₂: 310.1845; Found: 310.1851.

### EXAMPLE B38

### (2R,3aR,12bS)-11-fluoro-2-[(methylamino)methyll]-3,3a,8,12b-tetrahydrodibenzo-[3,4:6,7]cyclohepta[1,2-b]pyrrole-1 (2H)-carbaldehyde (final compound 38)

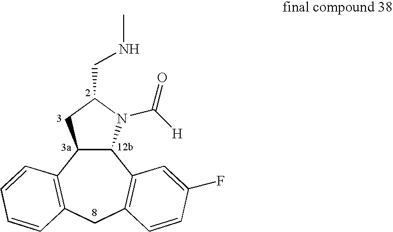

A mixture of intermediate compound 63 (50 mg, 0.162 mmol), methylamine hydrochloride (218 mg, 3.24 mmol), Et₃N (405 mg, 4.0 mmol), 10% Pd—C (30 mg) and MeOH (12 mL) was hydrogenated for 2 hours at atmospheric pressure. The reaction mixture was filtered through Kieselguhr, which was subsequently washed with EtOAc (2×10 mL). The combined solutions were evaporated in vacuo and residue was purified by column chromatography (Kieselgel 60, 70-230 mesh, CH₂Cl₂/MeOH 100/0 to 85/15) to give final compound 38 (21 mg, 0.065 mmol, 40%) as brown oil;Four rotamers present (10:6:4:1 ratio).

CI-MS (CH₄): 325 (100%, M+H⁺), 305 (12%, —HF). HRMS Calcd. for C₂₀H₂₁FN₂O: 324.1638; Found: 324.1650.

### EXAMPLE B39

### 2-((2R,3aR,12bS)-2-[(dimethylamino)methyl]-11-fluoro-3,3a,8,12b-tetrahydrodibenzo-[3,4:6,7]cyclohepta[1,2-b]pyrrol-1(2H)-yl)ethanol (final compound 39)

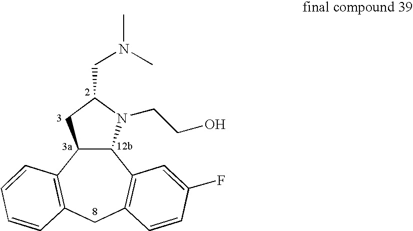

Hydroxyacetaldehyde dimer (2,5-dihydroxy-1,4-dioxane) (240 mg, 2.0 mmol) was dissolved in MeOH (25 mL) and stirred at 40° C. for 30 minutes, then final compound 37 (124 mg, 0.40 mmol) was added and stirring at 40° C. continued for another 30 minutes. After cooling down to room temperature, AcOH (120 mg, 2.0 mmol) was added, followed by sodium cyanoborohydride (188 mg, 3.0 mmol) and the resulting mixture was stirred for 2 hours. After this time it was quenched with concentrated HCl (2 mL), treated with solid NaHCO₃ (2.94 g, 35 mmol), 1N NaOH (3 mL). About 20 mL of MeOH was removed in vacuo, the residue diluted with water (30 mL), and extracted with EtOAc (3×30 mL). The combined organics were washed with water (5×25 mL), brine (30 mL), dried (K₂CO₃), evaporated in vacuo and purified by column chromatography (Kieselgel 60, 230-400 mesh, CH₂Cl₂-MeOH 95/5 to 90/10 to 85/15) to give the final compound 39 (80 mg, 0.244 mmol, 61%) as colorless oil.

HRMS Calcd. for C₂₂H₂₇FN₂O: 354.2107; Found: 354.2107.

### EXAMPLE B40

### [(2R,3aR,12bS)-11-Fluoro-1-(2-methoxyethyl)-1,2,3,3a,8,12b-hexahydrodibenzo-[3,4:6,7]-cyclohepta[1,2-b]pyrrol-2-yl]-N,N-dimethylmethanamine (final compound 40)

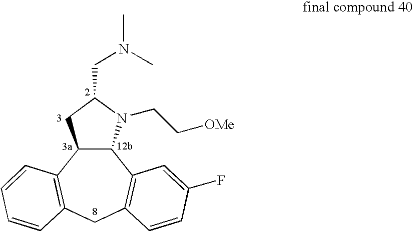

Final compound 39 (50 mg, 0.153 mmol) was dissolved in dry THF (10 mL), then 60% NaH dispersion (8 mg, 0.2 mmol) was added, followed by dimethyl sulfate (25 mg, 0.2 mmol). The resulting mixture was stirred under argon atmosphere at 60° C. for 5 hours, then cooled, quenched with concentrated NH₄OH (2 mL), diluted with water (40 mL). After extraction of product with EtOAc (3×25 mL) the combined organics were washed with water (3×25 mL), brine (25 mL), dried over K₂CO₃, evaporated in vacuo, and the residue purified by column chromatography (Kieselgel 60, 230-400 mesh, CH₂Cl₂-MeOH 95/5 to 90/10 to 85/15) to give final compound 40 (39 mg, 0.107 mmol, 70%) as yellowish oil.

HRMS Calcd. for C₂₃H₂₉FN₂O: 368.2264; Found: 368.2270.

### EXAMPLE B41

### [(2R,3aR,12bS)-1-cyano-11-fluoro-1,2,3,3a,8,12b-hexahydrodibenzo[3,4:6,7]cyclohepta[1,2-b]pyrrol-2-yl]-N,N-dimethylmethanamine (final compound 41)

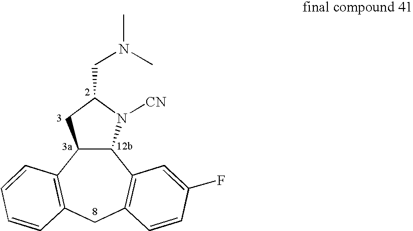

Poly(4-vinylpyridine) crosslinked with 2% divinylbenzene (0.5 g) was swollen for 1 hour with CH₂Cl₂ (10 mL), then final compound 37 (57 mg, 0.184 mmol) in CH₂Cl₂ (2 mL) was added in one portion, followed by cyanogen bromide (39 mg, 0.367 mmol), then suspension stirred at room temperature for 30 minutes. The resin was filtered off, filtrate treated with sat. aq. K₂CO₃ (10 mL), organic phase was separated, evaporated in vacuo, and the residue purified by column chromatography (Kieselgel 60, 230-400 mesh, CH₂Cl₂-MeOH 95/5 to 90/10→87/13) to give final compound 41 (24 mg, 0.077 mmol, 42%) as brownish oil.

CI-MS (CH₄): 308 (100%, M+H⁺), 288 (8%, —HF). HRMS Calcd. for C₁₉H₁₈FN₃: 307.1485; Found: 307.1499.

### EXAMPLE B42

### (2R,3aR,12bS)-11-fluoro-2-(4-morpholinylmethyl)-1,2,3,3a,8,12b-hexahydrodibenzo-[3,4:6,7]cyclohepta[1,2-b]pyrrole (final compound 42)

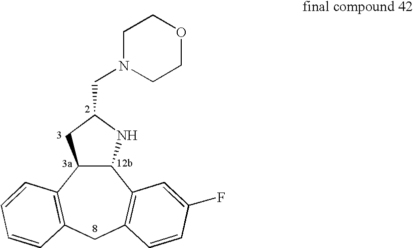

To a solution of intermediate compound 64 (63 mg, 0.237 mmol) in acetonitrile (1 mL) was added Nal (107 mg, 0.711 mmol) and trimethylsilyl chloride (90 μL, 0.711 mmol) at room temperature. After the solution was stirred for 2 hours, morpholine (44 mg, 0.5 mmol) in acetonitrile (0.5 mL) was added dropwise to the mixture. The solution was heated to the boiling point of the solvent for 2 hours. The dark brown reaction mixture was quenched with aqueous 1.2 N HCl solution and then was treated with sat. Na—HCO₃. The organic layer was separated and the aqueous layer was extracted with CH₂Cl₂ (3×10 mL). The combined organic extracts were washed with 20 mL of brine, dried over anhydrous MgSO₄, filtered and concentrated in vacuo. The residue was purified by column chromatography on basic alumina (Brockmann III, EtOAc-MeOH, 100/0 to 98/2 to 95/5) gave final compound 42 (38 mg, 0.11 mmol, 45%) as brownish oil.

CI-MS (CH₄): 353 (100%, M+H⁺); 333 (—HF, 7%). HRMS Calcd. for C₂₂H₂₅FN₂O: 352.1951; Found: 352.1966.

### EXAMPLE B43

### 2-(4-{[(2R,3aR,12bS)-11-fluoro-1,2,3,3a,8,12b-hexahydrodibenzo[3,4:6,7]cyclohepta-[1,2-b]pyrrol-2-yl]methyl}-1-piperazinyl)ethanol (final compound 43a) and 2-[{[(2R,3aR,12bS)-11-fluoro-1,2,3,3a,8,12b-exahydrodibenzo[3,4:6,7]cyclohepta[1,2-b]pyrrol-2-yl]methyl}(methyl)amino]-ethanol (final compound 43b)

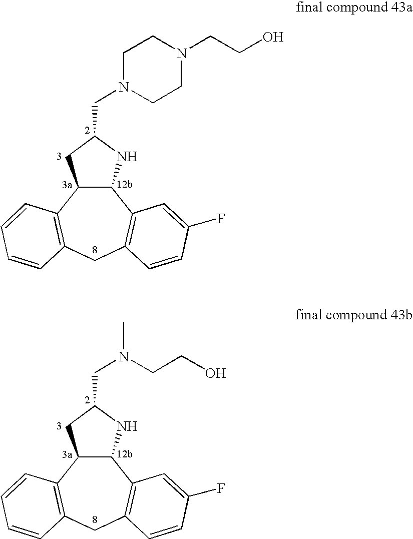

A mixture of appropriate intermediate compound 66a or 66b (ca. 0.4 mmol), thiophenol (110 mg, 1.0 mmol), anhydrous K₂CO₃ (138 mg, 1 mmol) and DMF (20 mL) was stirred at 80° C. for 4 hours, cooled to ambient temperature, diluted with water, product extracted with EtOAc (3×50 mL), combined organics washed with water (4×50 mL), brine (35 mL), dried (K₂CO₃), evaporated and purified by solid phase extraction on basic alumina (Brockmann II, heptane-ethyl acetate 50/50, then EtOAc-MeOH 100/0 to 96/4 to 90/10) to give final compound 43a (111 mg, 0.28 mmol, 52% from intermediate compound 65) or final compound 43b (80 mg, 0.24 mmol, 44% from intermediate compound 65), both as brownish oils.

### Final Compound 43a (TK-895):

HRMS: Calcd. for C₂₄H₃₀FN₃O: 395.2373; Found: 395.2374.

### Final Compound 43b (TK-1013):

HRMS Calcd. for C₂₁H₂₅FN₂O: 340.1951; 340.1943.

### EXAMPLE B44

### [(2R,4aR,13bS)-12-fluoro-2,3,4,4a,9,13b-hexahydro-1H-dibenzo[3,4:6,7]cyclohepta-[1,2-b]pyridin-2-yl]-N,N-dimethvlmethanamine (final compound 44)

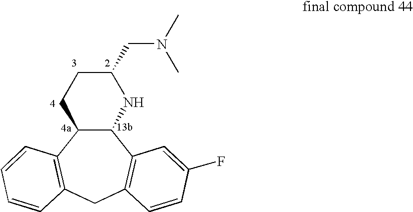

Intermediate compound 67k (153 mg, 0.338 mmol), 40% aqueous methylamine (15 mL), and THF (35 mL) were heated in stainless-steel bomb at 135° C. for 15 hours. After cooling, the bomb was opened, THF and methylamine evaporated in vacuo, residue extracted with CH₂Cl₂ (4×20 mL). The combined organics were washed with water (3×20 mL), dried (K₂CO₃), evaporated and purified by column chromatography (Kieselgel 60, 230-400 mesh, CH₂Cl₂-MeOH 98/2 to 85/15) to afford final compound 44 (32 mg, 0.098 mmol, 29%) as a brown oil, which was converted to the oxalate salt (final compound 44a).

HRMS Calcd. for C₂₁H₂₅FN₂: 324.2002; Found: 324.1995.

### EXAMPLE B45

### Methyl (2R,4aR,13bS)-2-[(dimethylamino)methyl]-12-fluoro-2,3,4,4a,9,13b-hexahydro-1H-dibenzo[3,4:6,7]cyclohepta[1,2-b]pyridine-1-carboxylate (final compound 45)

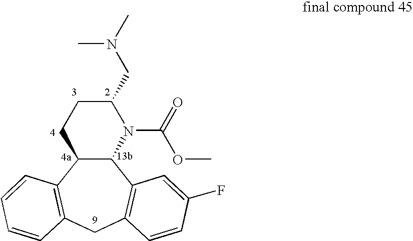

Conversion of final compound 44 (48 mg, 0.15 mmol) with methyl chloroformate was carried out in the same way as described for the preparation of intermediate compound 62. Column chromatography (Kieselgel 60, 70-230 mesh, MeOH—CH₂Cl₂ 3/97 to 15/85) afforded final compound 45 (45 mg, 0.118 mmol, 79%) as colorless oil. HRMS Calcd. for C₂₃H₂₇FN₂O₂: 382.2057; Found: 382.2064.

### EXAMPLE B46

### [(2R,4aR,13bS)-12-fluoro-2,3,4,4a,9,13b-hexahydrodibenzo[3,4:6,7]cyclohepta-[1,2-b]pyran-2-yl]-N-methylmethanamine (final compound 46)

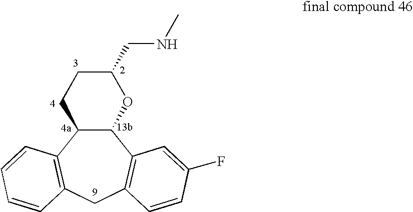

Intermediate compound 68e (282 mg, 0.62 mmol), 40% aqueous methylamine (25 mL), and THF (35 mL) were heated in a steel bomb at 135° C. for 15 hours. After cooling, bomb was opened, THF and methylamine evaporated in vacuo. The residue was extracted with CH₂Cl₂ (4×30 mL) and the combined organics were washed with water (3×20 mL), dried (K₂CO₃) and evaporated. Crystallization from CH₂Cl₂/hexane gave final compound 46 (70 mg, 0.225 mmol, 36%) as beige powder. HRMS Calcd. for C₂₀H₂₂FNO: 311.1685; Found: 311.1700.

### EXAMPLE B47

### [(3aR,12bS)-11-fluoro-3,3a,8,12b-tetrahydro-2H-dibenzo[3,4:6,7]cyclohepta[1,2-b]-furan-3-yl]-N,N-dimethylmethanamine (final compound 47)

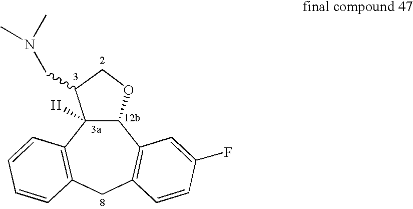

Intermediate compound 69i (122 mg, 0.39 mmol) was dissolved in MeOH (10 mL), 10% palladium on carbon (40 mg) was added and the mixture submitted to the hydrogenation under atmospheric pressure for 1.5 hour, then 35% aqueous formaldehyde (1 g) and AcOH (120 mg, 2 mmol) were added, and hydrogenation continued for 2 hours. After filtration through short pad of Celite, and addition of EtOAc (45 mL), the reaction mixture was washed with sat. aq. NaHCO₃ (25 mL), water (2×50 mL), brine (30 mL), dried over K₂CO₃ and evaporated in vacuo. The residue was purified by column chromatography (Kieselgel 60, 70-230 mesh, ethyl acetate-MeOH, 100/0 to 95/5 to 92/8 to 87/13) to afford final compound 47 (77 mg, 0.248 mmol, 63%) as yellow oil. Product is a mixture of 2 epimers (12.8:1 ratio).

CI-MS (CH₄) 312 (MH^{+,} 100%); 292 (MH⁺—HF, 9%). HRMS Calcd. for C₂₀H₂₂FNO: 311.1685; Found: 311.1680.

### EXAMPLE B48

### [(3aR,12bR)-11-fluoro-3,3a,8,12b-tetrahydro-2H-dibenzo[3,4:6,7]cyclohepta[1,2-b]-furan-3-yl]-N,N-dimethylmethanamine (final compound 48)

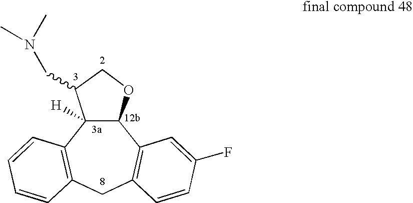

Reaction of intermediate compound 70d (100 mg, 0.304 mmol) was carried out was carried out in the same way as described for final compound 47. Purification by solid phase extraction (Alltech C₁₈ 2 g cartridge, wter-MeOH, 100/0 to 50/50 to 0/100) furnished final compound 48 (57 mg, 0.18 mmol, 59%). Product is a mixture of 2 epimers (2:1 ratio).

CI-MS (CH₄) 312 (MH⁺, 100%); 292 (MH⁺—HF, 12%). HRMS Calcd. for C₂₀H₂₂FNO: 311.1685, Found: 311.1692.

### EXAMPLE B49

### [(3aR,12bS)-11-fluoro-1,2,3,3a,8,12b-hexahydrodibenzo[3,4:6,7]cyclohepta[1,2-b]-pyrrol-3-yl]-N,N-dimethylmethanamine (final compound 49)

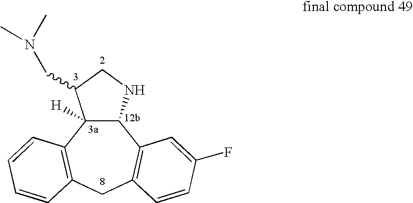

A mixture of intermediate compound 71f (194 mg, 0.53 mmol) and 10% palladium on carbon (50 mg) in MeOH (35 mL) was hydrogenated at atmospheric pressure for 40 minutes, then 35% aqueous formaldehyde (1 mL) was added and hydrogenation continued for another 40 minutes. After filtration through short pad of Celite, reaction mixture was evaporated in vacuo. The residue was dissolved i-PrOH (20 mL), KOH (560 mg, 10 mmol) and water (0.1 mL) were added and resulting solution was refluxed under nitrogen atmosphere for 12 hours (oil bath temperature 135° C.), then cooled to room temperature. After dilution with water (50 mL), extraction with EtOAc (3×40 mL), the combined organics were washed with water (3×40 mL), brine (40 mL), dried over K₂CO₃ and evaporated in vacuo. The residue was purified by column chromatography (basic alumina, Brockmann activity I, EtOAc-MeOH, 100/0 to 85/15) to give pure final compound 49 (102 mg, 0.33 mmol, 62%) as brown oil. Product is a mixture of 2 epimers (1:1 ratio)

HRMS Calcd. for C₂₀H₂₃FN₂: 310.1845; Found: 310.1833.

Tables 1-3 list compounds of Formula (I), which were prepared according to one of the above examples.

TABLE 1

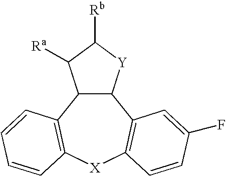

| Co.No. | Ex.No. | X | Y | 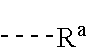 | 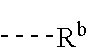 | Stereochemical/salt data |
|---|---|---|---|---|---|---|
| 17 | B17 | —O— | 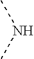 | 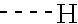 | 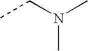 | 2R,3aR,12bS |
| 18 | B18 | —O— | 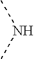 | 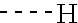 | 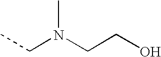 | 2R,3aR,12bS |
| 19 | B19 | —O— | 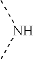 | 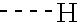 | 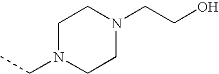 | 2R,3aR,12bS |
| 20 | B20 | —O— | 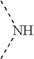 | 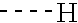 | 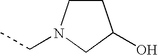 | 2R,3aR,12bS |
| 2 | B2 | —CH₂— | 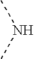 | 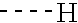 | 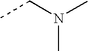 | 2S,3aR,12bS |
| 37 | B37 | —CH₂— | 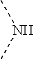 | 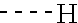 | 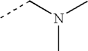 | 2R,3aR,12bS |
| 37a | B37 | —CH₂— | 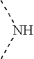 | 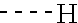 | 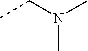 | •HCl2R,3aR,12bS |
| 11 | B11 | —CH₂— | 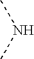 | 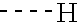 | 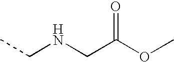 | l2R,3aR,12bS |
| 43b | B43 | —CH₂— | 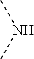 | 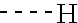 | 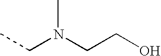 | 2R,3aR,12bS |
| 42 | B42 | —CH₂— | 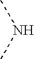 | 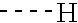 | 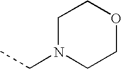 | 2R,3aR,12bS |
| 43a | B43 | —CH₂— | 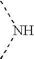 | 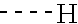 | 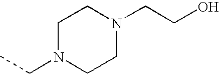 | 2R,3aR,12bS |
| 5 | B5 | —CH₂— | 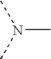 | 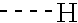 | 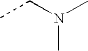 | 2R,3aR,12bS |
| 39 | B39 | —CH₂— | 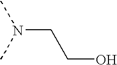 | 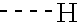 | 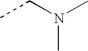 | 2R,3aR,12bS |
| 40 | B40 | —CH₂— | 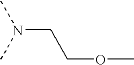 | 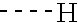 | 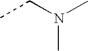 | 2R,3aR,12bS |
| 36c | B36c | —CH₂— | 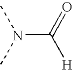 | 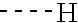 | 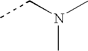 | 2R,3aR,12bS |
| 38 | B38 | —CH₂— | 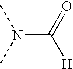 | 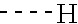 | 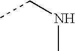 | 2R,3aR,12bS |
| 41 | B41 | —CH₂— | 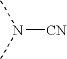 | 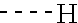 | 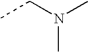 | 2R,3aR,12bS |
| 36b | B36 | —CH₂— | 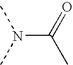 | 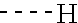 | 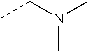 | 2R,3aR,12bS |
| 10 | B10 | —CH₂— | 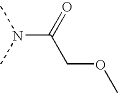 | 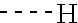 | 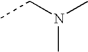 | 2R,3aR,12bS |
| 36a | B36 | —CH₂— | 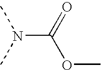 | 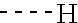 | 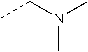 | 2R,3aR,12bS |
| 21 | B21 | —CH₂— | 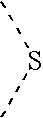 | 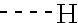 | 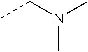 | 2S,3aR,12bS |
| 23 | B23 | —CH₂— | 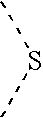 | 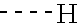 | 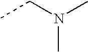 | 2R,3aR,12bS |
| 25 | B25 | —CH₂— | 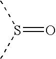 | 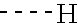 | 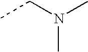 | 2S,3aR,12bS |
| 26 | B26 | —CH₂— | 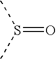 | 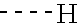 | 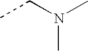 | 2S,3aR,12bS |
| 27 | B27 | —CH₂— | 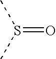 | 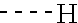 | 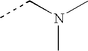 | 2R,3aR,12bS |
| 28 | B28 | —CH₂— | 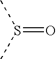 | 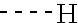 | 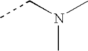 | 2R,3aR,12bS |
| 22 | B22 | —CH₂— | 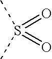 | 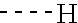 | 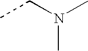 | 2S,3aR,12bS |
| 24 | B24 | —CH₂— | 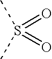 | 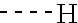 | 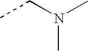 | 2R,3aR,12bS |
| 49 | B49 | —CH₂— | 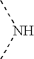 | 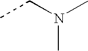 | 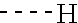 | 3aR,12bS |

TABLE 2

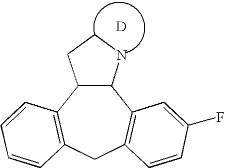

| Co. No. | Ex. No. | D | Stereochemical/salt data |
|---|---|---|---|
| 1 | B1 | 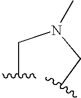 | 4aS,13bR,14aS |
| 6 | B6 | 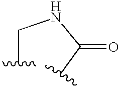 | 4aS,13bR,14R |
| 15 | B15 | 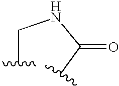 | 4aS,13bR,14aS |
| 7 | B7 | 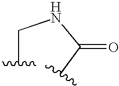 | 4aS,13bR,14R |
| 16 | B16 | 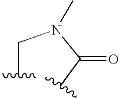 | 4aS,13bR,14S |
| 8 | B8 | 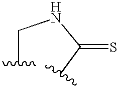 | 4aS,13bR,14R |
| 3 | B3 | 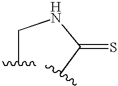 | 4aS,13bR,14S |
| 9 | B9 | 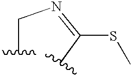 | 4aS,13bR,14R |
| 4 | B4 | 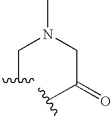 | 5aS,14bR,15aS |
| 13 | B13 | 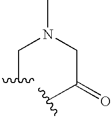 | 5aS,14bR,15aR |
| 12 | B12 | 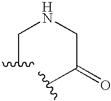 | 5aS,14bR,15aR |
| 14 | B14 | 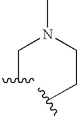 | 5aS,14bR,15aR |
| 14a | B14 | 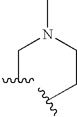 | Oxalate (1:1)5aS,14bR,15aR |

TABLE 3

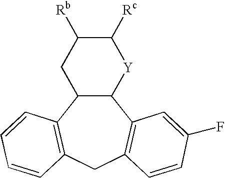

| Co. | Ex. | | | | |
|---|---|---|---|---|---|
| No. | No. | 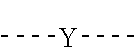 | 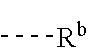 | 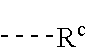 | Stereochemical/salt data |
| 29 | B29 | 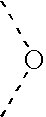 | 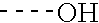 | 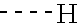 | 3R,4aR,13bR |
| 31 | B31 | 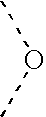 | ═O | 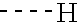 | 4aR,13bR |
| 34 | B34 | 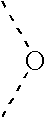 | ═O | 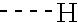 | 4aR*,13bS* |
| 30 | B30 | 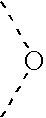 | 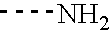 | 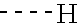 | 3S,4aR,13bR |
| 33 | B33 | 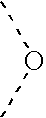 | 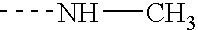 | 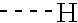 | 3R,4aR,13bR |
| 35 | B35 | 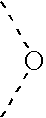 | 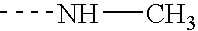 | 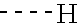 | 3S*,4aR*,13bS* |
| 32 | B32 | 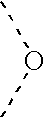 | 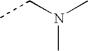 | 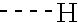 | 3S,4aR,13bR |
| 46 | B46 | 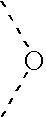 | 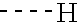 | 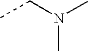 | 2R,4aR,13bS |
| 44 | B44 | 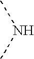 | 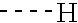 | 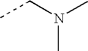 | 2R,4aR,13bS |
| 44a | B44 | 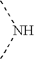 | 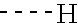 | 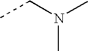 | oxalate (1:2)2R,4aR,13bS |
| 45 | B45 | 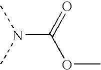 | 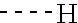 | 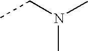 | 2R,4aR,13bS |

### C. Pharmacological Examlpes

### EXAMPLE C.1

### In Vitro Binding Affinity for 5-HT_{2A} and 5-HT_{2C} Receptors

The interaction of the compounds of Formula (I) with 5-HT_{2A} and 5-HT_{2C} receptors was assessed in in vitro radioligand binding experiments. In general, a low concentration of a radioligand with a high binding affinity for the receptor is incubated with a sample of a tissue preparation enriched in a particular receptor (1 to 5 mg tissue) in a buffered medium (0.2 to 5 ml). During the incubation, the radioligands bind to the receptor. When equilibrium of binding is reached, the receptor bound radioactivity is separated from the non-bound radioactivity, and the receptor bound activity is counted. The interaction of the test compounds with the receptors is assessed in competition binding experiments. Various concentrations of the test compound are added to the incubation mixture containing the tissue preparation and the radioligand. Binding of the radioligand will be inhibited by the test compound in proportion to its binding affinity and its concentration. The affinities of the compounds for the 5-HT₂ receptors were measured by means of radioligand binding studies conducted with: (a) human cloned 5-HT_{2A} receptor, expressed in L929 cells using [¹²⁵I]R91150 as radioligand and (b) human cloned 5-HT_{2C} receptor, expressed in CHO cells using [³H]mesulergine as radioligand.

### EXAMPLE C.2

### In Vitro Determination of NET Reuptake Inhibition

Cortex from rat brain was collected and homogenised using an Ultra-Turrax T25 and a Dual homogeniser in ice-cold homogenising buffer containing Tris, NaCl and KCl (50 mM, 120 mM and 5 mM, respectively, pH 7.4) prior to dilution to an appropriate protein concentration optimised for specific and non-specific binding. Binding was performed with radioligand [³H]Nixosetine (NEN, NET-1084, specific activity ˜70 Ci/mmol) diluted in ice cold assay buffer containing Tris, NaCl and KCl (50 mM, 300 mM and 5 mM, respectively, pH 7.4). at a concentration of 20 nmol/L. Prepared radioligand (50 μl) was then incubated (60 min, 25° C.) with membrane preparations prediluted to an appropriate protein concentration (400 μl), and with 50 μl of either the 10% DMSO control, Mazindol (10-6 mol/L final concentration), or compound of interest. Membrane-bound activity was detected by filtration through a Packard Filtermate harvester onto GF/B Unifilterplates, washed with ice-cold Tris-HCl buffer, containing NaCl and KCl (50 mM, 120 mM and 4 mM; pH 7.4; 6×0.5 ml). Filters were allowed to dry for 24 h before adding scintillation fluid. Scintillation fluid was allowed to saturate filters for 24 h before counting in a Topcount scintillation counter. Percentage specific bound and competition binding curves were calculated using S-Plus software (Insightful).

### EXAMPLE C.3

### In Vitro Binding Affinity for Human D2_{L} Receptor

Frozen membranes of human Dopamine D2_{L} receptor-transfected CHO cells were thawed, briefly homogenised using an Ultra-Turrax T25 homogeniser and diluted in Tris-HCl assay buffer containing NaCl, CaCl₂, MgCl₂, KCl (50, 120, 2, 1, and 5 mM respectively, adjusted to pH 7.7 with HCl) to an appropriate protein concentration optimised for specific and non-specific binding. Radioligand [³H]Spiperone (NEN, specific activity ˜70 Ci/mmol) was diluted in assay buffer at a concentration of 2 nmol/L. Prepared radioligand (50 μl), along with 50 μl of either the 10% DMSO control, Butaclamol (10-6 mol/l final concentration), or compound of interest, was then incubated (30 min, 37° C.) with 400 μl of the prepared membrane solution. Membrane-bound activity was filtered through a Packard Filtermate harvester onto GF/B Unifilterplates and washed with ice-cold Tris-HCl buffer (50 mM; pH 7.7; 6×0.5 ml). Filters were allowed to dry before adding scintillation fluid and counting in a Topcount scintillation counter. Percentage specific bound and competition binding curves were calculated using S-Plus software (Insightful).

TABLE 4
Pharmacological data.

| Co. No. | h-5HT_{2A} | h-5HT_{2C} | h-D2L | NET Reuptake Inhibition |
|---|---|---|---|---|
| 17 | 6.24 | 6.30 | 5.63 | 8.13 |
| 37a | 7.35 | 7.30 | 6.45 | 8.10 |
| 47 | 5.42 | 5.80 | n.d. | 7.96 |
| 43b | 7.17 | 7.05 | 6.36 | 7.80 |
| 32 | 6.17 | 6.88 | <6 | 7.71 |
| 23 | 6.18 | 6.64 | 5.30 | 7.55 |
| 1 | 6.94 | 6.82 | 5.65 | 6.94 |
| 39 | 7.06 | 7.33 | <6 | 6.90 |
| 28 | 5.11 | 5.75 | n.d. | 6.84 |
| 48 | 5.21 | 5.52 | n.d. | 6.65 |
| 36c | 6.26 | 7.11 | 5.31 | 6.65 |
| 5 | 7.56 | 8.27 | 6.88 | 6.54 |
| 30 | 6.57 | 6.84 | <6 | 6.52 |
| 46 | 7.86 | 8.23 | 5.20 | 6.40 |
| 20 | n.d. | 6.96 | 6.45 | 6.38 |
| 40 | 6.43 | 6.58 | <6 | 6.32 |
| 38 | 6.20 | 6.73 | 5.15 | 6.31 |
| 36a | <6 | <6 | <6 | 6.16 |
| 45 | n.d. | 5.65 | <5 | 6.10 |
| 7 | <6 | <6 | <6 | 6.05 |
| 8 | 7.07 | 6.60 | <5 | 5.66 |
| 15 | 5.08 | 5.63 | <5 | 5.62 |
| 25 | <5 | 5.65 | n.d. | 5.54 |
| 14a | 8.90 | 9.05 | 8.81 | 5.50 |
| 12 | n.d. | 7.23 | 6.08 | 5.46 |
| 36b | <6 | <6 | <6 | 5.41 |
| 9 | <6 | <6 | <6 | 5.40 |
| 22 | 5.07 | 5.87 | n.d. | 5.32 |
| 10 | 6.16 | 6.37 | <6 | 5.32 |
| 42 | 6.20 | 6.26 | n.d. | 5.26 |
| 3 | <6 | 6.62 | <6 | 5.24 |
| 13 | 7.06 | 6.92 | 6.37 | <5 |
| 35 | <6 | 5.58 | <6 | <6 |
| 43a | 6.37 | 6.39 | n.d. | <5 |
| 26 | <5 | <5 | <5 | <5 |
| 19 | n.d. | 5.37 | 6.95 | <5 |
| 16 | <5 | <5 | <5 | <5 |
| 4 | <6 | <6 | <6 | <5 |

n.d. = not determined

### D. Composition Examples

“Active ingredient” (A.I.) as used throughout these examples relates to a compound of Formula (I), a pharmaceutically acceptable acid addition salt, a stereochemically isomeric form thereof or a N-oxide form thereof.

### EXAMPLE D.1

### Oral Solution

Methyl 4-hydroxybenzoate (9 g) and propyl 4-hydroxybenzoate (1 g) were dissolved in boiling purified water (4 l). In 3 l of this solution were dissolved first 2,3-dihydroxybutanedioic acid (10 g) and thereafter A.I (20 g). The latter solution was combined with the remaining part of the former solution and 1,2,3-propanetriol (12 l) and sorbitol 70% solution (3 l) were added thereto. Sodium saccharin (40 g) were dissolved in water (500 ml) and raspberry (2 ml) and gooseberry essence (2 ml) were added. The latter solution was combined with the former, water was added q.s. to a volume of 20 l providing an oral solution comprising 5 mg of the active ingredient per teaspoonful (5 ml). The resulting solution was filled in suitable containers.

### EXAMPLE D.2

### Film-Coated Tablets

### Preparation of Tablet Core

A mixture of A.I. (100 g), lactose (570 g) and starch (200 g) was mixed well and thereafter humidified with a solution of sodium dodecyl sulfate (5 g) and polyvinylpyrrolidone (10 g) in water (200 ml). The wet powder mixture was sieved, dried and sieved again. Then there was added microcrystalline cellulose (100 g) and hydrogenated vegetable oil (15 g). The whole was mixed well and compressed into tablets, giving 10.000 tablets, each containing 10 mg of the active ingredient.

### Coating

To a solution of methyl cellulose (10 g) in denaturated ethanol (75 ml) there was added a solution of ethyl cellulose (5 g) in dichloromethane (150 ml). Then there were added dichloromethane (75 ml) and 1,2,3-propanetriol (2.5 ml). Polyethylene glycol (10 g) was molten and dissolved in dichloromethane (75 ml). The latter solution was added to the former and then there were added magnesium octadecanoate (2.5 g), polyvinylpyrrolidone (5 g) and concentrated colour suspension (30 ml) and the whole was homogenated. The tablet cores were coated with the thus obtained mixture in a coating apparatus.

### EXAMPLE D.3

### Injectable Solution

Methyl 4-hydroxybenzoate (1.8 g) and propyl 4-hydroxybenzoate (0.2 g) were dissolved in boiling water (500 ml) for injection. After cooling to about 50° C. there were added while stirring lactic acid (4 g), propylene glycol (0.05 g) and A.I. (4 g). The solution was cooled to room temperature and supplemented with water for injection q.s. ad 1000 ml, giving a solution comprising 4 mg/ml of A.I. The solution was sterilized by filtration and filled in sterile containers.

1. A compound according to Formula (I) 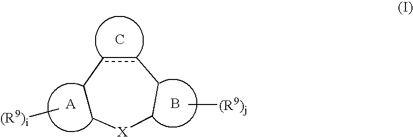
an N-oxide form, a pharmaceutically acceptable addition salt or a stereochemically isomeric form thereof, wherein:
the dotted line represents an optional bond;
i and j are integers, independently from each other, equal to zero, 1, 2, 3 or 4
A and B are, each independently from each other, aryl or an heteroaryl radical selected from the group of furyl; thienyl; pyrrolyl; oxazolyl; thiazolyl; imidazolyl; isoxazolyl; isothiazolyl; oxadiazolyl; triazolyl; pyridinyl; pyridazinyl; pyrimidinyl; pyrazinyl; indolyl; indolizinyl; isoindolyl; benzofuryl; isobenzofuryl; benzothienyl; indazolyl; benzimidazolyl; benzthiazolyl; quinolizinyl; quinolinyl; isoquinolinyl; phthalazinyl; quinazolinyl; quinoxalinyl; chromenyl; naphthyridinyl and naphthalenyl;
each R⁹ is, independently from each other, selected from the group of hydrogen; halo; cyano; hydroxy; carboxyl; nitro; amino; mono- or di(alkyl)amino; alkylcarbonylamino; aminosulfonyl; mono- or di(alkyl)aminosulfonyl; alkyl; alkyloxy; alkylcarbonyl and alkyloxycarbonyl;
X represents CR⁶R⁷, O, S, S(═O), S(═O)₂ or NR⁸; wherein:
R⁶ and R⁷ each independently are selected from the group of hydrogen, hydroxy, alkyl and alkyloxy; or
R⁶ and R⁷ taken together may form a radical selected from the group of methylene (═CH₂); mono- or di(cyano)methylene; a bivalent radical of Formula —(CH₂)₂—, —(CH₂)₃—, —(CH₂)₄—, —(CH₂)₅—, —O(CH₂)₂O—, —O(CH₂)₃—; or together with the carbon atom to which they are attached, a carbonyl;
R⁸ is selected from the group of hydrogen; alkyl; alkylcarbonyl; arylcarbonyl; arylalkyl; arylalkylcarbonyl; alkylsulfonyl; arylsulfonyl and arylalkylsulfonyl;
C is a group of formula (c-1), (c-2), (c-3), (c-4) or (c-5); 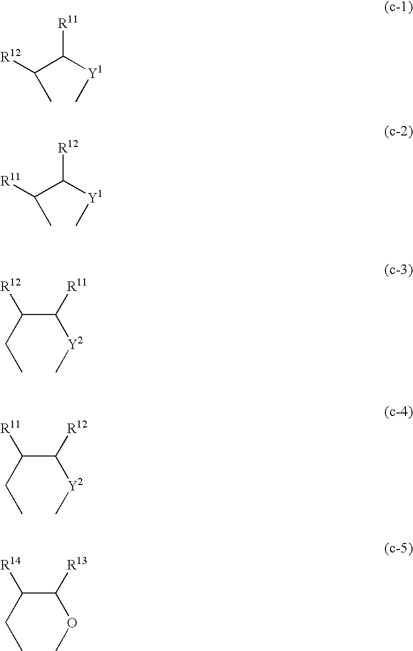
wherein
Y¹ and Y² each independently are S; O; S(═O); S(═O)2 or NR¹⁰; wherein R¹⁰ is selected from the group of hydrogen, cyano, alkyl, alkyloxyalkyl, formyl, alkylcarbonyl, alkyloxycarbonyl, alkyloxyalkylcarbonyl, arylcarbonyl, arylalkyl, arylalkylcarbonyl, alkylsulfonyl, arylsulfonyl and arylalkylsulfonyl;
R¹⁰ and R¹¹ may form together a bivalent radical (e-1) to (e-5);
—CH₂—NH—CH₂— (e-1)
—CH₂—NH—CH₂—CH₂— (e-2)
—CH₂CH₂—NH—CH₂— (e-3)
—CH=N—CH₂— (e-4)
—CH₂—N═CH₂— (e-5)
wherein optionally in each radical (e-1) to (e-5) one or more
hydrogens are replaced by one or more substituents selected from alkyl, —O-alkyl, —S-alkyl ═O, ═S, ═S(═O) and ═S(═O)₂;
R¹² is hydrogen or alkyl
R¹³, R¹⁴ each independently are hydrogen, hydroxy or oxo;
R¹¹ is a group of formula (d-1); 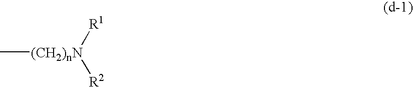
wherein:
n is zero, 1, 2, 3, 4, 5 or 6;
R¹ and R² each independently are hydrogen; alkyl; alkylcarbonyl; alkyloxyalkyl; alkylcarbonyloxyalkyl; alkyloxycarbonylalkyl; arylalkyl; arylcarbonyl; alkyloxycarbonyl; aryloxycarbonyl; arylalkylcarbonyl; alkyloxycarbonylalkylcarbonyl; mono- or di(alkyl)aminocarbonyl; mono- or di(aryl)aminocarbonyl; mono- or di(arylalkyl)aminocarbonyl; mono- or di(alkyloxycarbonylalkyl)aminocarbonyl; alkylsulphonyl; arylsulphonyl; arylalkylsulphonyl; mono- or di(alkyl)aminothiocarbonyl; mono-or di(aryl)aminothiocarbonyl; mono- or di(arylalkyl)aminothiocarbonyl; mono-, di- or tri(alkyl)amidino; mono-, di- or tri(aryl)amidino and mono-, di- or tri(arylalkyl)amidino; or
R¹ and R² taken together with the nitrogen atom to which they are attached may form a radical of formula (a-1), (a-2), (a-3), (a-4), (a-5) or (a-6); 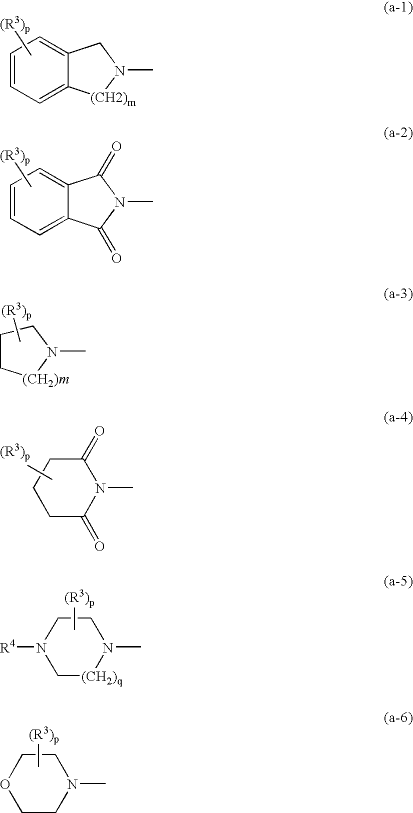
wherein:
p is zero, 1, 2, 3 or 4;
q is 1 or 2;
m is zero, 1, 2, or 3;
each R³ independently is selected from the group of hydrogen; halo; hydroxy; cyano; alkyl; alkyloxyalkyl; aryloxyalkyl; mono- or di(alkyl)aminoalkyl; hydroxycarbonylalkyl; alkyloxycarbonylalkyl; mono- or di(alkyl)aminocarbonylalkyl; mono- or di(aryl)aminocarbonylalkyl; mono- or di(alkyl)aminocarbonyloxyalkyl; alkyloxycarbonyloxyalkyl; arylaminocarbonyloxyalkyl; arylalkylaminocarbonyloxyalkyl; aryl; alkyloxy; aryloxy; alkylcarbonyloxy; arylcarbonyloxy; arylalkylcarbonyloxy; alkylcarbonyl; arylcarbonyl; aryloxycarbonyl; hydroxycarbonyl; alkyloxycarbonyl; alkylcarbonylamino; arylalkylcarbonylamino; arylcarbonylamino; alkyloxycarbonylamino; aminocarbonylamino; mono- or di(arylalkyl)aminocarbonylamino; alkyl-sulphonylalkylaminocarbonylamino; or two R³-radicals may form together a bivalent radical
—CR⁵R⁵—CR⁵R⁵—O— (b-1)
—O—CR⁵R⁵—CR⁵R⁵— (b-2)
—O—CR⁵R⁵—CR⁵R⁵— (b-3)
—O—CR⁵R⁵—CR⁵R⁵—CR⁵R⁵— (b-4)
—CR⁵R⁵—CR⁵R⁵—CR⁵R⁵— (b-5)
—O—CR⁵R⁵—CR⁵R⁵ CR⁵R⁵—O— (b-6)
—O—CR⁵R⁵—CR⁵R⁵—CR⁵R⁵—CR⁵R⁵— (b-7)
—CR⁵R⁵—CR⁵R⁵—CR⁵R⁵—CR⁵R⁵—O— (b-8)
—O—CR⁵R⁵—CR⁵R⁵CR⁵R⁵R—O— (b-9)
wherein R⁵ is selected from the group of hydrogen, halo, hydroxy, alkyloxy and alkyl;
R⁴ is selected from the group of hydrogen; alkyl; arylalkyl; alkyloxyalkyl; alkylcarbonyloxyalkyl; alkyloxycarbonylalkyl; arylcarbonylalkyl; alkylsulphonyloxyalkyl; aryloxyaryl; alkyloxycarbonylaryl; alkylcarbonyl; arylalkylcarbonyl; alkyloxycarbonylalkylcarbonyl; arylcarbonyl; alkyloxycarbonyl; aryloxycarbonyl; arylalkyloxycarbonyl; mono- or di(alkyl)aminocarbonyl; mono- or di(aryl)aminocarbonyl; mono-or di(arylalkyl)aminocarbonyl; mono- or di(alkyloxycarbonylalkyl)aminocarbonyl; alkyloxyalkylaminocarbonyl; mono-, di- or tri(alkyl)amidino; mono-, di- or tri(aryl)amidino; mono-, di- or tri(arylalkyl)amidino; alkylsulphonyl; arylalkylsulphonyl or arylsulphonyl;
aryl is phenyl or naphthyl; each radical optionally substituted with 1, 2 or 3 substituents selected from the group of halo, nitro, cyano, hydroxy, alkyloxy or alkyl;
alkyl represents a straight or branched saturated hydrocarbon radical having from 1 to 10 carbon atoms, a cyclic saturated hydrocarbon radical having from 3 to 8 carbon atoms or a saturated hydrocarbon radical containing a straight or branched moiety having from 1 to 10 carbon atoms and a cyclic moiety having from 3 to 8 carbon atoms, optionally substituted with one or more halo, cyano, oxo, hydroxy, formyl, carboxyl or amino radicals; and
halo represents fluoro, chloro, bromo and iodo.

2. A compound according to claim 1, wherein A and B are each phenyl, optionally substituted with fluor.

3. A compound according to claim 1, wherein X is CH₂ or O.

4. A compound according to claim 1, wherein
C is a group of formula (c-1) or (c-2); wherein
Y¹ is S; S(═O); S(═O)₂ or NR¹⁰; wherein R¹⁰ is selected from the group of hydrogen, cyano, alkyl, alkyloxyalkyl, formyl, alkylcarbonyl, alkyloxycarbonyl and alkyloxyalkylcarbonyl;or adjacent R¹⁰ and R¹ may form together a bivalent radical (e-1), (e-2) or (e-5); wherein optionally in each radicalone or more hydrogens are replaced by one or more substituents selected from ═O, ═S, ═S(═O), alkyl and alkylthio; and
R¹² is hydrogen.

5. A compound according to claim 1, wherein
C is a group of formula (c-3) or (c-4); wherein
Y² is O or NH; and
R¹² is hydrogen.

6. A compound according to claim 1, wherein
C is a group of formula (c-5); wherein
R¹³ is hydrogen; and
R¹⁴ is hydroxy or oxo.

7. A compound according to claim 1, wherein
R¹¹ is defined as a group of formula (d-1) wherein:
n is zero or 1;
R¹ and R² each independently are hydrogen; alkyl or alkyloxycarbonylalkyl; or R¹ and R² taken together with the nitrogen atom to which they are attached may form a radical of formula (a-3), (a-5) or (a-6); wherein
p is zero or 1;
q is 1;
m is 1;
each R³ independently is selected from the group of hydrogen and hydroxy; and
R⁴ is alkyl.

8. A compound according to claim 1, wherein:
i and j are integers, independently from each other, equal to zero or 1;
A and B are, each independently from each other, phenyl, optionally substituted with fluor;
each R⁹ is, independently from each other, selected from the group of hydrogen and halo;
X is CH₂ and O;
C is a group of formula (c-1) or (c-2); wherein
Y² is S; S(═O); S(═O)₂ or NR¹⁰; wherein R¹⁰ is selected from the group of hydrogen, cyano, alkyl, alkyloxyalkyl, formyl, alkylcarbonyl, alkyloxycarbonyl and alkyloxyalkylcarbonyl; or
adjacent R¹⁰ and R¹¹ may form together a bivalent radical (e-1), (e-2) or (e-5); wherein optionally in each radicalone or more hydrogens are replaced by one or more substituents selected from ═O, ═S, ═S(═O), alkyl and alkylthio; and
R¹² is hydrogen; or
C is a group of formula (c-3) or (c-4); wherein
Y² is O or NH; and
R¹² is hydrogen; or
C is a group of formula (c-5); wherein
R¹³ is hydrogen; and
R¹⁴ is hydroxy or oxo;
R¹¹ is a group of formula (d-1); wherein:
n is zero or 1;
R¹ and R² each independently are hydrogen; alkyl or alkyloxycarbonylalkyl; or R¹ and R² taken together with the nitrogen atom to which they are attached may form a radical of formula (a-3), (a-5) or (a-6); wherein:
p is zero or 1;
q is 1
m is 1;
each R³ independently is selected from the group of hydrogen and hydroxy; and
R⁴ is alkyl.

9. (canceled)

10. A method for the treatment of conditions, either prophylactic or therapeutic or both, mediated through the 5-HT₂, and D2 receptor, as well as the through norepinephrine reuptake inhibition, comprising administering to a warm blooded animal in need thereof a therapeutically effective amount of a compound according to claim 1.

11. A method for the treatment and/or prevention of a disorder selected from the group consisting of anxiety, depression and mild depression, bipolar disorders, sleep- and sexual disorders, psychosis, borderline psychosis, schizophrenia, migraine, personality disorders or obsessive-compulsive disorders, social phobias or panic attacks, organic mental disorders, mental disorders in children, aggression, memory disorders and attitude disorders in older people, addiction, obesity, and bulimia comprising administering to a warm blooded animal in need thereof a therapeutically effective amount of a compound according to claim 1.

12. The method according to claim 11 wherein the disorder is selected from the group consisting of anxiety, depression, psychosis, schizophrenia, migraine and addictive properties of drugs of abuse.

13. A pharmaceutical composition comprising a pharmaceutically acceptable carrier and, as active ingredient, a therapeutically effective amount of a compound according to claim 1.

14. A process for the preparation of a pharmaceutical composition comprising mixing a pharmaceutically acceptable carrier with a therapeutically effective amount of a compound as claimed in claim 1.

15. A method for the treatment or the prevention of a disorder selected from: (a) damage to the nervous system caused by trauma, stroke, neurodegenerative illnesses and the like; (b) cardiovascular disorders like high blood pressure, thrombosis, stroke, and the like; or (c) gastrointestinal disorders like dysfunction of the motility of the gastrointestinal system, comprising administering to a warm blooded animal in need thereof a therapeutically effective amount of a compound according to claim 1.
